(19) United States
(12) Patent Application Publication
(10) Pub. No.: US 2009/0053192 A1
(43) Pub. Date: Feb. 26, 2009

(54) TISSUE-NONSPECIFIC ALKALINE PHOSPHATASE (TNAP) ACTIVATORS AND USES THEREOF

(75) Inventors: Jose Luis Millan, San Diego, CA (US); Eduard Sergienko, La Jolla, CA (US)

Correspondence Address: CLARK G. SULLIVAN;ARNALL GOLDEN GREGORY LLP, 171 17TH STREET NW, SUITE 2100, ATLANTA, GA, 30363, US

(73) Assignee: BURNHAM INSTITUTE FOR MEDICAL RESEARCH, La Jolla, CA (US)

(21) Appl. No.: 12/189,149

(22) Filed: Aug. 9, 2008

Related U.S. Application Data
(60) Provisional application No. 60/955,289, filed on Aug. 10, 2007.
(60) Provisional application No. 61/038,456, filed on Mar. 21, 2008.

(51) Int. Cl. A61K 38/46; C12N 9/14; A61K 31/519; A61K 31/53; A61K 31/5377; A61K 31/5395; A61P 19/10

(52) U.S. Cl. 424/94.6; 435/195; 514/265.1; 514/245; 514/235.2; 514/229.2

(57) ABSTRACT

Disclosed herein are tissue-nonspecific alkaline phosphatase (TNAP) activators and uses thereof for promoting bone mineral deposition.

### CROSS-REFERENCE TO RELATED APPLICATIONS

This application claims benefit of U.S. Provisional Application No. 60/955,289, filed Aug. 10, 2007, and of U.S. Provisional Application No. 61/038,456, filed Mar. 21, 2008. Application No. 60/955,289, filed Aug. 10, 2007, and of Application No. 61/038,456, are hereby incorporated herein by reference in their entirety.

### STATEMENT REGARDING FEDERALLY SPONSORED RESEARCH

This invention was made with government support under Grant RO1 DE012889 and RO3 MH082385 awarded by the National Institutes of Health and Grant DE12889 awarded by the Department of Energy. The government has certain rights in the invention.

### BACKGROUND

During the process of endochondral bone formation, osteoblasts mineralize the extracellular matrix (ECM) by promoting the initial formation of crystalline hydroxyapatite in the sheltered interior of membrane-limited matrix vesicles (MVs) and by modulating matrix composition to further promote propagation of apatite outside of the MVs. Controlled bone mineralization depends on a regulated balance of the following factors: the concentrations of Ca²⁺ and inorganic phosphate (Pᵢ), the presence of fibrilar collagens (e.g., type I in bone; Types II and X in cartilage) and the presence of adequate concentrations of mineralization inhibitors, i.e., inorganic pyrophosphate (PPᵢ), and osteopontin.

Tissue-nonspecific alkaline phosphatase (TNAP) is the only tissue-nonrestricted isozyme of a family of four homologous human AP genes (EC. 3.1.3.1) and is expressed as an ectoenzyme anchored via a phosphatidylinositol glycan moiety. A deficiency in the TNAP isozyme causes the inborn-error-of-metabolism known as hypophosphatasia, which is important for bone mineralization (Whyte, 1994). The severity of hypophosphatasia is variable and modulated by the nature of the TNAP mutation (Henthorn et al., 1992; Fukushi et al., 1998; Shibata et al., 1998; Zurutuza et al., 1999). Unlike most types of rickets or osteomalacia neither calcium nor inorganic phosphate levels in serum are subnormal in hypophosphatasia. In fact hypercalcemia and hyperphosphatemia may exist and hypercalciuria is common in infantile hypophosphatasia (Whyte, 1995). The clinical severity in hypophosphatasia patients varies widely. The different syndromes, listed from the most severe to the mildest forms, are: perinatal hypophosphatasia, infantile hypophosphatasia, childhood hypophosphatasia, adult hypophosphatasia, odontohypophosphatasia and pseudohypophosphatasia (Whyte, 1995). These phenotypes range from complete absence of bone mineralization and stillbirth to spontaneous fractures and loss of decidual teeth in adult life. Inactivation of the mouse TNAP gene (Akp2), phenocopies the infantile form of human hypophosphatasia (Narisawa et al., 1997; Fedde et al., 1999). In bone, TNAP is confined to the cell surface of osteoblasts and chondrocytes, including the membranes of their shed MVs (Ali et al., 1970; Bernard, 1978). In fact, MVs are markedly enriched in TNAP compared to both whole cells and the plasma membrane (Morris et al., 1992). There is no established medical therapy for hypophosphatasia. Case reports of enzyme replacement therapy (ERT) using intravenous (i.v.) infusions of ALP-rich plasma from Paget's bone disease patients, purified human liver ALP or purified placental ALP have described failure to rescue affected infants. ALP activity must be increased not in the circulation, but in the skeleton itself. Disclosed herein are compositions and methods for treating or enhancing treatment of hypophosphatasia.

Osteoporosis, or porous bone, is a disease characterized by low bone mass and structural deterioration of bone tissue, leading to bone fragility and an increased susceptibility to fractures of the hip, spine, and wrist. Men as well as women suffer from osteoporosis. According to statistics published by the Osteoporosis and Related Bone Diseases National Resource Center of the National Institutes of health, USA, osteoporosis is a major public health threat for 28 million Americans, 80% of whom are women. One out of every two women and one in eight men over 50 will have an osteoporosis-related fracture in their lifetime. Estimated national direct expenditures (hospitals and nursing homes) for osteoporosis and related fractures are $14 billion each year. Osteoporosis results from an imbalance between osteoblast-mediated bone formation and osteoclast-mediated bone resorption with a net result favoring bone resorption (Rodan et al., 2002). Bone continuously remodels in response to mechanical and physiological stresses, and this remodeling allows vertebrates to renew bone as adults. Bone remodeling consists of the cycled resorption and synthesis of collagenous and noncollagenous extracellular matrix proteins. Bone resorption is performed by osteoclasts whereas synthesis is performed by osteoblasts, and an imbalance in this process can lead to disease states such as osteoporosis, or more rarely, osteopetrosis. In many postmenopausal women, the extent of bone resorption exceeds that of formation, resulting in osteoporosis and increased fracture risk. Approximately 100 million people suffer from postmenopausal osteoporosis worldwide. Hormone replacement therapy, selective estrogen receptor modulators, calcitonin, and bisphosphonates are useful for prevention and or treatment of postmenopausal osteoporosis (Sherman, 2001). In general, treatments for osteoporosis are aimed are reducing bone resorption by decreasing osteoclastic activity via administration of bisphosphonates (Fleisch et al., 2002) which induce osteoclast apoptosis, or by stimulating osteoblastic activity using peptides that mimic some of the functions of parathyroid hormone (Hodsman et al., 2002). In addition, several therapies may increase bone formation in osteoporotic patients, such as the lipid-lowering drugs “statins” (Wang et al., 2000), fibroblast growth factor-1 (Dusntan et al., 1999), and parathyroid hormone (PTH) (Reeve, 2002). Daily supplementation of calcium and Vitamin D (which promotes absorption of calcium through the gut) are also seen as important for the maintenance of healthy bone mineral mass (Heaney, 2002), as calcium is the principal ion present in hydroxyapatite. Disclosed herein are compositions and methods for treating or enhancing treatment of osteoporosis.

### BRIEF SUMMARY

In accordance with the purpose of this invention, as embodied and broadly described herein, this invention relates to tissue-nonspecific alkaline phosphatase (TNAP) activators and uses thereof for promoting bone mineral deposition. Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows short-term, low dose (1 mg/Kg), ERT efficacy studies in Akp2^{-/-} mice. FIG. 1A shows serum sALP-FcD₁₀ concentrations at day 16 in mice treated for 15 days with daily s.c. injections of sALP-FcD₁₀. For WT mice, values represent TNALP concentrations calculated from a calibration curve of activity versus known amounts of purified TNALP protein. FIG. 1B shows serum PPᵢ concentrations. Note that this low dose of 1 mg/kg sALP-FcD₁₀ was sufficient to maintain normal PPᵢ levels. FIG. 1C shows hypertrophic zone area expressed as a percentage of the total growth plate area. Note the normal hypertrophic zone area in the ERT mice.

FIG. 2 shows short-term, medium dose (2 mg/Kg), ERT efficacy studies in Akp2^{-/-} mice. FIG. 2A shows serum levels of sALP-FcD₁₀ were detected in ˜50% of the ERT mice. FIG. 2B shows growth curves of untreated Akp2^{-/-} mice (n=7) compared to WT controls (n=7). FIG. 2C shows growth curves of sALP-FcD₁₀-treated Akp2^{-/-} mice (n=8) compared to WT mice (n=8). Note the sustained, normal growth, which occurred without epileptic seizures and with apparent well-being, in the treated mice.

FIG. 3 shows short-term, high dose (8.2 mg/Kg), ERT efficacy studies in Akp2^{-/-} mice. FIG. 3A shows plasma concentrations of ALP activity. FIG. 3B shows growth curves of Akp2^{-/-} mice given vehicle (n=18) or sALP-FcD10 (n=19) and non-treated WT mice (n=18). FIG. 3C shows effect of sALP-FcD10 on tibial (left panel) and femur (right panel) length (measurements from day 16).

FIG. 4 shows long-term (52 days), high dose (8.2 mg/Kg), ERT efficacy studies in Akp2^{-/-} mice. FIG. 6A shows long-term survival of ERT mice contrasts with precipitous, early demise of the vehicle treated group. FIG. 6B shows plasma ALP levels in untreated and treated Akp2^{-/-} mice and WT controls.

FIG. 5 shows Micro CT analysis. FIG. 5A shows BMD (bone mineral density). Spine trabecular bone of transgenic mice showed higher BMD than wild-type mice, while calvaria bone and the cortical and distal regions of femur did not show difference. FIG. 5B shows BVF (bone volume fraction). Significant increase of mineralization was observed in trabecular bones of femur and spine in the transgenic animals. All genotypes: n=6, 3 female and 3 male adulte mice.

FIG. 6 shows a luminescence-based assay for TNAP. FIG. 6A shows reaction mechanism—dioxetane-phosphate is dephosphorylated by an alkaline phosphatase leading to generation of an unstable product that decomposes with concomitant light production. FIG. 6B shows spectrum of light emitted in the CDP-star dephosphorylation reaction.

FIG. 7 shows optimization of TNAP concentration for the detection of activation with a luminescent readout. The activity of TNAP (serial dilutions) was measured in 50 mM CAPS, pH 9.8, containing 1 mM MgCl2, 20 uM ZnCl2 and 50 uM CDP-Star®. The TNAP concentration is expressed in fold over 1/800 dilution, e.g. the highest concentration of TNAP in this experiment was equal 1/100. The luminescence signal was obtained using 384-well white small-volume plates (Greiner 784075) on a PE Envision plate reader.

FIG. 8 shows optimization of CDP-star® concentration for the TNAP activation assay. The activity of TNAP (1/800) was measured in 50 mM CAPS, pH 9.8, containing 1 mM MgCl2, 20 uM ZnCl2 and varied concentrations of CDP-Star. The luminescence signal was obtained using 384-well white small-volume plates (Greiner 784075) on a PE Envision plate reader. Experimental data were analyzed using the Michaelis-Menten equation. The following kinetic parameters were obtained: Vₘₐₓ=10300±408 (RLU) and Kₘ(CDP-star)=21.9±3.4 μM.

FIG. 9 shows effect of diethanolamine concentration on the TNAP reaction rate. The activity of TNAP (1/800) was measured in 50 mM CAPS, pH 9.8, containing 1 mM MgCl₂, 20 μM ZnCl2 and 25 μM CDP-Star® in the presence of serially diluted DEA, pH adjusted to 9.8. The luminescence signal, obtained using 384-well white small-volume plate (Greiner 784075) on a PE Envision plate reader, was fitted to 4-parameter sigmoidal equation. The best-fit curve is shown as a solid line.

FIG. 10 shows performance of the TNAP activation assay. The activity of TNAP (1/800) was measured in 50 mM CAPS, pH 9.8, containing 1 mM MgCl₂, 20 μM ZnCl₂ and 25 ∥M CDP-Star® in the presence and absence of 600 mM DEA, adjusted to pH 9.8. The luminescence signal was obtained using 384-well white small-volume plates (Greiner 784075) on a PE Envision plate reader. All reagents were dispensed using Matrix WellMate bulk reagent dispenser.

FIG. 11 shows purification and properties of recombinant sALP-FcD₁₀, and pharmacokinetic and tissue, distribution studies. FIG. 11A shows SDS-PAGE of purified sALP-FcD₁₀. Protein purified by affinity chromatography Protein A-Sepharose was analyzed by SDS-PAGE and bands stained with Sypro Ruby. sALP-FcD₁₀ migrated as the major species with an apparent molecular mass of ˜90,000 Da under reducing conditions (Red), and ˜200,000 Da under non-reducing, native conditions (Nat). FIG. 11B shows characterization of sALP-FcD₁₀ by molecular sieve chromatography under non-denaturing conditions. Purified sALP-FcD₁₀ protein (2 mg) was resolved on a calibrated column of Sephacryl S-300. The principal form of sALP-FcD₁₀ (Peak 3), consisting of 80% of the total material deposited on the column, eluted with a molecular mass of 370,000 Da consistent with a tetrameric structure. When analyzed by SDS-PAGE in the presence of dithiothreitol (DTT), the material in peak 3 migrated with an apparent molecular mass of a monomer. In the absence of DTT, the protein migrated with the mobility of a dimer. FIG. 11C shows concentrations of radiolabeled sALP-FcD¹⁰ in serum, tibia, and muscle, expressed as μg/g tissue (wet weight), after a single i.v. bolus of 5 mg/kg in adult WT mice (n=3). FIG. 11D shows serum concentrations of radiolabeled sALP-FcD₁₀ as a function of time after a single s.c. injection of 3.7 mg/kg in 1 day-old WT mice (n=3).

### DETAILED DESCRIPTION

The disclosed method and compositions may be understood more readily by reference to the following detailed description of particular embodiments and the Example included therein and to the Figures and their previous and following description.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a compound is disclosed and discussed and a number of modifications that can be made to a number of molecules including the compound are discussed, each and every combination and permutation of compound and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C—F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Those skilled in the art will recognize, or be able to ascertain using no more than routine experimentation, many equivalents to the specific embodiments of the method and compositions described herein. Such equivalents are intended to be encompassed by the following claims.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

### A. Compositions

This application is related to the subject matter of U.S. patent application Ser. No. 11/576,251, filed Mar. 28, 2007, the contents of which are incorporated herein by reference.

As disclosed herein, Akp2−/− mice treated with recombinant human TNAP optimized for delivery to bone preserved the survival and well-being of Akp2^{-/-} mice, preventing epileptic seizures and the severe skeletal and dental abnormalities characteristic of this excellent mouse model for Infantile hypophosphatasia. These findings represent the first successful use of ERT for a heritable primary disease of the skeleton, and are a foundation for therapeutic trials for human hypophosphatasia. In addition, the simultaneous administration of a TNAP activator can facilitate ERT, by allowing a significant reduction in the amount of enzyme required for a comparable effect. Furthermore, since most patients with hypophosphatasia do not harbor null mutations in the TNAP gene, but rather different missense mutations with various residual activities of the enzyme, the administration of TNAP activators by themselves represents a useful therapeutic strategy for hypophosphatasia by activating the residual activity in these patients.

Disclosed herein is a method of promoting bone mineral deposition and treating hypophosphatasia and osteoporosis via manipulating the Pᵢ/PPᵢ ratio. As disclosed herein, one way of manipulating this ratio is to increase the degradation of PPᵢ by activating TNAP's pyrophosphatase activity.

1. TNAP Activators

Disclosed herein are tissue-nonspecific alkaline phosphatase (TNAP) activators that can be used, for example, in treating or preventing conditions relating to dysregulated calcification. The disclosed composition can be used, for example, for the treatment of heritable bone disorders. The composition can further comprise a pharmaceutically acceptable carrier.

The first category of tissue-nonspecific alkaline phosphatase activators of the present disclosure are amides having the formula:

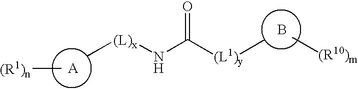

wherein A represents a 5-member heterocyclic or heteroaryl ring that can optionally have from 1 to 4 hydrogen atoms substituted by an organic radical, R¹, wherein the index n represents the number of R¹ units that are present and the index n has a value from 1 to 4. B represents a phenyl, cyclopentyl, cyclohexyl, or a 5-member heterocyclic ring wherein and R¹⁰ represents from 1 to 5 organic radicals that can optionally substitute for a hydrogen atom. Each R¹ and R¹⁰ unit is independently selected.

A units can comprise 5-member heteroaryl rings. The following are non-limiting examples of 5-member heteroaryl and heterocyclic rings:

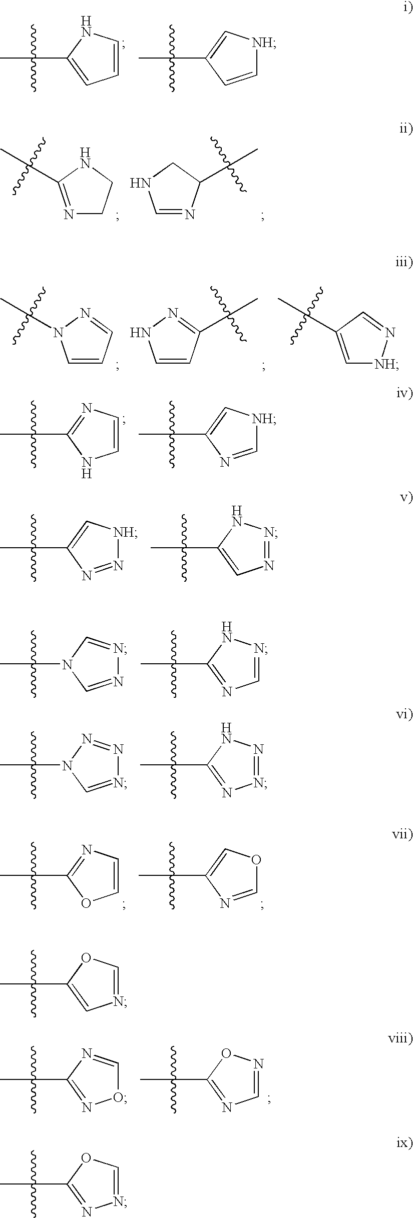

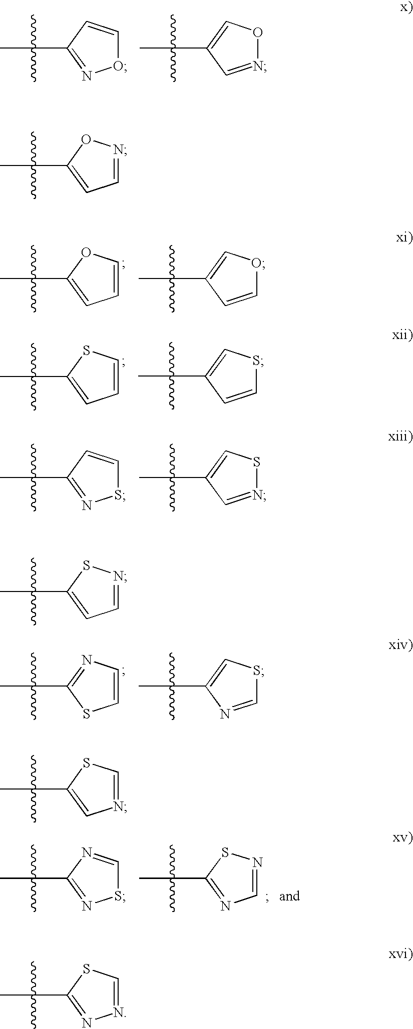

One embodiment of A rings relates to 5-member heteroaryl rings chosen from:

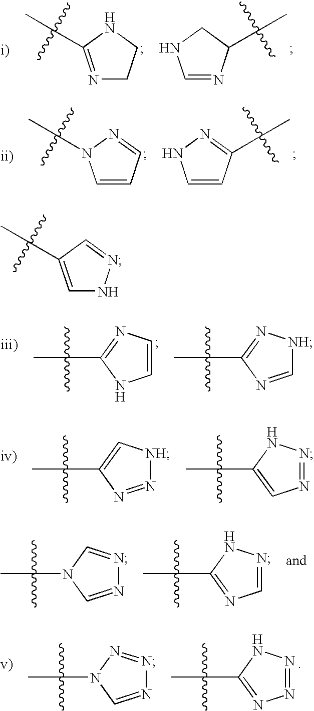

Another embodiment encompasses 1,2,4-triazoles having the formula:

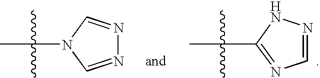

A further embodiment encompasses thiazoles having the formula:

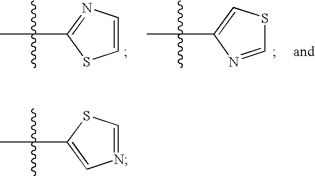

The 5-member heteroaryl rings can have from one to four R¹ organic radicals that substitute for hydrogen atoms on the rings, for example,

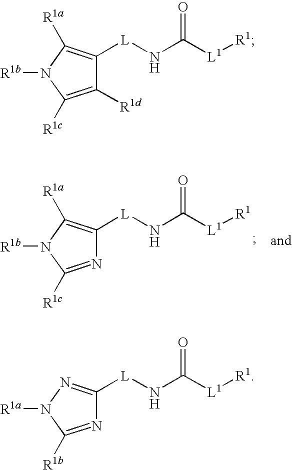

The individual R¹ organic radicals, R^{1a}, R^{1b}, R^{1c}, and R^{1d}, are each independently chosen from one another. The following are non-limiting examples of organic radicals that can substitute for a hydrogen of an A ring:
i) linear, branched, or cyclic alkyl, alkenyl, and alkynyl; for example, methyl (C₁), ethyl (C₂), n-propyl (C₃), iso-propyl (C₃), cyclopropyl (C₃), propylen-2-yl (C₃), propargyl (C₃), n-butyl (C₄), iso-butyl (C₄), sec-butyl (C₄), tert-butyl (C₄), cyclobutyl (C₄), n-pentyl (C₅), cyclopentyl (C₅), n-hexyl (C₆), and cyclohexyl (C₆);
ii) substituted or unsubstituted aryl; for example, phenyl, 2-fluorophenyl, 3-chlorophenyl, 4-methylphenyl, 2-aminophenyl, 3-hydroxyphenyl, 4-trifluoromethylphenyl, and biphenyl-4-yl;
iii) substituted or unsubstituted heterocyclic; for example, piperidinyl, pyrrolidinyl, and morpholinyl;
iv) substituted or unsubstituted heteroaryl; for example, pyrrolyl, pyridinyl, and pyrimidinyl;
v) —(CR^{3a}R^{3b})_{q}OR²; for example, —OH, —CH₂OH, —OCH₃, —CH₂OCH₃, —OCH₂CH₃, —CH₂OCH₂CH₃, —OCH₂CH₂CH₃, and —CH₂OCH₂CH₂CH₃;
vi) —(CR^{3a}R^{3b})_{q}C(O)R²; for example, —COCH₃, —CH₂COCH₃, —OCH₂CH₃, —CH₂COCH₂CH₃, —COCH₂CH₂CH₃, and —CH₂COCH₂CH₂CH₃;
vii) —(CR^{1a}R^{3b})_{q}C(O)OR²; for example, —CO₂CH₃, —CH₂CO₂CH₃, —CO₂CH₂CH₃, —CH₂CO₂CH₂CH₃, —CO₂CH₂CH₂CH₃, and —CH₂CO₂CH₂CH₂CH₃;
viii) —(CR^{3a}R^{3b})_{q}C(O)N(R²)₂; for example, —CONH₂, —CH₂CONH₂, —CONHCH₃, —CH₂CONHCH₃, —CON(CH₃)₂, and —CH₂CON(CH₃)₂;
ix) —(CR^{3a}R^{3b})_{q}OC(O)N(R²)₂; for example, —OC(O)NH₂, —CH₂OC(O)NH₂, —OC(O)NHCH₃, —CH₂OC(O)NHCH₃, —OC(O)N(CH₃)₂, and —CH₂OC(O)N(CH₃)₂;
x) —(CR^{3a}R^{3b})_{q}N(R²)₂; for example, —NH₂, —CH₂NH₂, —NHCH₃, —N(CH₃)₂, —NH(CH₂CH₃), —CH₂NHCH₃, —CH₂N(CH₃)₂, and —CH₂NH(CH₂CH₃);
xi) halogen: —F, —Cl, —Br, and —I;
xii) —CHₘXₙ; wherein X is halogen, m is from 0 to 2, m+n=3; for example, —CH₂F, —CHF₂, —CF₃, —CCl₃, or —CBr₃;
xiii) —(CR^{3a}R^{3b})_{q}CN; for example; —CN, —CH₂CN, and —CH₂CH₂CN;
xiv) —(CR^{3a}R^{3b})_{q}NO₂; for example; —NO₂, —CH₂NO₂, and —CH₂CH₂NO₂;
xv) —(CR^{3a}R^{3b})_{q}SO₃R²; for example, —SO₂H, —CH₂SO₂H, —SO₂CH₃, —CH₂SO₂CH₃, —SO₂C₆H₅, and —CH₂SO₂C₆H₅; and
xvi) —(CR^{3a}R^{3b})_{q}SO₃R²; for example, —SO₃H, —CH₂SO₃H, —SO₃CH₃, —CH₂SO₃CH₃, —SO₃C₆H₅, and —CH₂SO₃C₆H₅;
   wherein each R² is independently hydrogen, substituted or unsubstituted C₁-C₄ linear, branched, or cyclic alkyl; or two R² units can be taken together to form a ring comprising 3-7 atoms; R^{3a} and R^{3b} are each independently hydrogen or C₁-C₄ linear or branched alkyl; the index q is from 0 to 4.

One embodiment of R¹ organic radicals as substitutions for hydrogen includes aryl substituted 1,2,4-triazoles, for example:

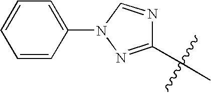

When R¹ comprises C₁-C₁₂ linear, branched, or cyclic alkyl, alkenyl; substituted or unsubstituted C₆ or C₁₀aryl; substituted or unsubstituted C₁-C₉heterocyclic; or substituted or unsubstituted C₁-C₉heteroaryl; R¹ can further have one or more hydrogen atoms substituted by one or more organic radicals. Non-limiting examples of organic radicals that can substitute for a hydrogen atom of R¹ include:
i) linear, branched, or cyclic alkyl, alkenyl, and alkynyl; for example, methyl (C₁), ethyl (C₂), n-propyl (C₃), iso-propyl (C₃), cyclopropyl (C₃), propylen-2-yl (C₃), propargyl (C₃), n-butyl (C₄), iso-butyl (C₄), sec-butyl (C₄), tert-butyl (C₄), cyclobutyl (C₄), n-pentyl (C₅), cyclopentyl (C₅), n-hexyl (C₆), and cyclohexyl (C₆);
ii) —(CR^{5a}R^{5b})_{q}OR⁴; for example, —OH, —CH₂OH, —OCH₃, —CH₂OCH₃, —OCH₂CH₃, —CH₂OCH₂CH₃, —OCH₂CH₂CH₃, and —CH₂OCH₂CH₂CH₃;
iii) —(CR^{5a}R^{5b})_{q}C(O)R⁴; for example, —COCH₃, —CH₂COCH₃, —OCH₂CH₃, —CH₂COCH₂CH₃, —COCH₂CH₂CH₃, and —CH₂COCH₂CH₂CH₃;
iv) —(CR^{5a}R^{5b})_{q}C(O)OR⁴; for example, —CO₂CH₃, —CH₂CO₂CH₃, —CO₂CH₂CH₃, —CH₂CO₂CH₂CH₃, —CO₂CH₂CH₂CH₃, and —CH₂CO₂CH₂CH₂CH₃;
v) —(CR^{5a}R^{5b})_{q}C(O)N(R⁴)₂; for example, —CONH₂, —CH₂CONH₂, —CONHCH₃, —CH₂CONHCH₃, —CON(CH₃)₂, and —CH₂CON(CH₃)₂;
vi) —(CR^{5a}R^{5b})_{q}OC(O)N(R⁴)₂; for example, —OC(O)NH₂, —CH₂OC(O)NH₂, —OC(O)NHCH₃, —CH₂OC(O)NHCH₃, —OC(O)N(CH₃)₂, and —CH₂OC(O)N(CH₃)₂;
vii) —(CR^{5a}R^{5b})_{q}N(R⁴)₂; for example, —NH₂, —CH₂NH₂, —NHCH₃, —N(CH₃)₂, —NH(CH₂CH₃), —CH₂NHCH₃, —CH₂N(CH₃)₂, and —CH₂NH(CH₂CH₃);
viii) halogen: —F, —Cl, —Br, and —I;
ix) —CHₘXₙ; wherein X is halogen, m is from 0 to 2, m+n=3; for example, —CH₂F, —CHF₂, —CF₃, —CCl₃, or —CBr₃;
x) —(CR^{5a}R^{5b})_{q}CN; for example; —CN, —CH₂CN, and —CH₂CH₂CN;
xi) —(CR^{5a}R^{5b})_{q}NO₂; for example; —NO₂, —CH₂NO₂, and —CH₂CH₂NO₂;
xii) —(CR^{5a}R^{5b})_{q}SO₂R⁴; for example, —SO₂H, —CH₂SO₂H, —SO₂CH₃, —CH₂SO₂CH₃, —SO₂C₆H₅, and —CH₂SO₂C₆H₅; and
xiii) —(CR^{5a}R^{5b})_{q}SO₃R⁴; for example, —SO₃H, —CH₂SO₃H, —SO₃CH₃, —CH₂SO₃CH₃, —SO₃C₆H₅, and —CH₂SO₃C₆H₅;
   wherein each R⁴ is independently hydrogen, substituted or unsubstituted C₁-C₄ linear, branched, or cyclic alkyl; or two R⁴ units can be taken together to form a ring comprising 3-7 atoms; R^{5a} and R^{5b} are each independently hydrogen or C₁-C₄ linear or branched alkyl; the index p is from 0 to 4.

One embodiment of organic radicals that can substitute for a hydrogen atom on an R¹ organic radical includes A rings wherein R¹ is a substituted phenyl, for example:

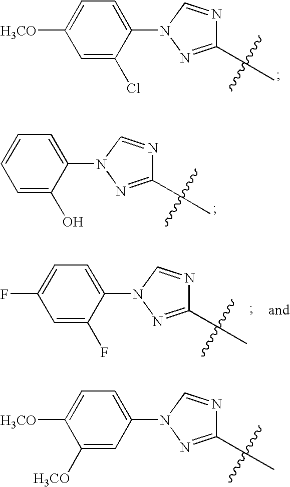

One aspect of A rings relates to 5-member heteroaryl rings that are unsubstituted. Another aspect of A rings are 5-member heteroaryl rings that are substituted with at least one organic radical R¹ that is chosen from C₁-C₄alkyl, alkenyl, or alkynyl, for example, methyl (C₁), ethyl (C₂), n-propyl (C₃), iso-propyl (C₃), cyclopropyl (C₃), propylen-2-yl (C₃), propargyl (C₃), n-butyl (C₄), iso-butyl (C₄), sec-butyl (C₄), tert-butyl (C₄), or cyclobutyl (C₄).

Another aspect of A rings relates to 5-member heteroaryl rings that are substituted with a phenyl ring or a phenyl ring further substituted with one or more organic radicals. For example, a 1,2,4-triazole ring substituted by at least one organic radical chosen from 2-fluorophenyl, 2-chlorophenyl, 2-methylphenyl, 2-methoxy-phenyl, 3-fluorophenyl, 3-chlorophenyl, 3-methylphenyl, 3-methoxyphenyl, 4-fluorophenyl, 4-chlorophenyl, 4-methylphenyl, and 4-methoxyphenyl. However, the phenyl ring can be substituted by from 1 to 5 of the organic radicals disclosed herein above.

B rings are phenyl, cyclopentyl, cyclohexyl, or a 5-member heterocyclic ring each of which can be further substituted by from 1 to 5 R¹⁰ units. The following are non-limiting examples of heterocyclic rings:

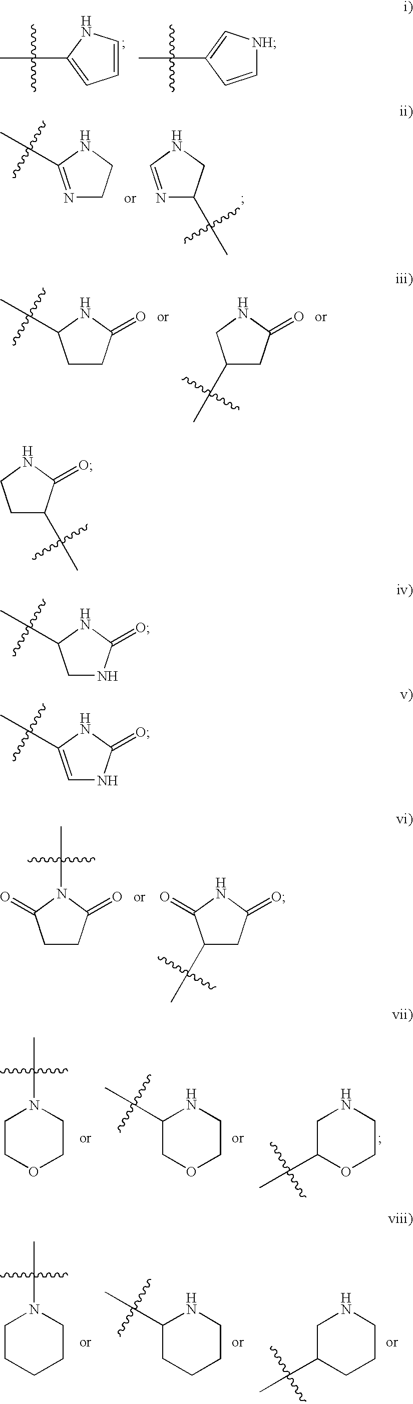

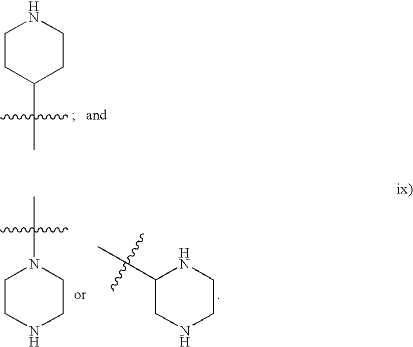

R¹⁰ represents from 1 to 5 optionally present organic radical that can substitute for a hydrogen atom on a B ring. The R¹⁰ organic radicals are independently selected. The following is a non-limiting list of R¹⁰ that can substitute for hydrogen on a B ring:
i) linear, branched, or cyclic alkyl, alkenyl, and alkynyl; for example, methyl (C₁), ethyl (C₂), n-propyl (C₃), iso-propyl (C₃), cyclopropyl (C₃), propylen-2-yl (C₃), propargyl (C₃), n-butyl (C₄), iso-butyl (C₄), sec-butyl (C₄), tert-butyl (C₄), cyclobutyl (C₄), n-pentyl (C₅), cyclopentyl (C₅), n-hexyl (C₆), and cyclohexyl (C₆);
ii) substituted or unsubstituted aryl; for example, phenyl, 2-fluorophenyl, 3-chlorophenyl, 4-methylphenyl, 2-aminophenyl, 3-hydroxyphenyl, 4-trifluoromethylphenyl, and biphenyl-4-yl;
iii) substituted or unsubstituted heterocyclic; for example, piperidinyl, pyrrolidinyl, and morpholinyl;
iv) substituted or unsubstituted heteroaryl; for example, pyrrolyl, pyridinyl, and pyrimidinyl;
v) —(CR^{12a}R^{12b})_{q}OR¹¹; for example, —OH, —CH₂OH, —OCH₃, —CH₂OCH₃, —OCH₂CH₃, —CH₂OCH₂CH₃, —OCH₂CH₂CH₃, and —CH₂OCH₂CH₂CH₃;
vi) —(CR^{12a}R^{12b})_{q}C(O)R¹¹; for example, —COCH₃, —CH₂COCH₃, —OCH₂CH₃, —CH₂COCH₂CH₃, —COCH₂CH₂CH₃, and —CH₂COCH₂CH₂CH₃;
vii) —(CR^{12a}R^{12b})_{q}C(O)OR¹¹; for example, —CO₂CH₃, —CH₂CO₂CH₃, —CO₂CH₂CH₃, —CH₂CO₂CH₂CH₃, —CO₂CH₂CH₂CH₃, and —CH₂CO₂CH₂CH₂CH₃;
viii) —(CR^{12a}R^{12b})_{q}C(O)N(R¹¹)₂; for example, —CONH₂, —CH₂CONH₂, —CONHCH₃, —CH₂CONHCH₃, —CON(CH₃)₂, and —CH₂CON(CH₃)₂;
   ix) —(CR^{12a}R^{12b})_{q}OC(O)N(R¹¹)₂; for example, —OC(O)NH₂, —CH₂OC(O)NH₂, —OC(O)NHCH₃, —CH₂OC(O)NHCH₃, —OC(O)N(CH₃)₂, and —CH₂OC(O)N(CH₃)₂;
   x) —(CR^{12a}R^{12b})_{q}N(R¹¹)₂; for example, —NH₂, —CH₂NH₂, —NHCH₃, —N(CH₃)₂, —NH(CH₂CH₃), —CH₂NHCH₃, —CH₂N(CH₃)₂, and —CH₂NH(CH₂CH₃);
   xi) halogen: —F, —Cl, —Br, and —I;
   xii) —CHₘXₙ; wherein X is halogen, m is from 0 to 2, m+n=3; for example, —CH₂F, —CHF₂, —CF₃, —CCl₃, or —CBr₃;
   xiii) —(CR^{12a}R^{12b})_{q}CN; for example; —CN, —CH₂CN, and —CH₂CH₂CN;
   xiv) —(CR^{12a}R^{12b})_{q}NO₂; for example; —NO₂, —CH₂NO₂, and —CH₂CH₂NO₂;
   xv) —(CR^{12a}R^{12b})_{q}SO₂R¹¹; for example, —SO₂H, —CH₂SO₂H, —SO₂CH₃, —CH₂SO₂CH₃, —SO₂C₆H₅, and —CH₂SO₂C₆H₅; and
   xvi) —(CR^{12a}R^{12b})_{q}SO₃R¹¹; for example, —SO₃H, —CH₂SO₃H, —SO₃CH₃, —CH₂SO₃CH₃, —SO₃C₆H₅, and —CH₂SO₃C₆H₅;
      wherein each R¹¹ is independently hydrogen, substituted or unsubstituted C₁-C₄ linear, branched, or cyclic alkyl; or two R¹¹ units can be taken together to form a ring comprising 3-7 atoms; R^{12a} and R^{12b} are each independently hydrogen or C₁-C₄ linear or branched alkyl; the index q is from 0 to 4.

When R¹⁰ comprises C₁-C₁₂ linear, branched, or cyclic alkyl, alkenyl; substituted or unsubstituted C₆ or C₁₀aryl; substituted or unsubstituted C₁-C₉heterocyclic; or substituted or unsubstituted C₁-C₉heteroaryl; the organic radical can further have one or more hydrogen atoms substituted by one or more organic radicals. Non-limiting examples of organic radicals that can substitute for a hydrogen atom of R¹⁰ include:
i) linear, branched, or cyclic alkyl, alkenyl, and alkynyl; for example, methyl (C₁), ethyl (C₂), n-propyl (C₃), iso-propyl (C₃), cyclopropyl (C₃), propylen-2-yl (C₃), propargyl (C₃), n-butyl (C₄), iso-butyl (C₄), sec-butyl (C₄), tert-butyl (C₄), cyclobutyl (C₄), n-pentyl (C₅), cyclopentyl (C₅), n-hexyl (C₆), and cyclohexyl (C₆);
ii) —(CR^{14a}R^{14b})_{q}OR¹³; for example, —OH, —CH₂OH, —OCH₃, —CH₂OCH₃, —OCH₂CH₃, —CH₂OCH₂CH₃, —OCH₂CH₂CH₃, and —CH₂OCH₂CH₂CH₃;
iii) —(CR^{14a}R^{14b})_{q}C(O)R¹³; for example, —COCH₃, —CH₂COCH₃, —OCH₂CH₃, —CH₂COCH₂CH₃, —COCH₂CH₂CH₃, and —CH₂COCH₂CH₂CH₃;
iv) —(CR^{14a}R^{14b})_{q}C(O)OR¹³; for example, —CO₂CH₃, —CH₂CO₂CH₃, —CO₂CH₂CH₃, —CH₂CO₂CH₂CH₃, —CO₂CH₂CH₂CH₃, and —CH₂CO₂CH₂CH₂CH₃;
v) —(CR^{14a}R^{14b})_{q}C(O)N(R¹³)₂; for example, —CONH₂, —CH₂CONH₂, —CONHCH₃, —CH₂CONHCH₃, —CON(CH₃)₂, and —CH₂CON(CH₃)₂;
vi) —(CR^{14a}R^{14b})_{q}OC(O)N(R¹³)₂; for example, —OC(O)NH₂, —CH₂OC(O)NH₂, —OC(O)NHCH₃, —CH₂OC(O)NHCH₃, —OC(O)N(CH₃)₂, and —CH₂OC(O)N(CH₃)₂;
vii) —(CR^{14a}R^{14b})_{q}N(R¹³)₂; for example, —NH₂, —CH₂NH₂, —NHCH₃, —N(CH₃)₂, —NH(CH₂CH₃), —CH₂NHCH₃, —CH₂N(CH₃)₂, and —CH₂NH(CH₂CH₃);
viii) halogen: —F, —Cl, —Br, and —I;
ix) —CHₘXₙ; wherein X is halogen, m is from 0 to 2, m+n=3; for example, —CH₂F, —CHF₂, —CF₃, —CCl₃, or —CBr₃;
x) —(CR^{14a}R^{14b})_{q}CN; for example; —CN, —CH₂CN, and —CH₂CH₂CN;
xi) —(CR^{14a}R^{14b})_{q}NO₂; for example; —NO₂, —CH₂NO₂, and —CH₂CH₂NO₂;
xii) —(CR^{14a}R^{14b})_{q}SO₂R¹³; for example, —SO₂H, —CH₂SO₂H, —SO₂CH₃, —CH₂SO₂CH₃, —SO₂C₆H₅, and —CH₂SO₂C₆H₅; and
xiii) —(CR^{14a}R^{14b})_{q}SO₃R¹³; for example, —SO₃H, —CH₂SO₃H, —SO₃CH₃, —CH₂SO₃CH₃, —SO₃C₆H₅, and —CH₂SO₃C₆H₅;
   wherein each R¹³ is independently hydrogen, substituted or unsubstituted C₁-C₄ linear, branched, or cyclic alkyl; or two R¹³ units can be taken together to form a ring comprising 3-7 atoms; R^{14a} and R^{14b} are each independently hydrogen or C₁-C₄ linear or branched alkyl; the index p is from 0 to 4.

One embodiment of B rings relates to B rings that are unsubstituted phenyl. Another embodiment of B rings relates to B rings that are a phenyl ring substituted with from 1 to 5 organic radicals chosen from:

i) methyl, ethyl, n-propyl, iso-propyl, cyclopropyl, or tert-butyl;

ii) —OH, —CH₂OH, —OCH₃, —CH₂OCH₃, or —OCH₂CH₃;

iii) —COCH₃;

iv) —CO₂CH₃, —CH₂CO₂CH₃, or —CO₂CH₂CH₃;

v) —CONH₂, —CONHCH₃, or —CON(CH₃)₂;

vi) —NH₂, —NHCH₃, or —N(CH₃)₂;

vii) —F, —Cl, —Br, and —I;

viii) —CF₃;

ix) —CN;

x) —NO₂; and

xi) —SO₂CH₃ or —SO₂C₆H₅.

The following are non-limiting examples of substituted phenyl:

Halogen substituted phenyl, including 2-fluorophenyl, 3-fluorophenyl, 4-fluorophenyl, 2,3-difluorophenyl, 2,4-difluorophenyl, 2,5-difluorophenyl, 2,6-difluorophenyl, 3,4-difluorophenyl, 3,5-difluorophenyl, 2,3,4-trifluorophenyl, 2,3,5-trifluorophenyl, 2,3,6-trifluorophenyl, 2,4,6-trifluorophenyl, 2,3,4,5-tetrafluorophenyl, 2,3,4,6-tetrafluorophenyl, 2,3,4,5,6-pentafluorophenyl, 2-chlorophenyl, 3-chlorophenyl, 4-chlorophenyl, 2,3-dichlorophenyl, 2,4-dichlorophenyl, 2,5-dichlorophenyl, 2,6-dichlorophenyl, 3,4-dichlorophenyl, 3,5-dichlorophenyl, 2,3,4-trichlorophenyl, 2,3,5-trichlorophenyl, 2,3,6-trichlorophenyl, 2,4,6-trichlorophenyl, 2,3,4,5-tetrachlorophenyl, 2,3,4,6-tetrachlorophenyl, 2,3,4,5,6-pentachlorophenyl, 2-bromophenyl, 3-bromophenyl, 4-bromophenyl, 2,3-dibromophenyl, 2,4-dibromophenyl, 2,5-dibromophenyl, 2, 6-dibromophenyl, 3,4-dibromophenyl, 3,5-dibromophenyl, 2,3,4-tribromophenyl, 2,3,5-tribromophenyl, 2,3,6-tribromophenyl, 2,4,6-tribromophenyl, 2,3,4,5-tetrabromophenyl, 2,3,4,6-tetrabromophenyl, and 2,3,4,5,6-pentabromophenyl.

Hydroxy and alkoxy substituted phenyl, including 2-hydroxyphenyl, 3-hydroxyphenyl, 4-hydroxyphenyl, 2,3-dihydroxyphenyl, 2,4-dihydroxyphenyl, 2,5-dihydroxyphenyl, 2,6-dihydroxyphenyl, 3,4-dihydroxyphenyl, 3,5-dihydroxyphenyl, 2,3,4-trihydroxyphenyl, 2,3,5-trihydroxyphenyl, 2,3,6-trihydroxyphenyl, 2,4,6-trihydroxyphenyl, 2,3,4,5-tetrahydroxyphenyl, 2,3,4,6-tetrahydroxyphenyl, 2,3,4,5,6-pentahydroxyphenyl, 2-methoxyphenyl, 3-methoxyphenyl, 4-methoxyphenyl, 2,3-dimethoxyphenyl, 2,4-dimethoxyphenyl, 2,5-dimethoxyphenyl, 2,6-dimethoxyphenyl, 3,4-dimethoxyphenyl, 3,5-dimethoxyphenyl, 2,3,4-trimethoxyphenyl, 2,3,5-trimethoxyphenyl, 2,3,6-trimethoxyphenyl, 2,4,6-trimethoxyphenyl, 2,3,4,5-tetramethoxyphenyl, 2,3,4,6-tetramethoxyphenyl, 2,3,4,5,6-pentamethoxyphenyl, 2-ethoxyphenyl, 3-ethoxyphenyl, 4-ethoxyphenyl, 2,3-diethoxyphenyl, 2,4-diethoxyphenyl, 2,5-diethoxyphenyl, 2,6-diethoxyphenyl, 3,4-diethoxyphenyl, 3,5-diethoxyphenyl, 2,3,4-triethoxyphenyl, 2,3,5-triethoxyphenyl, 2,3,6-triethoxyphenyl, 2,4,6-triethoxyphenyl, 2,3,4,5-tetraethoxyphenyl, 2,3,4,6-tetraethoxyphenyl, and 2,3,4,5,6-pentaethoxyphenyl.

Alkyl substituted phenyl, including 2-methylphenyl, 3-methylphenyl, 4-methylphenyl, 2,3-dimethylphenyl, 2,4-dimethylphenyl, 2,5-dimethylphenyl, 2,6-dimethylphenyl, 3,4-dimethylphenyl, 3,5-dimethylphenyl, 2,3,4-trimethylphenyl, 2,3,5-trimethylphenyl, 2,3,6-trimethylphenyl, 2,4,6-trimethylphenyl, 2,3,4,5-tetramethylphenyl, 2,3,4,6-tetramethylphenyl, 2,3,4,5,6-pentamethylphenyl, 2-ethylphenyl, 3-ethylphenyl, 4-ethylphenyl, 2,3-diethylphenyl, 2,4-diethylphenyl, 2,5-diethylphenyl, 2,6-diethylphenyl, 3,4-diethylphenyl, 3,5-diethylphenyl, 2,3,4-triethylphenyl, 2,3,5-triethylphenyl, 2,3,6-triethylphenyl, 2,4,6-triethylphenyl, 2,3,4,5-tetraethylphenyl, 2,3,4,6-tetraethylphenyl, and 2,3,4,5,6-pentaethylphenyl.

Haloalkyl and nitro substituted phenyl, including 2-(trifluoromethyl)phenyl, 3-(trifluoromethyl)phenyl, 4-(trifluoromethyl)phenyl, 2,3-di(trifluoromethyl)phenyl, 2,4-di(trifluoromethyl)phenyl, 2,5-di(trifluoromethyl)phenyl, 2,6-di(trifluoromethyl)-phenyl, 3,4-di(trifluoromethyl)phenyl, 3,5-di(trifluoromethyl)phenyl, 2,3,4-tri(trifluoro-methyl)phenyl, 2,3,5-tri(trifluoromethyl)phenyl, 2,3,6-tri(trifluoromethyl)phenyl, 2,4,6-tri(trifluoromethyl)phenyl, 2,3,4,5-tetra(trifluoromethyl)phenyl, 2,3,4,6-tetra(trifluoro-methyl)phenyl, 2,3,4,5,6-penta(trifluoromethyl)phenyl, 2-nitrophenyl, 3-nitrophenyl, 4-nitrophenyl, 2,3-dinitrophenyl, 2,4-dinitrophenyl, 2,5-dinitrophenyl, 2,6-dinitrophenyl, 3,4-dinitrophenyl, 3,5-dinitrophenyl, 2,3,4-trinitrophenyl, 2,3,5-trinitrophenyl, 2,3,6-trinitrophenyl, 2,4,6-trinitrophenyl, 2,3,4,5-tetranitrophenyl, 2,3,4,6-tetranitrophenyl, and 2,3,4,5,6-pentanitrophenyl.

L is a linking unit that can be optionally present. When the index x is equal to 0, then L is absent. When the index x is equal to 1, then L is present.

L is a linking unit having in the chain from 1 to 6 carbon atoms or from 1 to 5 carbon atoms together with from 1 to 4 heteroatoms chosen from nitrogen, oxygen, or sulfur.

The first aspect of L relates to alkylene units having the formula:

—[C(R^{6a}R^{6b})]_{w}—

wherein R^{6a} and R^{6b} are each independently chosen from hydrogen or methyl, and the index w is from 1 to 6. Non-limiting examples of this aspect of L include:

i) —CH₂CH₂—;

ii) —CH₂CH₂CH₂—;

iii) —CH₂CH₂CH₂CH₂—;

iv) —CH₂CH(CH₃)CH₂—;

v) —CH₂CH(CH₃)CH₂CH₂—;

vi) —CH₂CH₂CH(CH₃)CH₂—; and

vii) —CH₂CH₂CH₂CH₂CH₂CH₂—.

The second aspect of L includes units comprising from 1 to 5 carbon atoms and one or more heteroatoms chosen from nitrogen, oxygen, or sulfur. Non-limiting examples include:

i) —NHCH₂CH₂—;

ii) —NHC(O)CH₂CH₂—;

iii) —CH₂C(O)NHCH₂—;

iv) —CH(CH₃)C(O)NHCH₂—;

v) —CH₂C(O)NHCH(CH₃)—;

vi) —CH(CH₃)C(O)NHCH(CH₃)—;

v) —CH₂OCH₂CH₂—; and

v) —CH₂SCH₂CH₂—.

L is a linking unit that can be optionally present. When the index x is equal to 0, then L is absent. When the index x is equal to 1, then L is present.

L¹ is a linking unit having in the chain from 1 to 6 carbon atoms or from 1 to 5 carbon atoms together with from 1 to 4 heteroatoms chosen from nitrogen, oxygen, or sulfur

The first aspect of L¹ relates to alkylene units having the formula:

—[C(R^{15a}R^{15b})]_{z}—

wherein R^{15a} and R^{15b} are each independently chosen from hydrogen or methyl, and the index z is from 1 to 6. Non-limiting examples of this aspect of L¹ include:

i) —CH₂CH₂—;

ii) —CH₂CH₂CH₂—;

iii) —CH₂CH₂CH₂CH₂—;

iv) —CH₂CH(CH₃)CH₂—;

v) —CH₂CH(CH₃)CH₂CH₂—;

vi) —CH₂CH₂CH(CH₃)CH₂—; and

vii) —CH₂CH₂CH₂CH₂CH₂CH₂—.

The second aspect of L¹ includes units comprising from 1 to 5 carbon atoms and one or more heteroatoms chosen from nitrogen, oxygen, or sulfur. Non-limiting examples include:

i) —CH₂S—;

ii) —CH(CH₃)S—;

ii) —CH₂SCH₂CH₂—;

iv) —CH(CH₃)SCH₂CH₂—;

v) —CH₂O—;

vi) —CH(CH₃)O—;

vii) —CH₂OCH₂CH₂—;

viii) —CH(CH₃)OCH₂CH₂—; and

ix) —CH₂CH₂OCH₂CH₂O—.

A first aspect of this category relates to compounds having the formula:

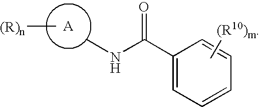

A first embodiment of this aspect relates to compounds comprising an unsubstituted A ring. The compounds of this embodiment can be prepared by coupling a substituted or unsubstituted 5-member ring heteroaryl unit with a substituted or unsubstituted carboxylic acid, for example, as depicted in Scheme I below.

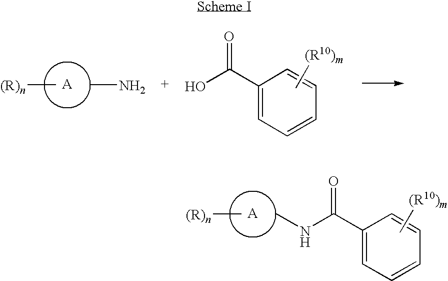

Reagents and conditions: EDCI, NMM, HOBt, DMF; rt.

However, the artisan can use any coupling procedure, inter alia, forming the acid chloride of the corresponding carboxylic acid, or any other coupling reagents to achieve the desire amide. A variety of heteroaryl amines are commercially available. In addition, many substituted aryl carboxylic acids are also available.

One example of this embodiment is unsubstituted heteroaryl benzamides, for example, 2,4,5-trimethoxy-N-(1H-1,2,4-triazol-3-yl)benzamide having the formula:

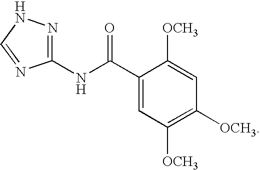

Preparation of 2,4,5-trimethoxy-N-(1H-1,2,4-triazol-3-yl)benzamide: 2,4,5-trimethoxybenzoyl chloride (2.3 g, 10 mmol) in THF (25 mL) is cooled to 0° C. in an ice bath. A solution of 3-amino-1H-1,2,4-triazole (0.924 g, 11 mmol) in THF (10 mL) is added dropwise to the solution of acid chloride over approximately 30 minutes. The cooling bath is removed and the solution is allowed to continue stirring as it warms to room temperature. After approximately 1 hour at room temperature, the contents of the reaction flask is poured into CH₂Cl₂ (100 mL). The solution is extracted twice with 0.1 N HCl (10 mL), water (25 mL), brine (25 mL) and dried over Na₂SO₄. The solvent is removed in vacuo to afford the desired product.

Another embodiment of this aspect relates to compounds having a substituted A ring. A non-limiting example of compounds according to this embodiment is N-[1-(2-chloro-4-methoxyphenyl)-5-methyl-1H-1,2,4-triazol-3-yl)-2,4,5-trimethoxy-benzamide having the formula:

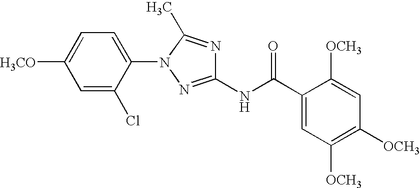

Compounds according to this embodiment can be prepared according to the example provide in Scheme II. N-[1-(2-Chloro-4-methoxyphenyl)-5-methyl-1H-1,2,4-triazol-3-yl)-2,4,5-trimethoxy-benzamide can be prepared from Intermediate 3 by coupling this intermediate with 2,4,5-trimethoxybenzoic acid the procedure described herein above. The preparation of Intermediate 3 is outlined in Scheme II.

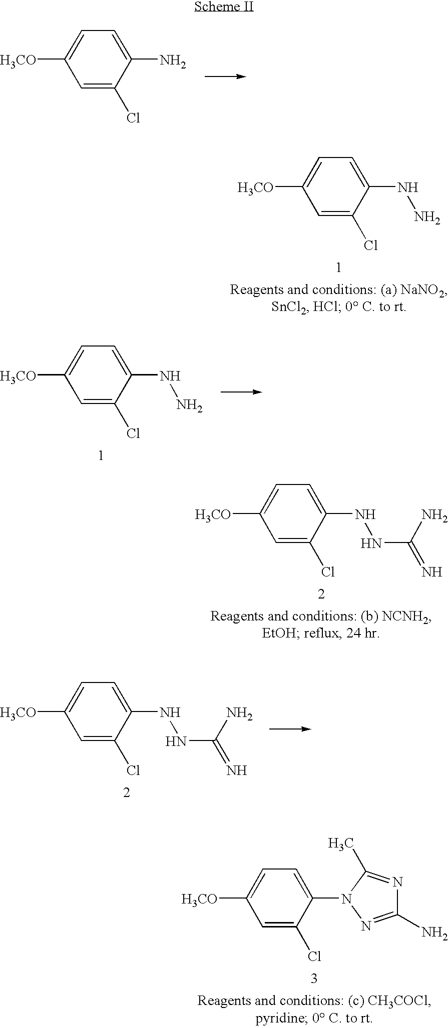

Preparation of 2-chloro-4-methoxy-5-methylphenylhydrazine HCl (1): A solution of 8.6 g (50 mmol) of 2-chloro-4-methoxy-5-methylaniline in 75 ml of 5N HCl is stirred at −5° C. and 3.52 g (51 mmol) of sodium nitrite, in solution in 12.5 mL of water, are added. The mixture is stirred for one hour at 0° C. and then a solution of 22.56 g (100 mmol) of tin(II) chloride dihydrate in 20 mL of 35% hydrochloric acid is added. The mixture is stirred for two hours with gradual return to ambient temperature. The precipitate that forms is filtered off and washed with 1N HCl, ethanol and diethyl ether. The isolated precipitate is then dried to a constant weight. The reported M.p. for 2-chloro-4-methoxy-5-methylphenylhydrazine HCl is 140° C.

Preparation of 2-(2-chloro-4-methoxyphenyl)hydrazinecarboximidamide HCl (2): A mixture of 2-chloro-4-methoxy-5-methylphenylhydrazine HCl, 1, (5 g, 24 mmol) and cyanamide (1.26 g, 30 mmol) is refluxed in absolute ethanol for 24 hours. The reaction is cooled and diethyl ether is added. The precipitate that forms is collected by filtration. The crude product can be used without further purification or recrystallized, for example, dissolved in hot alcohol and treated with diethyl ether.

Preparation of 1-(2-chloro-4-methoxyphenyl)-3-amino-5-methyl-1H-1,2,4-triazole (3): A suspension of 2-(2-chloro-4-methoxyphenyl)hydrazinecarboximidamide HCl, 2, (0.72 g, 3 mmol) in pyridine (12 mL) is cooled to 0° C. and acetyl chloride (15 mmol) is slowly added. The reaction mixture is stirred overnight at room temperature and is then poured into ice water (100 mL). The solution is acidified to pH 1 with 1N HCl, and the mixture is then extracted with ethyl acetate. The organic phase is washed with NaHCO₃, water, then with brine. The solution is dried over Na₂SO₄ then concentrated to afford the desired product that can be used without purification or purified by re-crystallization or by chromatography.

The following are non-limiting examples of compounds according to this aspect of the disclosed tissue-non-specific alkaline phosphatase activators:

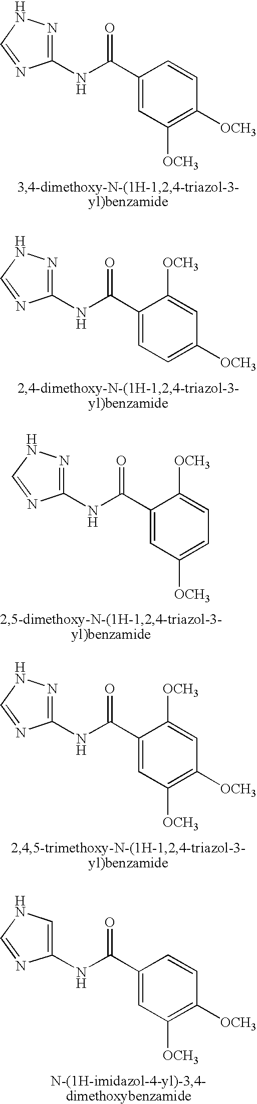

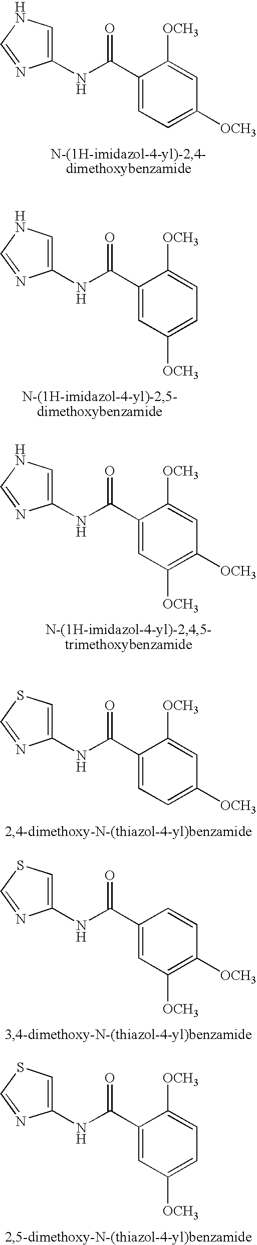

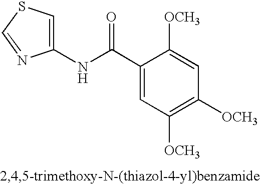

Another aspect of this category of tissue-nonspecific alkaline phosphatase activators of the present disclosure are amides having the formula:

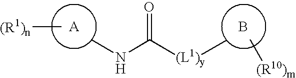

wherein A, L, L¹, n, x and y are the same as defined herein above and B represents a phenyl ring, a 5-member or 6-member cycloalkyl or a heterocyclic ring as defined herein above that can optionally have from 1 to 5 hydrogen atoms substituted by an organic radical R¹⁰.

One embodiment relates to compounds having the formula:

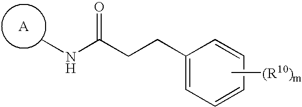

wherein A is a 5-member ring heteroaryl ring and R¹⁰ represents from 1 to 5 organic radical optionally present.

Compounds of this embodiment can be prepared according to Scheme III using the coupling procedures

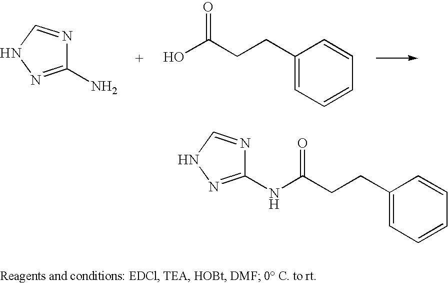

Preparation of 3-phenyl-N-(1H-1,2,4-triazol-3-yl)propanamide: To a solution of 3-amino-1H-1,2,4-triazole HCl (0.13 g, 1.1 mmol), 3-phenyl propanoic acid (0.21 g, 1.4 mmol) and 1-hydroxybenzotriazole (HOBt) (0.094 g, 0.70 mmol) in DMF (10 mL) at 0° C., is added 1-(3-dimethylaminopropyl)-3-ethylcarbodiimide (EDCI) (0.268 g, 1.4 mmol) followed by triethylamine (0.60 mL, 4.2 mmol). The mixture is stirred at 0° C. for 30 minutes then at room temperature overnight. The reaction mixture is diluted with water and extracted with EtOAc. The combined organic phase is washed with 1 N aqueous HCl, 5% aqueous NaHCO₃, water and brine, and dried over Na₂SO₄. The solvent is removed in vacuo to afford the desired product.

Another embodiment relates to compounds having the formula:

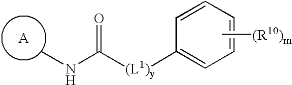

wherein A is a 5-member ring heteroaryl ring, R¹⁰ represents from 1 to 5 organic radicals optionally present, and L¹ is a linking group comprising from 1 to 5 carbon atoms together with one or more heteroatoms chosen from nitrogen, oxygen, or sulfur.

A further embodiment relates to compounds having the formula:

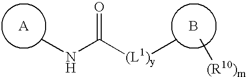

wherein A is a substituted or unsubstituted 5-member ring heteroaryl ring, B is a substituted or unsubstituted cyclopentyl or cyclohexyl, R¹⁰ represents from 1 to 5 organic radicals optionally present, and L¹ is a linking group comprising from 1 to 5 carbon atoms together with one or more heteroatoms chosen from nitrogen, oxygen, or sulfur.

A yet further embodiment relates to compounds having the formula:

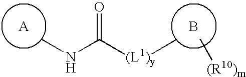

wherein A is a substituted or unsubstituted 5-member ring heteroaryl ring, B is a substituted or unsubstituted 5-member heterocyclic ring, R¹⁰ represents from 1 to 5 organic radicals optionally present, and L¹ is a linking group comprising from 1 to 6 carbon atoms.

The following are non-limiting examples of compounds according to this aspect of the disclosed tissue-non-specific alkaline phosphatase activators:

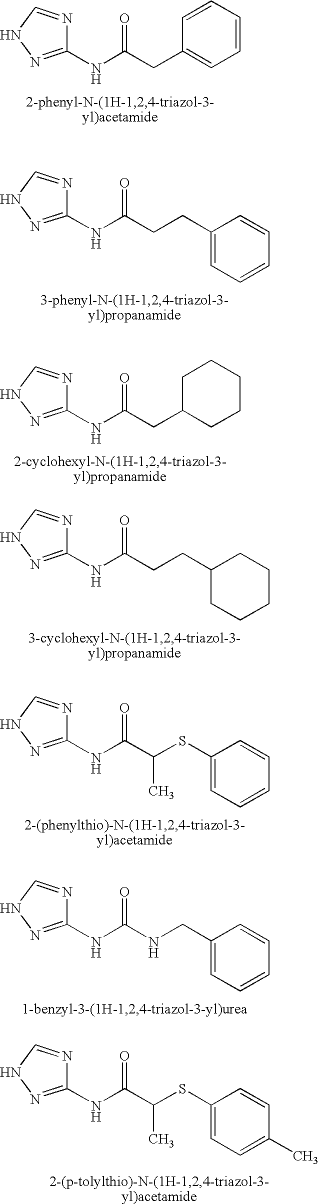

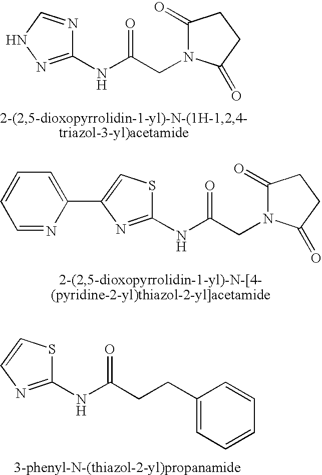

The following Table 1 provides examples of the disclosed tissue-nonspecific alkaline phosphatase (TNAP) activators according to this category.

TABLE 1
TNAP activators

| | TNAP activation |
|---|---|
| Compound | factor |
| 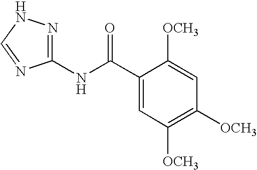 | 15.4 |
| 2,4,5-trimethoxy-N-(1H-1,2,4-triazol-3-yl)benzamide | |
| 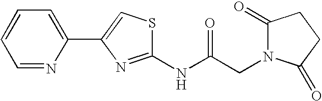 | 2.0 |
| 2-(2,5-dioxopyrrolidin-1-yl)-N-[4-(pyridine-2- | |
| yl)thiazol-2-yl]acetamide | |
| 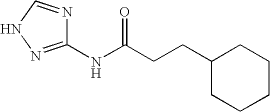 | 1.8 |
| 3-cyclohexyl-N-(1H-1,2,4-triazol-3-yl)propanamide | |
| 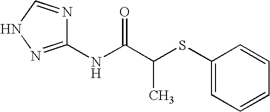 | 1.6 |
| 2-(phenylthio)-N-(1H-1,2,4-triazol-3-yl)acetamide | |
| 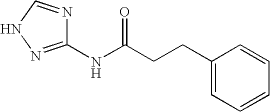 | 1.5 |
| 3-phenyl-N-(1H-1,2,4-triazol-3-yl)propanamide | |

The second category of tissue-nonspecific alkaline phosphatase activators of the present disclosure are amines having the formula

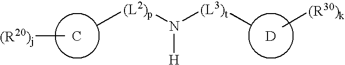

wherein C represents a substituted or unsubstituted heterocyclic or heteroaryl ring comprising from 5 to 10 carbon atoms and from 1 to 4 heteroatoms independently chosen from oxygen, nitrogen, and sulfur. D represents a substituted or unsubstituted heterocyclic or heteroaryl ring comprising from 5 to 10 carbon atoms and from 1 to 4 heteroatoms independently chosen from oxygen, nitrogen, and sulfur.

L² is a linking unit that can be optionally present. When the index p is equal to 0, then L is absent. When the index p is equal to 1, then L is present.

L² is a linking unit comprising from 1 to 6 carbon atoms or from 1 to 5 carbon atoms together with one or more heteroatoms chosen from nitrogen, oxygen, or sulfur. The first aspect of L relates to alkylene units having the formula:

—[C(R2^{6a}R2^{6b})]ₛ—

wherein R^{26a} and R^{26b} are each independently chosen from hydrogen or methyl, and the index s is from 1 to 6. Non-limiting examples of this aspect of L² include:

i) —CH₂CH₂—;

ii) —CH₂CH₂CH₂—;

iii) —CH₂CH₂CH₂CH₂—;

iv) —CH₂CH(CH₃)CH₂—;

v) —CH₂CH(CH₃)CH₂CH₂—;

vi) —CH₂CH₂CH(CH₃)CH₂—; and

vii) —CH₂CH₂CH₂CH₂CH₂CH₂—.

The second aspect of L² includes units comprising from 1 to 5 carbon atoms and one or more heteroatoms chosen from nitrogen, oxygen, or sulfur. Non-limiting examples include:

i) —NHCH₂CH₂—;

ii) —NHC(O)CH₂CH₂—;

iii) —CH₂C(O)NHCH₂—;

iv) —CH(CH₃)C(O)NHCH₂—;

v) —CH₂C(O)NHCH(CH₃)—;

vi) —CH(CH₃)C(O)NHCH(CH₃)—;

vii) —CH₂OCH₂CH₂—; and

viii) —CH₂SCH₂CH₂—.

L³ is a linking unit that can be optionally present. When the index t is equal to 0, then L³ is absent. When the index t is equal to 1, then L² is present.

L³ is a linking unit comprising from 1 to 6 carbon atoms or from 1 to 5 carbon atoms together with one or more heteroatoms chosen from nitrogen, oxygen, or sulfur. The first aspect of L³ relates to alkylene units having the formula:

—[C(R^{35a}R^{35b})]ᵣ—

wherein R^{35a} and R^{35b} are each independently chosen from hydrogen or methyl, and the index r is from 1 to 6. Non-limiting examples of this aspect of L³ include:

i) —CH₂CH₂—;

ii) —CH₂CH₂CH₂—;

iii) —CH₂CH₂CH₂CH₂—;

iv) —CH₂CH(CH₃)CH₂—;

v) —CH₂CH(CH₃)CH₂CH₂—;

vi) —CH₂CH₂CH(CH₃)CH₂—; and

vii) —CH₂CH₂CH₂CH₂CH₂CH₂—.

The second aspect of L³ includes units comprising from 1 to 5 carbon atoms and one or more heteroatoms chosen from nitrogen, oxygen, or sulfur. Non-limiting examples include:

i) —CH₂S—;

ii) —CH(CH₃)S—;

ii) —CH₂SCH₂CH₂—;

iv) —CH(CH₃)SCH₂CH₂—;

v) —CH₂O—;

yl) —CH(CH₃)O—;

vii) —CH₂OCH₂CH₂—;

viii) —CH(CH₃)OCH₂CH₂—; and

ix) —CH₂CH₂OCH₂CH₂O—.

The first embodiment of the disclosed compounds according to this category have the formula:

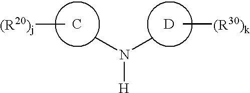

wherein C is a substituted or unsubstituted 5-member heteroaryl ring. D is a substituted or unsubstituted 6-member heteroaryl ring.

C units can comprise a substituted or unsubstituted 5-member heteroaryl ring. The following are non-limiting examples of 5-member heteroaryl rings:

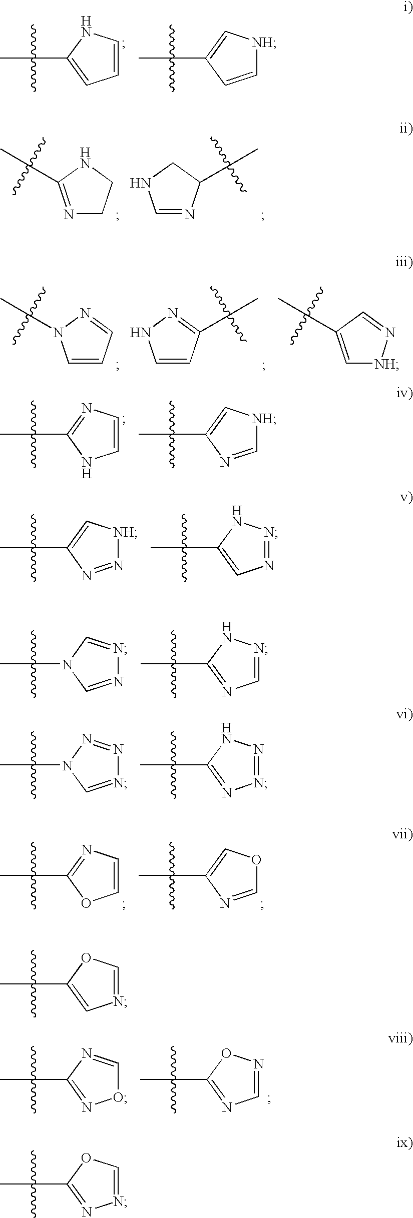

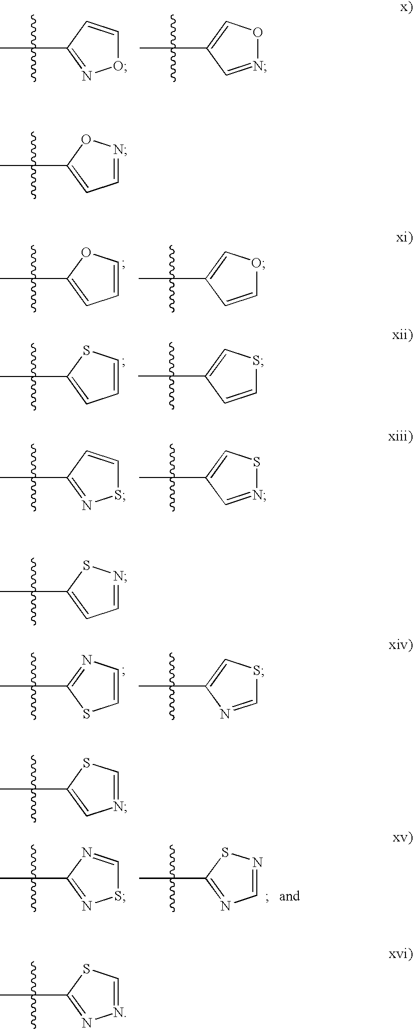

The individual R²⁰ organic radicals are each independently chosen from one another. The following are non-limiting examples of organic radicals that can substitute for a hydrogen atom of the C ring:
i) linear, branched, or cyclic alkyl, alkenyl, and alkynyl; for example, methyl (C₁), ethyl (C₂), n-propyl (C₃), iso-propyl (C₃), cyclopropyl (C₃), propylen-2-yl (C₃), propargyl (C₃), n-butyl (C₄), iso-butyl (C₄), sec-butyl (C₄), tert-butyl (C₄), cyclobutyl (C₄), n-pentyl (C₅), cyclopentyl (C₅), n-hexyl (C₆), and cyclohexyl (C₆);
ii) substituted or unsubstituted aryl; for example, phenyl, 2-fluorophenyl, 3-chlorophenyl, 4-methylphenyl, 2-aminophenyl, 3-hydroxyphenyl, 4-trifluoromethylphenyl, and biphenyl-4-yl;
iii) substituted or unsubstituted heterocyclic; for example, piperidinyl, pyrrolidinyl, and morpholinyl;
iv) substituted or unsubstituted heteroaryl; for example, pyrrolyl, pyridinyl, and pyrimidinyl;
v) —(CR^{23a}R^{23b})_{q}OR²²; for example, —OH, —CH₂OH, —OCH₃, —CH₂OCH₃, —OCH₂CH₃, —CH₂OCH₂CH₃, —OCH₂CH₂CH₃, and —CH₂OCH₂CH₂CH₃;
vi) —(CR^{23a}R^{23b})_{q}C(O)R²²; for example, —COCH₃, —CH₂COCH₃, —OCH₂CH₃,
   —CH₂COCH₂CH₃, —COCH₂CH₂CH₃, and —CH₂COCH₂CH₂CH₃;
vii) —(CR^{23a}R^{23b})_{q}C(O)OR²²; for example, —CO₂CH₃, —CH₂CO₂CH₃, —CO₂CH₂CH₃, —CH₂CO₂CH₂CH₃, —CO₂CH₂CH₂CH₃, and —CH₂CO₂CH₂CH₂CH₃;
viii) —(CR^{23a}R^{23b})_{q}C(O)N(R²²)₂; for example, —CONH₂, —CH₂CONH₂, —CONHCH₃, —CH₂CONHCH₃, —CON(CH₃)₂, and —CH₂CON(CH₃)₂;
ix) —(CR^{23a}R^{23b})_{q}OC(O)N(R²²)₂; for example, —OC(O)NH₂, —CH₂OC(O)NH₂, —OC(O)NHCH₃, —CH₂OC(O)NHCH₃, —OC(O)N(CH₃)₂, and —CH₂OC(O)N(CH₃)₂;
x) —(CR^{23a}R^{23b})_{q}N(R²²)₂; for example, —NH₂, —CH₂NH₂, —NHCH₃, —N(CH₃)₂, —NH(CH₂CH₃), —CH₂NHCH₃, —CH₂N(CH₃)₂, and —CH₂NH(CH₂CH₃);
xi) halogen: —F, —Cl, —Br, and —I;
xii) —CHₘXₙ; wherein X is halogen, m is from 0 to 2, m+n=3; for example, —CH₂F, —CHF₂, —CF₃, —CCl₃, or —CBr₃;
xiii) —(CR^{23a}R^{23b})_{q}CN; for example; —CN, —CH₂CN, and —CH₂CH₂CN;
xiv) —(CR^{23a}R^{23b})_{q}NO₂; for example; —NO₂, —CH₂NO₂, and —CH₂CH₂NO₂;
xv) —(CR^{23a}R^{23b})_{q}SO₂R²²; for example, —SO₂H, —CH₂SO₂H, —SO₂CH₃, —CH₂SO₂CH₃, —SO₂C₆H₅, and —CH₂SO₂C₆H₅; and
xvi) —(CR^{23a}R^{23b})_{q}SO₃R²²; for example, —SO₃H, —CH₂SO₃H, —SO₃CH₃, —CH₂SO₃CH₃, —SO₃C₆H₅, and —CH₂SO₃C₆H₅;
   wherein each R²² is independently hydrogen, substituted or unsubstituted C₁-C₄ linear, branched, or cyclic alkyl; or two R²² units can be taken together to form a ring comprising 3-7 atoms; R^{23a} and R^{23b} are each independently hydrogen or C₁-C₄ linear or branched alkyl; the index q is from 0 to 4.

When R²⁰ comprises C₁-C₁₂ linear, branched, or cyclic alkyl, alkenyl; substituted or unsubstituted C₆ or C₁₀aryl; substituted or unsubstituted C₁-C₉heterocyclic; or substituted or unsubstituted C₁-C₉heteroaryl; R²⁰ can further have one or more hydrogen atoms substituted by one or more organic radicals. Non-limiting examples of organic radicals that can substitute for a hydrogen atom of R²⁰ include:
i) linear, branched, or cyclic alkyl, alkenyl, and alkynyl; for example, methyl (C₁), ethyl (C₂), n-propyl (C₃), iso-propyl (C₃), cyclopropyl (C₃), propylen-2-yl (C₃), propargyl (C₃), n-butyl (C₄), iso-butyl (C₄), sec-butyl (C₄), tert-butyl (C₄), cyclobutyl (C₄), n-pentyl (C₅), cyclopentyl (C₅), n-hexyl (C₆), and cyclohexyl (C₆);
ii) —(CR^{25a}R^{25b})_{q}OR²⁴; for example, —OH, —CH₂OH, —OCH₃, —CH₂OCH₃, —OCH₂CH₃, —CH₂OCH₂CH₃, —OCH₂CH₂CH₃, and —CH₂OCH₂CH₂CH₃;
iii) —(CR^{25a}R^{25b})_{q}C(O)R²⁴; for example, —COCH₃, —CH₂COCH₃, —OCH₂CH₃, —CH₂COCH₂CH₃, —COCH₂CH₂CH₃, and —CH₂COCH₂CH₂CH₃;
iv) —(CR^{25a}R^{25b})_{q}C(O)OR²⁴; for example, —CO₂CH₃, —CH₂CO₂CH₃, —CO₂CH₂CH₃, —CH₂CO₂CH₂CH₃, —CO₂CH₂CH₂CH₃, and —CH₂CO₂CH₂CH₂CH₃;
v) —(CR^{25a}R^{25b})_{q}C(O)N(R²⁴)₂; for example, —CONH₂, —CH₂CONH₂, —CONHCH₃, —CH₂CONHCH₃, —CON(CH₃)₂, and —CH₂CON(CH₃)₂;
vi) —(CR^{25a}R^{25a})_{q}OC(O)N(R²⁴)₂; for example, —OC(O)NH₂, —CH₂OC(O)NH₂, —OC(O)NHCH₃, —CH₂OC(O)NHCH₃, —OC(O)N(CH₃)₂, and —CH₂OC(O)N(CH₃)₂;
vii) —(CR^{25a}R^{25b})_{q}N(R²⁴)₂; for example, —NH₂, —CH₂NH₂, —NHCH₃, —N(CH₃)₂, —NH(CH₂CH₃), —CH₂NHCH₃, —CH₂N(CH₃)₂, and —CH₂NH(CH₂CH₃);
viii) halogen: —F, —Cl, —Br, and —I;
ix) —CHₘXₙ; wherein X is halogen, m is from 0 to 2, m+n=3; for example, —CH₂F, —CHF₂, —CF₃, —CCl₃, or —CBr₃;
x) —(CR^{25a}R^{25b})_{q}CN; for example; —CN, —CH₂CN, and —CH₂CH₂CN;
xi) —(CR^{25a}R^{25b})_{q}NO₂; for example; —NO₂, —CH₂NO₂, and —CH₂CH₂NO₂;
xii) —(CR^{25a}R^{25b})_{q}SO₂R²⁴; for example, —SO₂H, —CH₂SO₂H, —SO₂CH₃, —CH₂SO₂CH₃, —SO₂C₆H₅, and —CH₂SO₂C₆H₅; and
xiii) —(CR^{25a}R^{25b})_{q}SO₃R²⁴; for example, —SO₃H, —CH₂SO₃H, —SO₃CH₃, —CH₂SO₃CH₃, —SO₃C₆H₅, and —CH₂SO₃C₆H₅;
   wherein each R²⁴ is independently hydrogen, substituted or unsubstituted C₁-C₄ linear, branched, or cyclic alkyl; or two R²⁴ units can be taken together to form a ring comprising 3-7 atoms; R^{25a} and R^{25b} are each independently hydrogen or C₁-C₄ linear or branched alkyl; the index p is from 0 to 4.

D rings are substituted or unsubstituted 6-member heteroaryl rings. Non-limiting examples of D rings include:

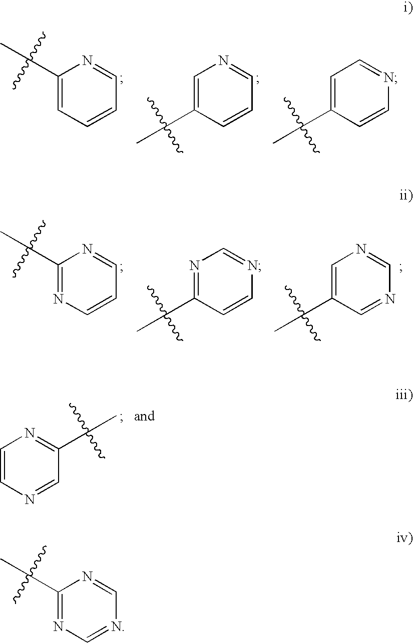

The individual R³⁰ organic radicals are each independently chosen from one another. The following are non-limiting examples of organic radicals that can substitute for a hydrogen atom of a D ring:
i) linear, branched, or cyclic alkyl, alkenyl, and alkynyl; for example, methyl (C₁), ethyl (C₂), n-propyl (C₃), iso-propyl (C₃), cyclopropyl (C₃), propylen-2-yl (C₃), propargyl (C₃), n-butyl (C₄), iso-butyl (C₄), sec-butyl (C₄), tert-butyl (C₄), cyclobutyl (C₄), n-pentyl (C₅), cyclopentyl (C₅), n-hexyl (C₆), and cyclohexyl (C₆);
ii) substituted or unsubstituted aryl; for example, phenyl, 2-fluorophenyl, 3-chlorophenyl, 4-methylphenyl, 2-aminophenyl, 3-hydroxyphenyl, 4-trifluoromethylphenyl, and biphenyl-4-yl;
iii) substituted or unsubstituted heterocyclic; for example, piperidinyl, pyrrolidinyl, and morpholinyl;
iv) substituted or unsubstituted heteroaryl; for example, pyrrolyl, pyridinyl, and pyrimidinyl;
v) —(CR^{33a}R^{33b})_{q}OR³²; for example, —OH, —CH₂OH, —OCH₃, —CH₂OCH₃, —OCH₂CH₃, —CH₂OCH₂CH₃, —OCH₂CH₂CH₃, and —CH₂OCH₂CH₂CH₃;
vi) —(CR^{33a}R^{33b})_{q}C(O)R³²; for example, —COCH₃, —CH₂COCH₃, —OCH₂CH₃,
   —CH₂COCH₂CH₃, —COCH₂CH₂CH₃, and —CH₂COCH₂CH₂CH₃;
vii) —(CR^{33a}R^{33b})_{q}C(O)OR³²; for example, —CO₂CH₃, —CH₂CO₂CH₃, —CO₂CH₂CH₃, —CH₂CO₂CH₂CH₃, —CO₂CH₂CH₂CH₃, and —CH₂CO₂CH₂CH₂CH₃;
viii) —(CR^{33a}R^{33b})_{q}C(O)N(R³²)₂; for example, —CONH₂, —CH₂CONH₂, —CONHCH₃, —CH₂CONHCH₃, —CON(CH₃)₂, and —CH₂CON(CH₃)₂;
ix) —(CR^{33a}R^{33b})_{q}OC(O)N(R³²)₂; for example, —OC(O)NH₂, —CH₂OC(O)NH₂, —OC(O)NHCH₃, —CH₂OC(O)NHCH₃, —OC(O)N(CH₃)₂, and —CH₂OC(O)N(CH₃)₂;
x) —(CR^{33a}R^{33b})_{q}N(R³²)₂; for example, —NH₂, —CH₂NH₂, —NHCH₃, —N(CH₃)₂, —NH(CH₂CH₃), —CH₂NHCH₃, —CH₂N(CH₃)₂, and —CH₂NH(CH₂CH₃);
xi) halogen: —F, —Cl, —Br, and —I;
xii) —CHₘXₙ; wherein X is halogen, m is from 0 to 2, m+n=3; for example, —CH₂F, —CHF₂, —CF₃, —CCl₃, or —CBr₃;
xiii) —(CR^{33a}R^{33b})_{q}CN; for example; —CN, —CH₂CN, and —CH₂CH₂CN;
xiv) —(CR^{33a}R^{33b})_{q}NO₂; for example; —NO₂, —CH₂NO₂, and —CH₂CH₂NO₂;
xv) —(CR^{33a}R^{33b})_{q}SO₂R³²; for example, —SO₂H, —CH₂SO₂H, —SO₂CH₃, —CH₂SO₂CH₃, —SO₂C₆H₅, and —CH₂SO₂C₆H₅; and
xvi) —(CR^{33a}R^{33b})_{q}SO₃R³²; for example, —SO₃H, —CH₂SO₃H, —SO₃CH₃, —CH₂SO₃CH₃, —SO₃C₆H₅, and —CH₂SO₃C₆H₅;
   wherein each R³² is independently hydrogen, substituted or unsubstituted C₁-C₄ linear, branched, or cyclic alkyl; or two R³² units can be taken together to form a ring comprising 3-7 atoms; R^{33a} and R^{33b} are each independently hydrogen or C₁-C₄ linear or branched alkyl; the index q is from 0 to 4.

When R³⁰ comprises C₁-C₁₂ linear, branched, or cyclic alkyl, alkenyl; substituted or unsubstituted C₆ or C₁₀ aryl; substituted or unsubstituted C₁-C₉heterocyclic; or substituted or unsubstituted C₁-C₉heteroaryl; R³⁰ can further have one or more hydrogen atoms substituted by one or more organic radicals. Non-limiting examples of organic radicals that can substitute for a hydrogen atom of R³⁰ include:
i) linear, branched, or cyclic alkyl, alkenyl, and alkynyl; for example, methyl (C₁), ethyl (C₂), n-propyl (C₃), iso-propyl (C₃), cyclopropyl (C₃), propylen-2-yl (C₃), propargyl (C₃), n-butyl (C₄), iso-butyl (C₄), sec-butyl (C₄), tert-butyl (C₄), cyclobutyl (C₄), n-pentyl (C₅), cyclopentyl (C₅), n-hexyl (C₆), and cyclohexyl (C₆);
ii) —(CR^{35a}R^{35b})_{q}OR³⁴; for example, —OH, —CH₂OH, —OCH₃, —CH₂OCH₃, —OCH₂CH₃, —CH₂OCH₂CH₃, —OCH₂CH₂CH₃, and —CH₂OCH₂CH₂CH₃;
iii) —(CR^{35a}R^{35b})_{q}C(O)R³⁴; for example, —COCH₃, —CH₂COCH₃, —OCH₂CH₃,
   —CH₂COCH₂CH₃, —COCH₂CH₂CH₃, and —CH₂COCH₂CH₂CH₃;
iv) —(CR^{35a}R^{35b})_{q}C(O)OR³⁴; for example, —CO₂CH₃, —CH₂CO₂CH₃, —CO₂CH₂CH₃, —CH₂CO₂CH₂CH₃, —CO₂CH₂CH₂CH₃, and —CH₂CO₂CH₂CH₂CH₃;
v) —(CR^{35a}R^{35b})_{q}C(O)N(R³⁴)₂; for example, —CONH₂, —CH₂CONH₂, —CONHCH₃, —CH₂CONHCH₃, —CON(CH₃)₂, and —CH₂CON(CH₃)₂;
vi) —(CR^{3a}R^{35b})_{q}OC(O)N(R³⁴)₂; for example, —OC(O)NH₂, —CH₂OC(O)NH₂, —OC(O)NHCH₃, —CH₂OC(O)NHCH₃, —OC(O)N(CH₃)₂, and —CH₂OC(O)N(CH₃)₂;
vii) —(CR^{35a}R^{35b})_{q}N(R³⁴)₂; for example, —NH₂, —CH₂NH₂, —NHCH₃, —N(CH₃)₂, —NH(CH₂CH₃), —CH₂NHCH₃, —CH₂N(CH₃)₂, and —CH₂NH(CH₂CH₃); viii) halogen: —F, —Cl, —Br, and —I;
ix) —CHₘXₙ; wherein X is halogen, m is from 0 to 2, m+n=3; for example, —CH₂F, —CHF₂, —CF₃, —CCl₃, or —CBr₃;
x) —(CR^{35a}R^{35b})_{q}CN; for example; —CN, —CH₂CN, and —CH₂CH₂CN;
xi) —(CR^{35a}R^{35b})_{q}NO₂; for example; —NO₂, —CH₂NO₂, and —CH₂CH₂NO₂;
xii) —(CR^{35a}R^{35b})_{q}SO₂R³⁴; for example, —SO₂H, —CH₂SO₂H, —SO₂CH₃, —CH₂SO₂CH₃, —SO₂C₆H₅, and —CH₂SO₂C₆H₅; and
xiii) —(CR^{35a}R^{35b})_{q}SO₃R³⁴; for example, —SO₃H, —CH₂SO₃H, —SO₃CH₃, —CH₂SO₃CH₃, —SO₃C₆H₅, and —CH₂SO₃C₆H₅;
   wherein each R³⁴ is independently hydrogen, substituted or unsubstituted C₁-C₄ linear, branched, or cyclic alkyl; or two R³⁴ units can be taken together to form a ring comprising 3-7 atoms; R^{35a} and R^{35b} are each independently hydrogen or C₁-C₄ linear or branched alkyl; the index p is from 0 to 4.

Compound according to the first embodiment of this category can be prepared by the procedure outlined herein below in Scheme IV which is a modified procedure as described in U.S. Pat. No. 4,785,008 included herein by reference in its entirety.

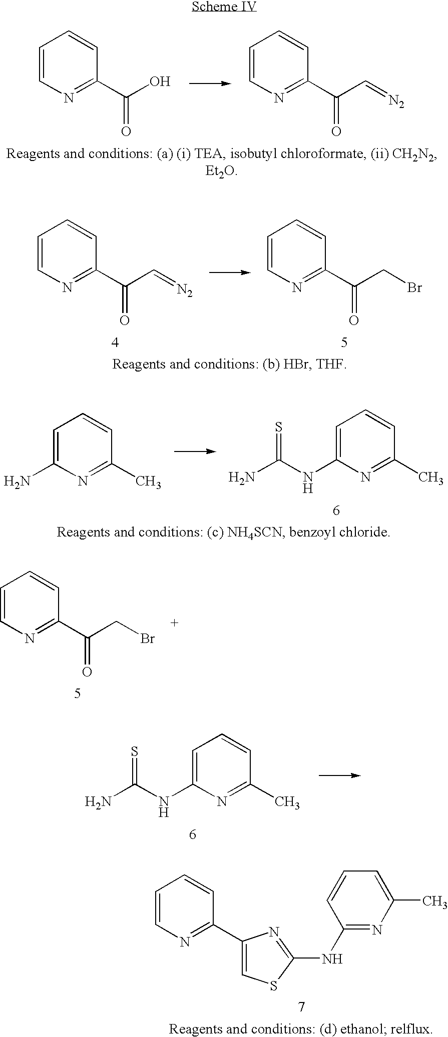

Preparation of 2-diazo-1-pyridin-2-yl)ethanone (4): To a 0° C. solution of picolinic acid (492 mg, 4.0 mmol) in THF (20 mL) is added dropwise triethylamine (0.61 mL, 4.4 mmol) followed by iso-butyl chloroformate (0.57 mL, 4.4 mmol). The reaction mixture is stirred at 0° C. for 20 minutes and filtered. The filtrate is treated with an ether solution of diazomethane (˜16 mmol) at 0° C. The reaction mixture is stirred at room temperature for 3 hours then concentrated in vacuo. The resulting residue is dissolved in EtOAc and washed successively with water and brine, dried (Na₂SO₄), filtered and concentrated. The residue can be purified over silica to afford the desired product.

Preparation of 2-bromo-1-(pyridin-2-yl)ethanone (5): To a 0° C. solution of 2-diazo-1-pyridin-2-yl)ethanone, 4, (153 mg, 1.04 mmol) in THF (5 mL) is added dropwise 48% aq. HBr (0.14 mL, 1.25 mmol). The reaction mixture is stirred at 0° C. for 1.5 hours then the reaction is quenched at 0° C. with sat. Na₂CO₃. The mixture is extracted with EtOAc (3×25 mL) and the combined organic extracts are washed with brine, dried (Na₂SO₄), filtered and concentrated to obtain the desire product that can be used in the next step without further purification.

Preparation of 1-(6-methylpyridin-2-yl)thiourea (6): Benzoyl chloride (200 mmol) is added dropwise to a solution of ammonium thiocyanate (220 mmol) in anhydrous acetone (100 mL). The mixture is heated to reflux for about 5 minutes once the addition is complete. A solution of 2-amino-6-methylpyridine (21.6 g, 200 mmol) in anhydrous acetone (50 mL) is added dropwise at a rate that maintains a gentle reflux. The solution is stirred an additional 5 minutes then poured into ice-cold water (1.5 L). The crystals that form are collected by filtration and suspended in 10% NaOH (300 mL). The suspension is boiled for approximately 5 minutes then conc. HCl is added. The solution is then adjusted to pH 8 with ammonium hydroxide. The product obtained can be purified or used as is for the next step.

Preparation of N-(6-methylpyridin-2-yl)-4-(pyridine-2-yl)thiazol-2-amine (7): 1-(6-methylpyridin-2-yl)thiourea, 6, (10.9 g, 65 mmol) and 2-bromo-1-(pyridin-2-yl)ethanone, 5, (15.4 g, 77 mmol) in ethanol (100 mL) are brought to reflux for 3 hours. The solvent is removed in vacuo to afford the desire product.

Another embodiment of this category relates to compounds having the formula:

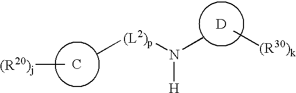

wherein C represents a substituted or unsubstituted phenyl or a substituted or unsubstituted heteroaryl ring having from 6 to 10 atoms. D represents a substituted or unsubstituted heteroaryl ring having from 6 to 10 atoms. R²⁰, R³⁰, L² and the indices j, k, and p are the same as defined herein above.

C is a substituted or unsubstituted phenyl or a substituted or unsubstituted heteroaryl ring having from 6 to 10 atoms and D represents a substituted or unsubstituted heteroaryl ring having from 6 to 10 atoms. Non-limiting examples of heteroaryl rings according to this embodiment include:

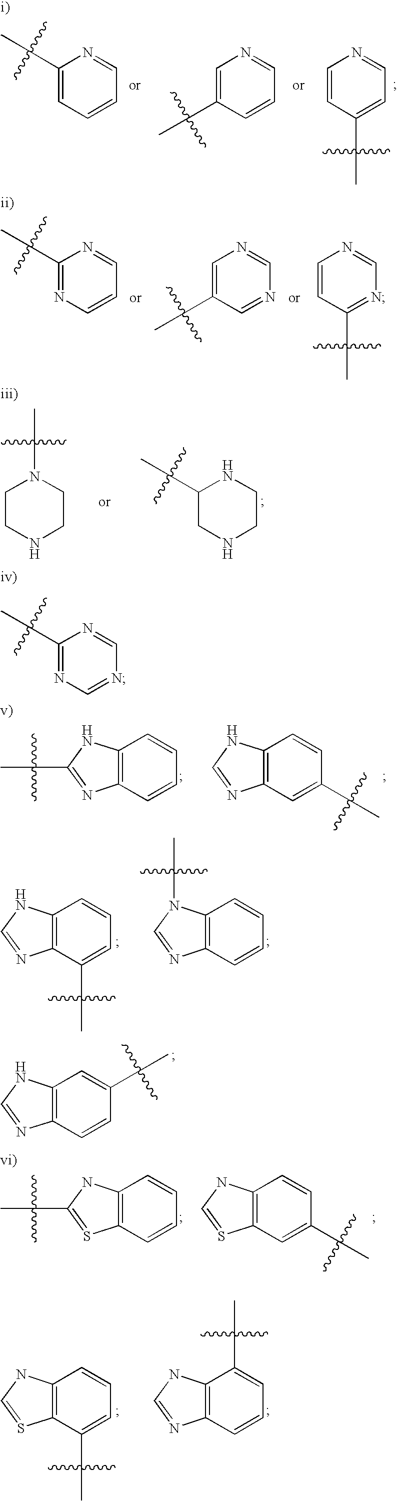

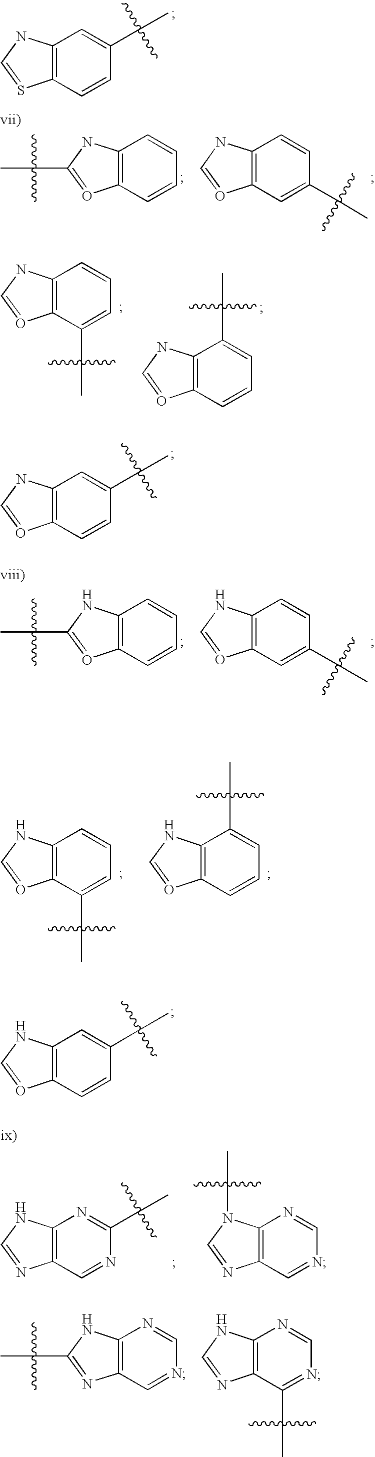

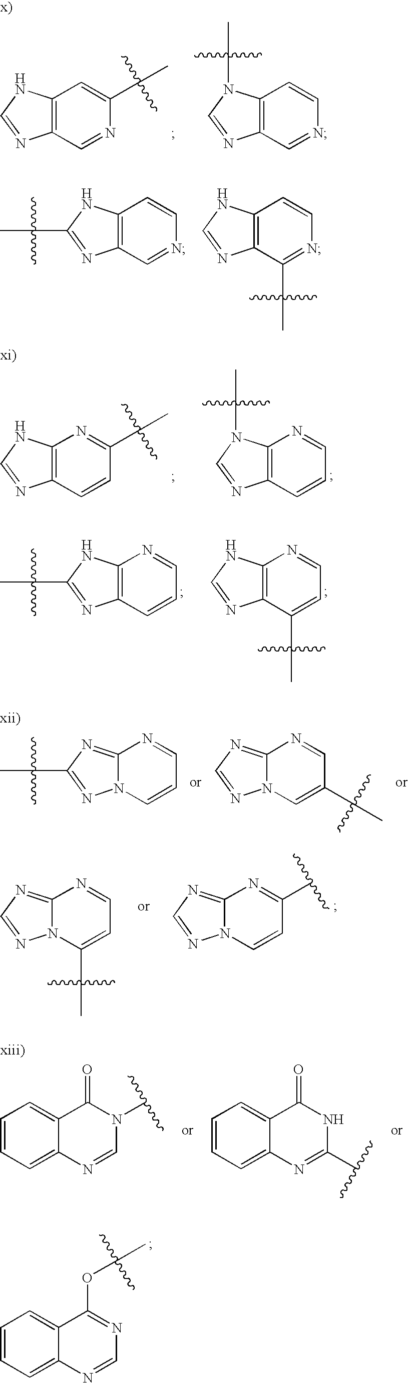

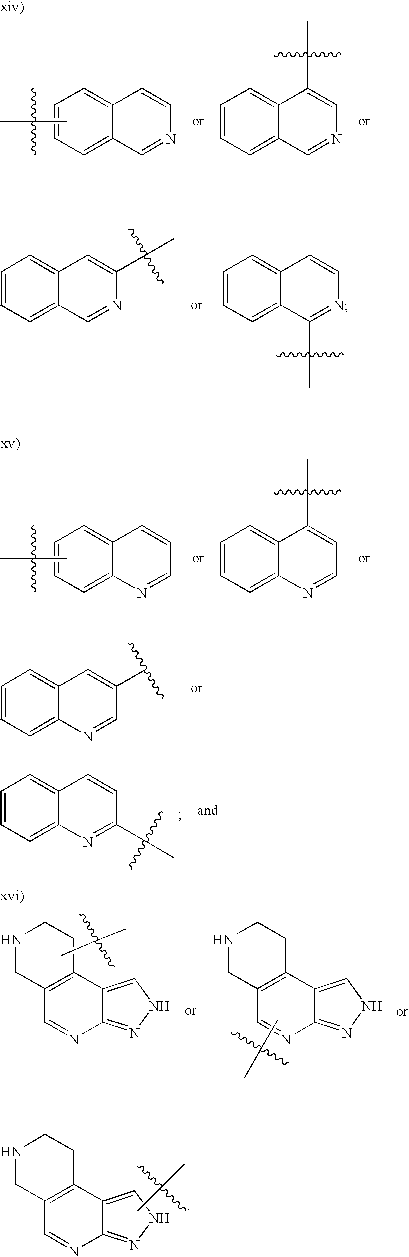

The following Table 2 provides examples of tissue-nonspecific alkaline phosphatase (TNAP) activators.

TABLE 2
TNAP activators

| | TNAP activation |
|---|---|
| Compound | factor |
| 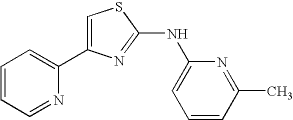 | 6.1 |
| N-(6-methylpyridin-2-yl)-4-(pyridine-2-yl)thiazol-2- | |
| amine | |
| 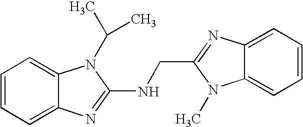 | 2.0 |
| 1-isopropyl-N-[(1-methyl-1H-benzo[d]imidazol-2- | |
| yl)methyl]-1H-benzo[d]imidazol-2-amine | |
| 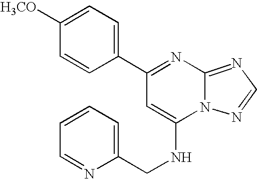 | 2.0 |
| 5-(4-methoxyphenyl)-N-(pyridine-2-ylmethyl)- | |
| [1,2,4]triazole[1,5-a]pyrimidin-7-amine | |
| 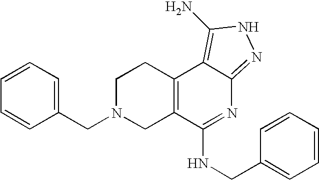 | 1.9 |
| N⁵,7-dibenzyl-6,7,8,9-tetrahydro-2H-pyrazolo[3,4- | |
| c][2,7]napythyridine-1,5-diamine | |

The third category of tissue-nonspecific alkaline phosphatase activators of the present disclosure are substituted heteroaryl rings comprising from 5 to 11 atoms, wherein the heteroatom can be one or more nitrogen, oxygen, or sulfur atoms. The heteroaryl rings can be substituted by one or more organic radicals independently chosen from:
i) linear, branched, or cyclic alkyl, alkenyl, and alkynyl; for example, methyl (C₁), ethyl (C₂), n-propyl (C₃), iso-propyl (C₃), cyclopropyl (C₃), propylen-2-yl (C₃), propargyl (C₃), n-butyl (C₄), iso-butyl (C₄), sec-butyl (C₄), tert-butyl (C₄), cyclobutyl (C₄), n-pentyl (C₅), cyclopentyl (C₅), n-hexyl (C₆), and cyclohexyl (C₆);
ii) substituted or unsubstituted aryl attached to the heteroaryl ring by a polyalkylene tether having from 1 to 6 carbon atoms in the chain; for example, phenyl, benzyl, 2-phenylethyl, 2-fluorophenyl, 3-chlorophenyl, 4-methylphenyl, 2-aminophenyl, 3-hydroxyphenyl, 4-trifluoromethyl-phenyl, and biphenyl-4-yl;
iii) substituted or unsubstituted heterocyclic attached to the heteroaryl ring by a polyalkylene tether having from 1 to 6 carbon atoms in the chain; for example, piperidinyl, piperidin-1-ylmethyl; pyrrolidinyl, and morpholinyl;
iv) substituted or unsubstituted heteroaryl attached to the heteroaryl ring by a polyalkylene tether having from 1 to 6 carbon atoms in the chain; for example, pyrrolyl, pyridinyl, pyridine-2-ylmethyl, pyrimidinyl, and pyrimidin-4-ylmethyl;
v) —(CR^{43a}R^{43b})_{q}OR⁴²; for example, —OH, —CH₂OH, —OCH₃, —CH₂OCH₃, —OCH₂CH₃, —CH₂OCH₂CH₃, —OCH₂CH₂CH₃, and —CH₂OCH₂CH₂CH₃;
vi) —(CR^{43a}R^{43b})_{q}C(O)R⁴²; for example, —COCH₃, —CH₂COCH₃, —OCH₂CH₃, —CH₂COCH₂CH₃, —COCH₂CH₂CH₃, and —CH₂COCH₂CH₂CH₃;
vii) —(CR^{43a}R^{43b})_{q}C(O)OR⁴²; for example, —CO₂CH₃, —CH₂CO₂CH₃, —CO₂CH₂CH₃, —CH₂CO₂CH₂CH₃, —CO₂CH₂CH₂CH₃, and —CH₂CO₂CH₂CH₂CH₃;
viii) —(CR^{43a}R^{43b})_{q}C(O)N(R⁴²)₂; for example, —CONH₂, —CH₂CONH₂, —CONHCH₃, —CH₂CONHCH₃, —CON(CH₃)₂, and —CH₂CON(CH₃)₂;
ix) —(CR^{43a}R^{43b})_{q}OC(O)N(R⁴²)₂; for example, —OC(O)NH₂, —CH₂OC(O)NH₂, —OC(O)NHCH₃, —CH₂OC(O)NHCH₃, —OC(O)N(CH₃)₂, and —CH₂OC(O)N(CH₃)₂;
x) —(CR^{43a}R^{43b})_{q}N(R⁴²)₂; for example, —NH₂, —CH₂NH₂, —NHCH₃, —N(CH₃)₂, —NH(CH₂CH₃), —CH₂NHCH₃, —CH₂N(CH₃)₂, and —CH₂NH(CH₂CH₃);
xi) halogen: —F, —Cl, —Br, and —I;
xii) —CHₘXₙ; wherein X is halogen, m is from 0 to 2, m+n=3; for example, —CH₂F, —CHF₂, —CF₃, —CCl₃, or —CBr₃;
xiii) —(CR^{43a}R^{43b})_{q}CN; for example; —CN, —CH₂CN, and —CH₂CH₂CN;
xiv) —(CR^{43a}R^{43b})_{q}NO₂; for example; —NO₂, —CH₂NO₂, and —CH₂CH₂NO₂;
xv) —(CR^{43a}R^{43b})_{q}SO₂R⁴²; for example, —SO₂H, —CH₂SO₂H, —SO₂CH₃, —CH₂SO₂CH₃, —SO₂C₆H₅, and —CH₂SO₂C₆H₅; and
xvi) —(CR^{43a}R^{43b})_{q}SO₃R⁴²; for example, —SO₃H, —CH₂SO₃H, —SO₃CH₃, —CH₂SO₃CH₃, —SO₃C₆H₅, and —CH₂SO₃C₆H₅;
   wherein each R² is independently hydrogen, substituted or unsubstituted C₁-C₄ linear, branched, or cyclic alkyl; or two R² units can be taken together to form a ring comprising 3-7 atoms; R^{43a} and R^{43b} are each independently hydrogen or C₁-C₄ linear or branched alkyl; the index q is from 0 to 4.

When the organic radical that substitutes for a hydrogen atom of the heteroaryl rings of this category comprises C₁-C₁₂ linear, branched, or cyclic alkyl, alkenyl; substituted or unsubstituted C₆ or C₁₀ aryl; substituted or unsubstituted C₁-C₉heterocyclic; or substituted or unsubstituted C₁-C₉heteroaryl; the organic radical can further have one or more hydrogen atoms substituted by one or more organic radicals. Non-limiting examples of organic radicals that can substitute for a hydrogen atom include:
i) linear, branched, or cyclic alkyl, alkenyl, and alkynyl; for example, methyl (C₁), ethyl (C₂), n-propyl (C₃), iso-propyl (C₃), cyclopropyl (C₃), propylene-2-yl (C₃), propargyl (C₃), n-butyl (C₄), iso-butyl (C₄), sec-butyl (C₄), tert-butyl (C₄), cyclobutyl (C₄), n-pentyl (C₅), cyclopentyl (C₅), n-hexyl (C₆), and cyclohexyl (C₆);
ii) —(CR^{45a}R^{45b})_{q}OR^{4r}; for example, —OH, —CH₂OH, —OCH₃, —CH₂OCH₃, OCH₂CH₃, —CH₂OCH₂CH₃, —OCH₂CH₂CH₃, and —CH₂OCH₂CH₂CH₃;
iii)-(CR^{45a}R^{45b})_{q}C(O)R^{4r}; for example, —COCH₃, —CH₂COCH₃, —OCH₂CH₃, —CH₂COCH₂CH₃, —COCH₂CH₂CH₃, and —CH₂COCH₂CH₂CH₃;
iv) —(CR^{45a}R^{45b})_{q}C(O)OR^{4r}; for example, —CO₂CH₃, —CH₂CO₂CH₃, —CO₂CH₂CH₃, —CH₂CO₂CH₂CH₃, —CO₂CH₂CH₂CH₃, and —CH₂CO₂CH₂CH₂CH₃;
v) —(CR^{45a}R^{45b})_{q}C(O)N(R^{4r})₂; for example, —CONH₂, —CH₂CONH₂, —CONHCH₃, —CH₂CONHCH₃, —CON(CH₃)₂, and —CH₂CON(CH₃)₂;
vi) —(CR^{45a}R^{45b})_{q}OC(O)N(R^{4r})₂; for example, —OC(O)NH₂, —CH₂OC(O)NH₂, —OC(O)NHCH₃, —CH₂OC(O)NHCH₃, —OC(O)N(CH₃)₂, and —CH₂OC(O)N(CH₃)₂;
vii) —(CR^{45a}R^{45b})_{q}N(R^{4r})₂; for example, —NH₂, —CH₂NH₂, —NHCH₃, —N(CH₃)₂, —NH(CH₂CH₃), —CH₂NHCH₃, —CH₂N(CH₃)₂, and —CH₂NH(CH₂CH₃);
viii) halogen: —F, —Cl, —Br, and —I;
ix) —CHₘXₙ; wherein X is halogen, m is from 0 to 2, m+n=3; for example, —CH₂F, —CHF₂, —CF₃, —CCl₃, or —CBr₃;
x) —(CR^{45a}R^{45b})_{q}CN; for example; —CN, —CH₂CN, and —CH₂CH₂CN;
xi) —(CR^{45a}R^{45b})_{q}NO₂; for example; —NO₂, —CH₂NO₂, and —CH₂CH₂NO₂;
xii) —(CR^{45a}R^{45b})_{q}SO₂R^{4r}; for example, —SO₂H, —CH₂SO₂H, —SO₂CH₃, —CH₂SO₂CH₃, —SO₂C₆H₅, and —CH₂SO₂C₆H₅; and
xiii) —(CR^{45a}R^{45b})_{q}SO₃R^{4r}; for example, —SO₃H, —CH₂SO₃H, —SO₃CH₃, —CH₂SO₃CH₃, —SO₃C₆H₅, and —CH₂SO₃C₆H₅;
   wherein each R^{4r} is independently hydrogen, substituted or unsubstituted C₁-C₄ linear, branched, or cyclic alkyl; or two R^{4r} units can be taken together to form a ring comprising 3-7 atoms; R^{45a} and R^{45b} are each independently hydrogen or C₁-C₄ linear or branched alkyl; the index p is from 0 to 4.

A first embodiment includes heteroaryl rings comprising 6 carbon atoms and 3 nitrogen atoms, for example, a substituted 7H-pyrrolo[2,3-d]pyrimidine having the formula:

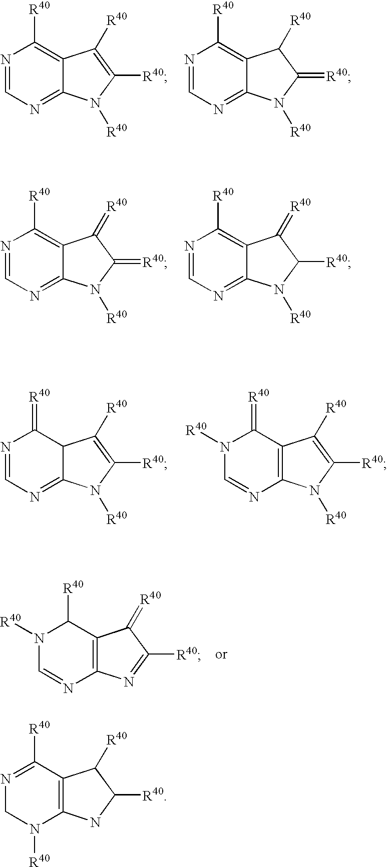

Non-limiting examples of this embodiment include:
i) 3-[3-(1H-imidazol-1-yl)propyl]-7-benzyl-5,6-diphenyl-3H-pyrrolo[2,3-d]pyrimidin-4(7H)-imine:

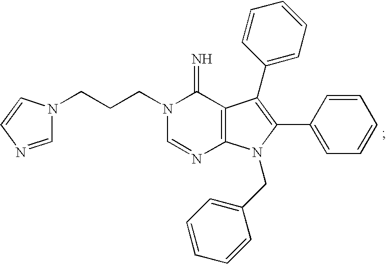

ii) 7-(diethylamino)-3-(1-methyl-1H-benzo[d]imidazol-2-yl)-2H-chromen-2-one:

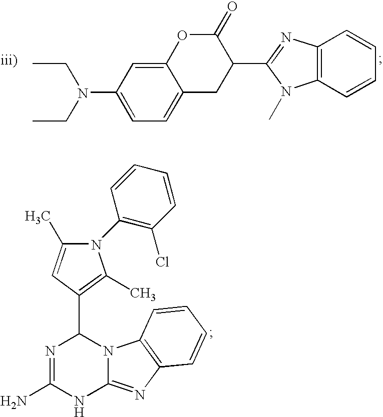

iv) 5-tert-butyl-2-methyl-3-phenylpyrazolo[1,5-a]pyrimidin-7-ol:

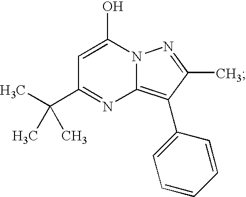

v) 7-[morpholino(pyridin-2-yl)methyl]quinolin-8-ol:

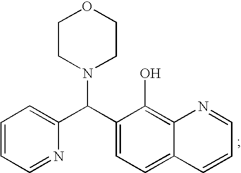

vi) 2,2′,2″,2′″-[4,8-di(piperidin-1-yl)pyrimido[5,4-d]pyrimidine-2,6-diyl]bis(azanetriyl)tetraethanol;

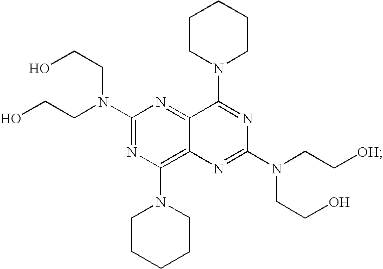

vii) 3-(3-phenylpyridazino[3,4-b]quinoxalin-5(10H)-yl)propan-1-ol:

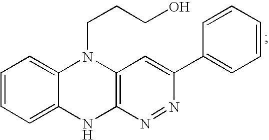

viii) 6-cyclohexyl-3-(2,4,5,6-tetrahydrocyclopenta[c]pyrazol-3-yl)-[1,2,4]triazole[3,4-b][1,3,4]thiadiazole:
ix)

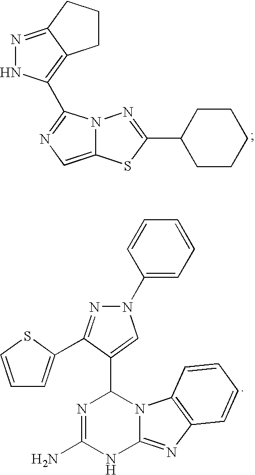

The following are compounds that can be used as tissue-nonspecific alkaline phosphatase activators:
i) 5,5,7,12,12,14-hexamethyl-1,4,8,11-tertraazacyclotetradecane:

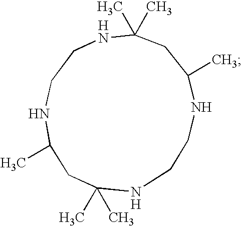

ii) 2,2′,2″-(1-oxa-4,7,10-triazacyclododecane-4,7,10-triyl)ethanol

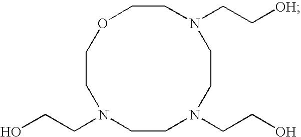

iii) N-(3,4-dimethoxyphenethyl)-5-(2-hydroxyphenyl)-1H-pyrazole-3-carboxamide

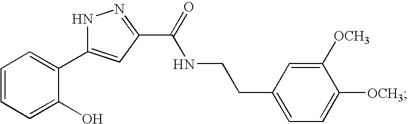

iv) N-[2-(4-fluorobenzylamino)-2-oxoethyl]-2-(4-fluorphenylsulfonamido)-N-(furan-2-ylmethyl)acetamide

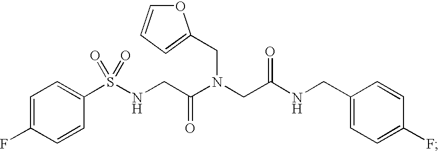

v) 5-bromo-N-[3-(trifluoromethoxy)phenyl]furan-2-carboxamide
vi)

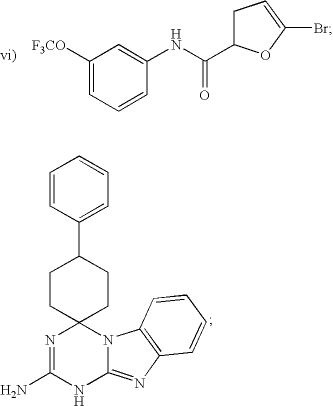

vii) 2-[2-(naphthalene-2-ylsulfonyl)ethyl]-5-phenyl-1,3,4-oxadiazole

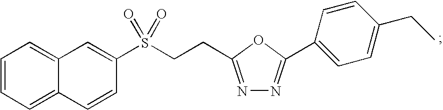

viii) N-{2-[ethyl(phenyl)amino]ethyl}-1-{[2-(4-ethylphenyl)-5-methyloxazol-4-yl]methyl}piperidine-4-carboxamide

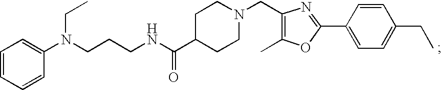

ix) N-[1-(2,6-dimethylphenylcarbamoyl)cyclohexyl]-N-(3-methoxyphenyl)-1H-pyrazole-3-carboxamide

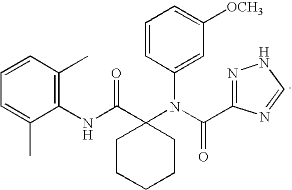

The following Table 3 provides examples of the disclosed tissue-nonspecific alkaline phosphatase (TNAP) activators according to the present disclosure.

TABLE 3
TNAP activators

| | TNAP activation |
|---|---|
| Compound | factor |
| 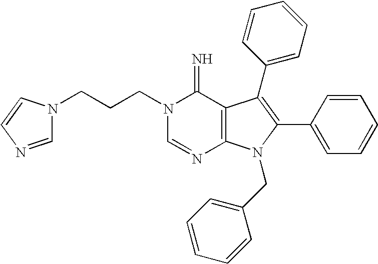 | 4.3 |
| 3-[3-(1H-imidazol-1-yl)propyl]-7-benzyl-5,6-diphenyl-3H- | |
| pyrrolo[2,3-d]pyrimidin-4(7H)-imine | |
| 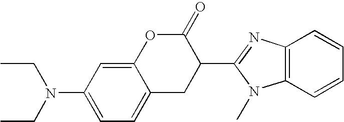 | 2.8 |
| 7-(diethylamino)-3-(1-methyl-1H-benzo[d]imidazol-2-yl)-2H- | |
| chromen-2-one | |
| 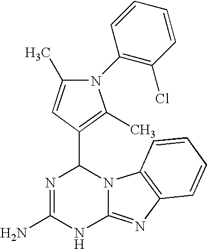 | 2.2 |
| 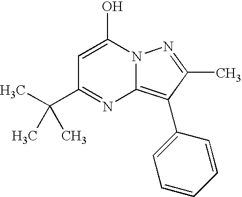 | 1.8 |
| 5-tert-butyl-2-methyl-3-phenylpyrazolo[1,5-a]pyrimidin-7-ol | |
| 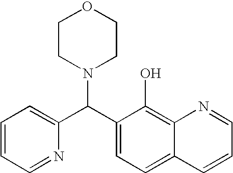 | 1.7 |
| 7-[morpholino(pyridin-2-yl)methyl]quinolin-8-ol | |
| 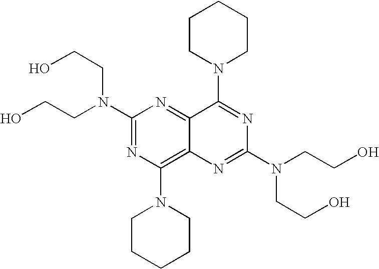 | 1.6 |
| 2,2′,2″,2′″-[4,8-di(piperidin-1-yl)pyrimido[5,4-d]pyrimidine-2,6- | |
| diyl]bis(azanetriyl)tetraethanol | |
| 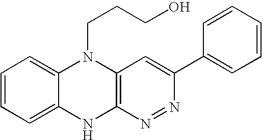 | 1.5 |
| 3-(3-phenylpyridazino[3,4-b]quinoxalin-5(10H)-yl)propan-1-ol | |
| 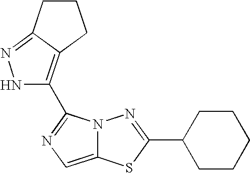 | 1.5 |
| 6-cyclohexyl-3-(2,4,5,6-tetrahydrocyclopenta[c]pyrazol-3-yl)- | |
| [1,2,4]triazole[3,4-b][1,3,4]thiadiazole | |
| 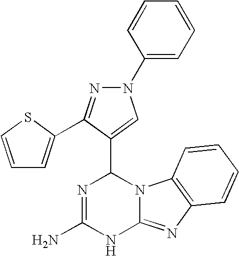 | 1.5 |
| 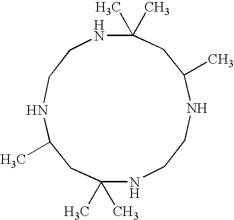 | 2.3 |
| 5,5,7,12,12,14-hexamethyl-1,4,,8,11-tertraazacyclotetradecane | |
| 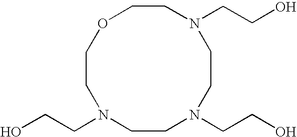 | 1.9 |
| 2,2′,2″-(1-oxa-4,7,10-triazacyclododecane-4,7,10-triyl)ethanol | |
| 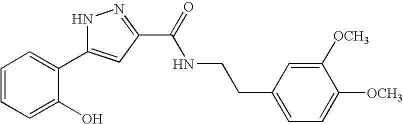 | 1.9 |
| N-(3,4-dimethoxyphenethyl)-5-(2-hydroxyphenyl)-1H-pyrazole-3- | |
| carboxamide | |
| 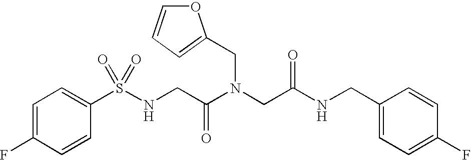 | 1.7 |
| N-[2-(4-fluorobenzylamino)-2-oxoethyl]-2-(4- | |
| fluorphenylsulfonamido)-N-(furan-2-ylmethyl)acetamide | |
| 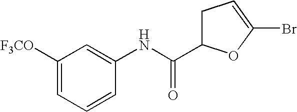 | 1.6 |
| 5-bromo-N-[3-(trifluoromethoxy)phenyl]furan-2-carboxamide | |
| 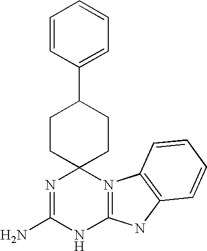 | 1.6 |
| 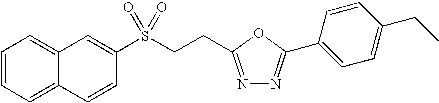 | 1.6 |
| 2-[2-(naphthalene-2-ylsulfonyl)ethyl]-5-phenyl-1,3,4-oxadiazole | |
| 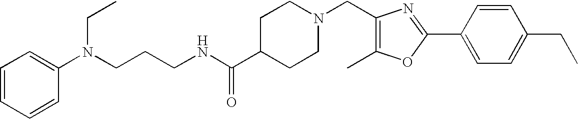 | 1.6 |
| N-{2-[ethyl(phenyl)amino]ethyl}-1-{[2-(4-ethylphenyl)-5- | |
| methyloxazol-4-yl]methyl}piperidine-4-carboxamide | |
| 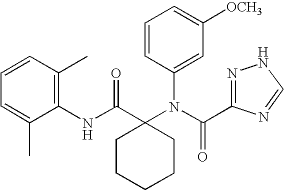 | 1.6 |
| N-[1-(2,6-dimethylphenylcarbamoyl)cyclohexyl[-N-(3- | |
| methoxyphenyl)-1H-pyrazole-3-carboxamide | |

2. Formulations

The present disclosure also relates to compositions or formulations which comprise the tissue non-specific alkaline phosphatase activators according to the present disclosure. In general, the compositions of the present disclosure comprise:
a) an effective amount of one or more tissue non-specific alkaline phosphatase activators according to the present disclosure can be used for hypophosphatasia, osteoporosis, or calcium pyrophosphate deposition disease (CPPD/chodrocalcinosis); and
b) one or more excipients.

For example, disclosed herein is a formulation comprising an effective amount of tissue non-specific alkaline phosphatase used to manipulate extracellular inorganic phosphate-to-pyrophosphate ratio in an animal. In some aspects, the manipulation is achieved by increasing the degradation of pyrophosphatase. In these aspects, the degradation of pyrophosphatase is typically increased by activating tissue non-specific alkaline phosphatase's pyrophosphatase activity. The formulation can be used to treat an individual is suffering from a disease selected from the group consisting of perinatal hypophosphatasia, infantile hypophosphatasia, childhood hypophosphatasia, adult hypophosphatasia, odontohypophosphatasia, pseudohypophosphatasia and osteoporosis. The formulation can further comprise a pharmaceutically acceptable carrier as described below.

The formulator will understand that excipients are used primarily to serve in delivering a safe, stable, and functional pharmaceutical, serving not only as part of the overall vehicle for delivery but also as a means for achieving effective absorption by the recipient of the active ingredient. An excipient may fill a role as simple and direct as being an inert filler, or an excipient as used herein may be part of a pH stabilizing system or coating to insure delivery of the ingredients safely to the stomach. The formulator can also take advantage of the fact the compounds of the present disclosure have improved cellular potency, pharmacokinetic properties, as well as improved oral bioavailability.

Non-limiting examples of compositions according to the present disclosure include:
a) from about 0.001 mg to about 1000 mg of one or more tissue non-specific alkaline phosphatase activators according to the present disclosure; and
b) one or more excipients.

Another example according to the present disclosure relates to the following compositions:
a) from about 0.01 mg to about 100 mg of one or more tissue non-specific alkaline phosphatase activators according to the present disclosure; and
b) one or more excipients.

A further example according to the present disclosure relates to the following compositions:
a) from about 0.1 mg to about 10 mg of one or more human protein tissue non-specific alkaline phosphatase activators according to the present disclosure; and
b) one or more excipients.

The term “effective amount” as used herein means “an amount of one or more tissue non-specific alkaline phosphatase activators, effective at dosages and for periods of time necessary to achieve the desired or therapeutic result.” An effective amount may vary according to factors known in the art, such as the disease state, age, sex, and weight of the human or animal being treated. Although particular dosage regimes may be described in examples herein, a person skilled in the art would appreciated that the dosage regime may be altered to provide optimum therapeutic response. For example, several divided doses may be administered daily or the dose may be proportionally reduced as indicated by the exigencies of the therapeutic situation. In addition, the compositions of the present disclosure can be administered as frequently as necessary to achieve a therapeutic amount.

As described herein above, the formulations of the present disclosure include pharmaceutical compositions comprising a compound that can inhibit the activity of HePTP and therefore is suitable for use in hypophosphatasia, osteoporosis, or calcium pyrophosphate deposition disease (CPPD/chodrocalcinosis) (or a pharmaceutically-acceptable salt thereof) and a pharmaceutically-acceptable carrier, vehicle, or diluent. Those skilled in the art based upon the present description and the nature of any given activator identified by the assays of the present invention will understand how to determine a therapeutically effective dose thereof.

The pharmaceutical compositions may be manufactured using any suitable means, e.g., by means of conventional mixing, dissolving, granulating, dragee-making, levigating, emulsifying, encapsulating, entrapping or lyophilizing processes.

Pharmaceutical compositions for use in accordance with the present disclosure thus may be formulated in a conventional manner using one or more physiologically or pharmaceutically acceptable carriers (vehicles, or diluents) comprising excipients and auxiliaries which facilitate processing of the active compounds into preparations which can be used pharmaceutically. Proper formulation is dependent upon the route of administration chosen.

Any suitable method of administering a pharmaceutical composition to a patient may be used in the methods of treatment of the present invention, including injection, transmucosal, oral, inhalation, ocular, rectal, long acting implantation, liposomes, emulsion, or sustained release means.

For injection, the agents of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art. For ocular administration, suspensions in an appropriate saline solution are used as is well known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained as a solid excipient, optionally grinding a resulting mixture, and processing the mixture of granules, after adding suitable auxiliaries, if desired, to obtain tablets or dragee cores. Suitable excipients include fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinyl-pyrrolidone (PVP). If desired, disintegrating agents may be added, such as cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin, for use in an inhaler or insufflator, may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, such as sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

One type of pharmaceutical carrier for hydrophobic compounds of the invention is a cosolvent system comprising benzyl alcohol, a nonpolar surfactant, a water-miscible organic polymer, and an aqueous phase.

The cosolvent system may be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g., polyvinyl pyrrolidone; and other sugars or polysaccharides may be substituted for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed.

Additionally, the compounds may be delivered using any suitable sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a prolonged period of time. Depending on the chemical nature and the biological stability of the therapeutic reagent, additional strategies for protein stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Many of the agents of the invention may be provided as salts with pharmaceutically acceptable counterions. Salts tend to be more soluble in aqueous or other protonic solvents than are the corresponding free base forms.

### B. Methods

As disclosed herein, removal of PPᵢ via TNAP action and the presence of a fibrilar collagen-rich scaffold are two conditions necessary to induce mineralization of bone or any ECM. Further as disclosed herein, the Pᵢ/PPᵢ ratio is of fundamental significance for bone ECM mineralization. Thus, in the bone ECM, while the extracellular Pᵢ concentration is fairly constant, TNAP's enzymatic degradation of PPᵢ controls the Pᵢ/PPᵢ ratio to favor crystallization of hydroxyapatite (HA) outside the MVs along collagen fibrils. This axis was tested by surmising that transgenic mice over-expressing TNAP can achieve tissular expression of TNAP sufficiently high to be able to lower circulating PPᵢ concentrations to enhance bone mineral density (BMD) in these animals. Transgenic mice were generated by expressing human TNAP cDNA under control of the Apolipoprotein E promoter, which drives expression of TNAP primarily in the post-natal liver. The expression levels of TNAP was examined in tissues from mice carrying one copy or two copies of the ApoE-Tnap transgene and also from [Akp2^{-/-}; ApoE-Tnap] mice, and examined the ability of their primary osteoblasts to calcify in culture. MicroCT (μCT) analysis was used to measure BMD in long bones, vertebrae and calvaria. TNAP expression in ApoE-Tnap mice was major in the liver and kidney as expected, with lower but yet detectable levels in bone, brain and lung. Serum AP concentrations were 10 to 50-fold higher than age-matched sibling control wild-type (WT) mice. As predicted, serum levels of PPᵢ were reduced in the transgenic animals. Furthermore, μCT analysis of femur, vertebrae and calvaria revealed higher BMD in cancellous bone of ApoE-Tnap⁺ and ApoE-Tnap^{+/+} mice compared to WT mice. Thus, increases in tissular and circulating levels of TNAP lead to higher BMD by reducing the effective levels of the calcification inhibitor PPᵢ. Further, administration of recombinant TNAP itself, or of pharmacological activators of TNAP's pyrophosphatase activity, can serve as therapeutics drugs for the treatment of osteoporosis.

1. Methods of Treatment

Provided herein is a method of promoting bone mineral deposition in a subject, comprising administering to the subject a tissue-nonspecific alkaline phosphatase (TNAP) activator. Also provided is a method of increasing bone mineral density (BMD) in a subject, comprising administering to the subject in need thereof a TNAP activator.

Also provided is a method of treating a heritable skeletal disease by administering an amount of TNAP activator sufficient to lower circulating pyrophosphate concentrations. The heritable skeletal disease can be osteoporosis or hypophosphatasia. The amount of TNAP activator can be sufficient to lower circulating osteopontin concentrations. The amount of TNAP activator can be sufficient to enhance bone mineral density in an animal.

Also provided is a method of improving long term survival and skeletal mineralization in an individual with symptoms of hypophosphatasia comprising administration of enzyme replacement therapy, wherein the enzyme replacement therapy includes administration of tissue non-specific alkaline phosphatase and further comprising administering a TNAP activator.

In some aspects of the disclosed methods, the subject has been diagnosed with hypophosphatasia. In some aspects of the disclosed methods, the subject has been diagnosed with osteoporosis. In some aspects of the disclosed methods, the subject has been diagnosed with calcium pyrophosphate deposition disease (CPPD/chodrocalcinosis).

Thus, also provided herein is a method of treating hypophosphatasia in a subject, comprising administering to the subject in need thereof a TNAP activator. Thus, also provided is a method of treating osteoporosis in a subject, comprising administering to the subject in need thereof a TNAP activator. Thus, also provided is a method of treating calcium pyrophosphate deposition disease (CPPD/chodrocalcinosis) in a subject, comprising administering to the subject in need thereof a TNAP activator.

Any of the herein provided methods can further comprise administering to the subject a TNAP peptide.

Also provided is a method of enhancing the pyrophosphatase activity of tissue-nonspecific alkaline phosphatase (TNAP), comprising contacting the TNAP with a TNAP activator. Although not wishing to be bound by theory, the disclosed TNAP activator can facilitate the release of inorganic pyrophosphate (PPᵢ) from the active site, thereby increasing the effective rate of PPᵢ hydrolysis.

The TNAP activator of the provided methods can be a macromolecule, such as a polymer. The TNAP activator of the provided methods can be a small molecule. Thus, the TNAP activator can be a compound disclosed herein. The TNAP activator can further be a compound identified as disclosed herein.

2. Administration

The disclosed compounds and compositions can be administered in any suitable manner. The manner of administration can be chosen based on, for example, whether local or systemic treatment is desired, and on the area to be treated. For example, the compositions can be administered orally, parenterally (e.g., intravenous, subcutaneous, intraperitoneal, or intramuscular injection), by inhalation, extracorporeally, topically (including transdermally, ophthalmically, vaginally, rectally, intranasally) or the like.

As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The exact amount of the compositions required can vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein. Thus, effective dosages and schedules for administering the compositions may be determined empirically, and making such determinations is within the skill in the art. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage can vary with the age, condition, sex and extent of the disease in the patient, route of administration, or whether other drugs are included in the regimen, and can be determined by one of skill in the art. The dosage can be adjusted by the individual physician in the event of any counter indications. Dosage can vary, and can be administered in one or more dose administrations daily, for one or several days. Guidance can be found in the literature for appropriate dosages for given classes of pharmaceutical products.

For example, a typical daily dosage of a TNAP activator disclosed herein used alone might range from about 1 μg/kg to up to 100 mg/kg of body weight or more per day, depending on the factors mentioned above.

Following administration of a disclosed composition for promoting bone mineral deposition, the efficacy of the therapeutic composition can be assessed in various ways well known to the skilled practitioner. For instance, one of ordinary skill in the art will understand that a composition disclosed herein is efficacious in promoting bone mineral deposition in a subject by observing that the composition increases bone mineral density (BMD). BMD can be measured by methods that are known in the art, for example, using Dual Energy X-ray Absorptiometry (DEXA).

The disclosed compositions and methods can also be used for example as tools to isolate and test new drug candidates for a variety of bone mineralization related diseases.

### C. Screening Method

Disclosed herein is a method of screening compounds to identify a TNAP activator. In general, the method involves detecting dephosphorylation of an AP substrate. For example, the method can be a chemiluminescent method of detecting substrate dephosphorylation.

1. Substrates

The AP substrate can be, for example, a 1,2-dioxetane compound. 1,2-dioxetane enzyme substrates have been well established as highly efficient chemiluminescent reporter molecules for use in enzyme immunoassays of a wide variety of types. These assays provide an alternative to conventional assays that rely on radioisotopes, fluorophores, complicated color shifting, secondary reactions and the like. Dioxetanes developed for this purpose include those disclosed in U.S. Pat. No. 4,978,614 and U.S. Pat. No. 5,112,960. U.S. Pat. No. 4,978,614 discloses, among others, 3-(2′-spiroadamantane)4-methoxy-4-(3″-phosphoryloxy)phenyl-1,2-dioxetane, which commercially available under the trade name AMPPD. U.S. Pat. No. 5,112,960, discloses dioxetane compounds, wherein the adamantyl stabilizing ring is substituted, at either bridgehead position, with a variety of substituents, including hydroxy, halogen, and the like, which convert the otherwise static or passive adamantyl stabilizing group into an active group involved in the kinetics of decomposition of the dioxetane ring. CSPD is a spiroadamantyl dioxetane phenyl phosphate with a chlorine substituent on the adamantyl group.

The AP substrate can be CSPD® (Disodium 3-(4-methoxyspiro{1,2-dioxetane-3,2′-(5′-chloro)tricyclo[3.3.1.13,7]decan}-4-yl)phenyl phosphate) or CDP-Star® (Disodium 2-chloro-5-(4-methoxyspiro{1,2-dioxetane-3,2′-(5′-chloro)-ricyclo[3.3.1.13,7]decan}-4-yl)-1-phenyl phosphate) substrates (Applied Biosystems, Bedford, Mass.). CSPD® and CDP-Star® substrates produce a luminescent signal when acted upon by AP, which dephosphorylates the substrates and yields anions that ultimately decompose, resulting in light emission. Light production resulting from chemical decomposition exhibits an initial delay followed by a persistent glow that lasts as long as free substrate is available. The glow signal can endure for hours or even days if signal intensity is low; signals with very high intensities may only last for a few hours. With CSPD® substrate, peak light emission is obtained in 10-20 min in solution assays, or in about four hours on a nylon membrane; CDP-Star® substrate exhibits solution kinetics similar to CSPD® substrate, but reaches peak light emission on a membrane in only 1-2 hours. Despite these long times to peak signal intensity, however, X-ray film exposure usually only requires 15 sec to 15 min with standard X-ray film. Both substrates provide high detection sensitivity, fast X-ray film exposure, superior band resolution, and glow light emission kinetics, enabling acquisition of multiple film exposures and use of luminometers without automatic reagent injectors. CDP-Star® substrate exhibits a brighter signal (5-10-fold) and a faster time to peak light emission on membranes, making CDP-Star® substrate the preferred choice when imaging membranes on digital signal acquisition systems.

AP substrates can be in an alkaline hydrophobic environment. Thus, substrate formulations can be in an alkaline buffer solution.

The AP substrates can be used in conjunction with enhancement agents, which include natural and synthetic water-soluble macromolecules, which are disclosed in detail in U.S. Pat. No. 5,145,772. Example enhancement agents include water-soluble polymeric quaternary ammonium salts, such as poly(vinylbenzyltrimethylammonium chloride) (TMQ), poly(vinylbenzyltributylammonium chloride) (TBQ) and poly(vinylbenzyldimethylbenzylammonium chloride) (BDMQ). These enhancement agents improve the chemiluminescent signal of the dioxetane reporter molecules, by providing a hydrophobic environment in which the dioxetane is sequestered. Water, an unavoidable aspect of most assays, due to the use of body fluids, is a natural “quencher” of the dioxetane chemiluminescence. The enhancement molecules can exclude water from the microenvironment in which the dioxetane molecules, or at least the excited state emitter species reside, resulting in enhanced chemiluminescence. Other effects associated with the enhancer-dioxetane interaction could also contribute to the chemiluminescence enhancement.

Additional advantages can be secured by the use of selected membranes, including nylon membranes and treated nitrocellulose, providing a similarly hydrophobic surface for membrane-based assays, and other membranes coated with the enhancer-type polymers described.

The disclosed reaction is 2, 3, or 4 orders of magnitude more sensitive than previously utilized colorimetric assays, a quality that allowed a decrease the concentration of TNAP, but more importantly the ability to screen in the presence of a 5-fold, 6-fold, 7-fold, 8-fold, 9-fold, or 10-fold lower concentration of diethanolamine (DEA). The luminescence signal can be linear over a 2-, 3-, or 4-orders-of-magnitude range of TNAP concentrations.

The disclosed luminescent assay can be further optimized to ensure its maximum sensitivity to compounds activating TNAP. For example, DEA buffer can be replaced with CAPS that does not contain any alcohol phosphoacceptor. This assay can provide a more accurate measure of phosphatase activity, as opposed to transphosphorylation activity that might be more relevant to in vivo conditions.

The concentration of CDP-star® can be fixed at 25 uM (˜Kₘ) to provide enough sensitivity even for compounds competitive with the CDP-star® substrate.

Half-maximal activation can correspond to 127 mM DEA. Maximal activation can result in 9.4-fold higher activity than in the absence of DEA. 600 mM DEA (pH 9.8) (e.g., in 2% DMSO) can be chosen as a positive control for TNAP activation screening. The performance of the assay can be tested in the presence and absence of DEA.

Also disclosed is a method of screening for modulators of tissue non-specific alkaline phosphatase using a colorimetric assay system, wherein the colorimetric assay system uses a phosphate-based substrate. The screening can be performed in the presence of saturating concentrations of diethanolamine. The phosphate can be p-nitrophenyl phosphate or dioxetane-phosphate.

Also disclosed is a method of identifying compounds which are capable of activating tissue non-specific alkaline phosphatase activity in animals comprising the steps of selecting compounds to be screened for activating tissue non-specific alkaline phosphatase; determining the activity of the tissue non-specific alkaline phosphatase in an in vitro assay in the presence and the absence of each compound to be screened; and comparing the activity of the tissue non-specific alkaline phosphatase in the presence and the absence of the compounds to be screened to identify compounds which are capable of activating tissue non-specific alkaline phosphatase activity in animals.

In this method, the compounds can be capable of activating the tissue non-specific alkaline phosphatase's pyrophosphatase activity. The compounds can be further administered alone for the treatment of osteoporosis in animals. Alternatively, the compounds can be administered with recombinant tissue non-specific alkaline phosphatase for the treatment of osteoporosis in animals. Similarly, the compounds can be administered alone or with recombinant tissue non-specific alkaline phosphatase to reduce the effects of hypophosphatasia in animals. The compounds can allow tapering of administration of recombinant tissue non-specific alkaline phosphatase. The compounds can serve as a means of upregulating the tissue non-specific alkaline phosphatase activity in conjunction with enzyme replacement therapy for treatment of heritable bone disorders. Alternatively, the compounds can serve as a means of upregulating the tissue non-specific alkaline phosphatase activity without using enzyme replacement therapy in animals suffering from osteoporosis. The compounds can also serve as a means of inducing higher bone mineral densities by upregulating tissue non-specific alkaline phosphatase activity or as a means of inducing higher bone mineral densities by reducing calcification inhibitors.

2. Compounds

Libraries of compounds, such as Molecular Libraries Screening Center Network (MLSCN) compounds, can be screened using the disclosed assay in search of compounds that are potent activators of TNAP. In general, candidate agents can be identified from large libraries of natural products or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, polypeptide- and nucleic acid-based compounds. Synthetic compound libraries are commercially available, e.g., from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetic libraries are produced, if desired, according to methods known in the art, e.g., by standard extraction and fractionation methods. Furthermore, if desired, any library or compound is readily modified using standard chemical, physical, or biochemical methods. In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their effect on the activity of TNAP should be employed whenever possible.

When a crude extract is found to have a desired activity, further fractionation of the positive lead extract is necessary to isolate chemical constituents responsible for the observed effect. Thus, the goal of the extraction, fractionation, and purification process is the careful characterization and identification of a chemical entity within the crude extract having an activity that stimulates or inhibits TNAP. The same assays described herein for the detection of activities in mixtures of compounds can be used to purify the active component and to test derivatives thereof. Methods of fractionation and purification of such heterogenous extracts are known in the art. If desired, compounds shown to be useful agents for treatment are chemically modified according to methods known in the art. Compounds identified as being of therapeutic value may be subsequently analyzed using animal models for diseases or conditions in which it is desirable to regulate or mimic activity of TNAP.

### D. Methods of Making the Compositions

The compositions disclosed herein and the compositions necessary to perform the disclosed methods can be made using any method known to those of skill in the art for that particular reagent or compound unless otherwise specifically noted.

### E. Definitions

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents.

It must be noted that as used herein and in the appended claims, the singular forms “a,” “an,” and “the” include plural referents unless the context clearly dictates otherwise. Thus, for example, reference to “a composition” includes mixtures of two or more such compositions, reference to “a phenylsulfamic acid” includes mixtures of two or more such phenylsulfamic acids, reference to “the compound” includes mixtures of two or more such compounds, and the like.

“Optional” or “optionally” means that the subsequently described event, circumstance, or material may or may not occur or be present, and that the description includes instances where the event, circumstance, or material occurs or is present and instances where it does not occur or is not present.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Throughout the description and claims of this specification, the word “comprise” and variations of the word, such as “comprising” and “comprises,” means “including but not limited to,” and is not intended to exclude, for example, other additives, components, integers or steps.

All percentages, ratios and proportions herein are by weight, unless otherwise specified. All temperatures are in degrees Celsius (° C.) unless otherwise specified.

By “pharmaceutically acceptable” is meant a material that is not biologically or otherwise undesirable, i.e., the material can be administered to an individual along with the relevant active compound without causing clinically unacceptable biological effects or interacting in a deleterious manner with any of the other components of the pharmaceutical composition in which it is contained.

An organic radical can have, for example, 1-26 carbon atoms, 1-18 carbon atoms, 1-12 carbon atoms, 1-8 carbon atoms, or 1-4 carbon atoms. Organic radicals often have hydrogen bound to at least some of the carbon atoms of the organic radical. One example of an organic radical that comprises no inorganic atoms is a 5, 6,7,8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamido, substituted alkylcarboxamido, dialkylcarboxamido, substituted dialkylcarboxamido, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

Substituted and unsubstituted linear, branched, or cyclic alkyl units include the following non-limiting examples: methyl (C₁), ethyl (C₂), n-propyl (C₃), iso-propyl (C₃), cyclopropyl (C₃), n-butyl (C₄), sec-butyl (C₄), iso-butyl (C₄), tert-butyl (C₄), cyclobutyl (C₄), cyclopentyl (C₅), cyclohexyl (C₆), and the like; whereas substituted linear, branched, or cyclic alkyl, non-limiting examples of which includes, hydroxymethyl (C₁), chloromethyl (C₁), trifluoromethyl (C₁), aminomethyl (C₁), 1-chloroethyl (C₂), 2-hydroxyethyl (C₂), 1,2-difluoroethyl (C₂), 2,2,2-trifluoroethyl (C₃), 3-carboxypropyl (C₃), 2,3-dihydroxycyclobutyl (C₄), and the like.

Substituted and unsubstituted linear, branched, or cyclic alkenyl include, ethenyl (C₂), 3-propenyl (C₃), 1-propenyl (also 2-methylethenyl) (C₃), isopropenyl (also 2-methylethen-2-yl) (C₃), buten-4-yl (C₄), and the like; substituted linear or branched alkenyl, non-limiting examples of which include, 2-chloroethenyl (also 2-chlorovinyl) (C₂), 4-hydroxybuten-1-yl (C₄), 7-hydroxy-7-methyloct-4-en-2-yl (C₉), 7-hydroxy-7-methyloct-3,5-dien-2-yl (C₉), and the like.

Substituted and unsubstituted linear or branched alkynyl include, ethynyl (C₂), prop-2-ynyl (also propargyl) (C₃), propyn-1-yl (C₃), and 2-methyl-hex-4-yn-1-yl (C₇); substituted linear or branched alkynyl, non-limiting examples of which include, 5-hydroxy-5-methylhex-3-ynyl (C₇), 6-hydroxy-6-methylhept-3-yn-2-yl (C₈), 5-hydroxy-5-ethylhept-3-ynyl (C₉), and the like.

The term “aryl” as used herein denotes organic rings that consist only of a conjugated planar carbon ring system with delocalized pi electrons, non-limiting examples of which include phenyl (C₆), naphthylen-1-yl (C₁₀), naphthylen-2-yl (C₁₀). Aryl rings can have one or more hydrogen atoms substituted by another organic or inorganic radical. Non-limiting examples of substituted aryl rings include: 4-fluorophenyl (C₆), 2-hydroxyphenyl (C₆), 3-methylphenyl (C₆), 2-amino-4-fluorophenyl (C₆), 2-(N,N-diethylamino)phenyl (C₆), 2-cyanophenyl (C₆), 2,6-di-tert-butylphenyl (C₆), 3-methoxyphenyl (C₆), 8-hydroxynaphthylen-2-yl (C₁₀), 4,5-dimethoxynaphthylen-1-yl (C₁₀), and 6-cyanonaphthylen-1-yl (C₁₀).

The term “heteroaryl” denotes an aromatic ring system having from 5 to 10 atoms. The rings can be a single ring, for example, a ring having 5 or 6 atoms wherein at least one ring atom is a heteroatom not limited to nitrogen, oxygen, or sulfur. Or “heteroaryl” can denote a fused ring system having 8 to 10 atoms wherein at least one of the rings is an aromatic ring and at least one atom of the aromatic ring is a heteroatom not limited nitrogen, oxygen, or sulfur.

The following are non-limiting examples of heteroaryl rings according to the present disclosure:

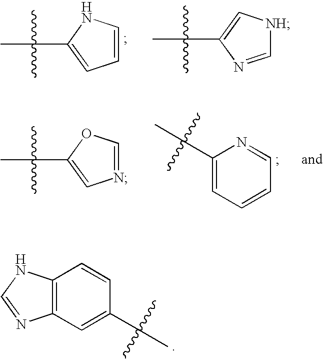

The term “heterocyclic” denotes a ring system having from 3 to 10 atoms wherein at least one of the ring atoms is a heteroatom not limited to nitrogen, oxygen, or sulfur. The rings can be single rings, fused rings, or bicyclic rings. Non-limiting examples of heterocyclic rings include:

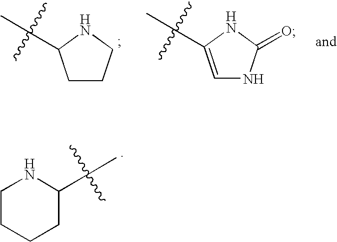

All of the aforementioned heteroaryl or heterocyclic rings can be optionally substituted with one or more substitutes for hydrogen as described herein further.

Throughout the description of the present disclosure the terms having the spelling “thiophene-2-yl and thiophene-3-yl” are used to describe the heteroaryl units having the respective formulae:

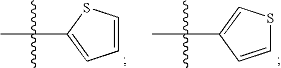

whereas in naming the compounds of the present disclosure, the chemical nomenclature for these moieties are typically spelled “thiophen-2-yl and thiophen-3-yl” respectively. Herein the terms “thiophene-2-yl and thiophene-3-yl” are used when describing these rings as units or moieties which make up the compounds of the present disclosure solely to make it unambiguous to the artisan of ordinary skill which rings are referred to herein.

The term “substituted” is used throughout the specification. The term “substituted” is defined herein as “a hydrocarbyl moiety, whether acyclic or cyclic, which has one or more hydrogen atoms replaced by a substituent or several substituents as defined herein below.” The units, when substituting for hydrogen atoms are capable of replacing one hydrogen atom, two hydrogen atoms, or three hydrogen atoms of a hydrocarbyl moiety at a time. In addition, these substituents can replace two hydrogen atoms on two adjacent carbons to form said substituent, new moiety, or unit. For example, a substituted unit that requires a single hydrogen atom replacement includes halogen, hydroxyl, and the like. A two hydrogen atom replacement includes carbonyl, oximino, and the like. A two hydrogen atom replacement from adjacent carbon atoms includes epoxy, and the like. A three hydrogen replacement includes cyano, and the like. The term substituted is used throughout the present specification to indicate that a hydrocarbyl moiety, inter alia, aromatic ring, alkyl chain; can have one or more of the hydrogen atoms replaced by a substituent. When a moiety is described as “substituted” any number of the hydrogen atoms may be replaced. For example, 4-hydroxyphenyl is a “substituted aromatic carbocyclic ring”, (N,N-dimethyl-5-amino)octanyl is a “substituted C₈alkyl unit, 3-guanidinopropyl is a “substituted C₃ alkyl unit,” and 2-carboxypyridinyl is a “substituted heteroaryl unit.”

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### F. Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### Enzyme Replacement Therapy for Hypophosphatasia

Results

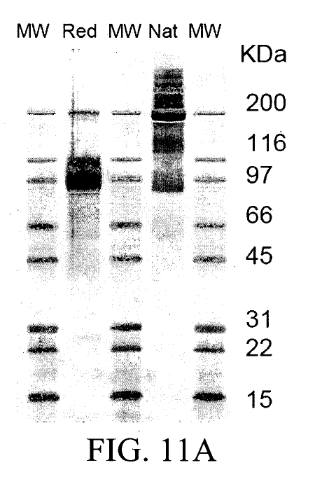

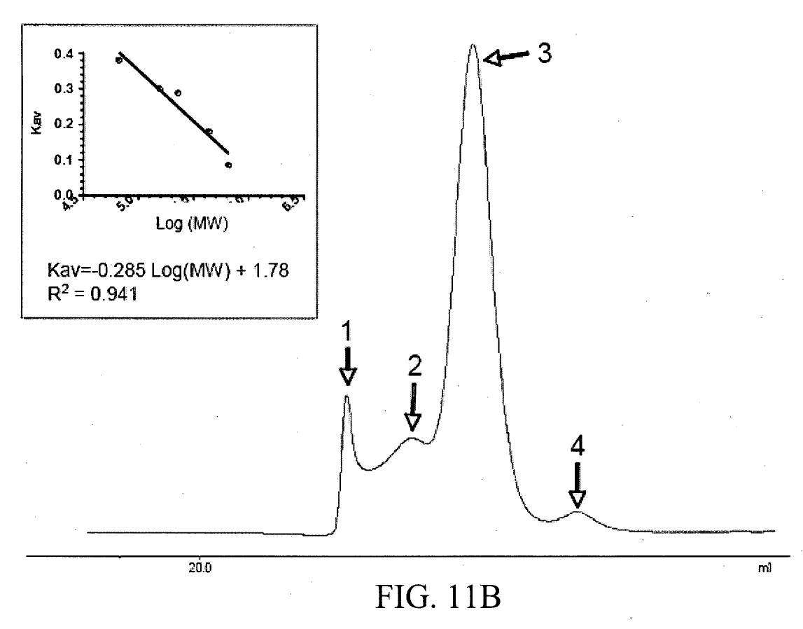

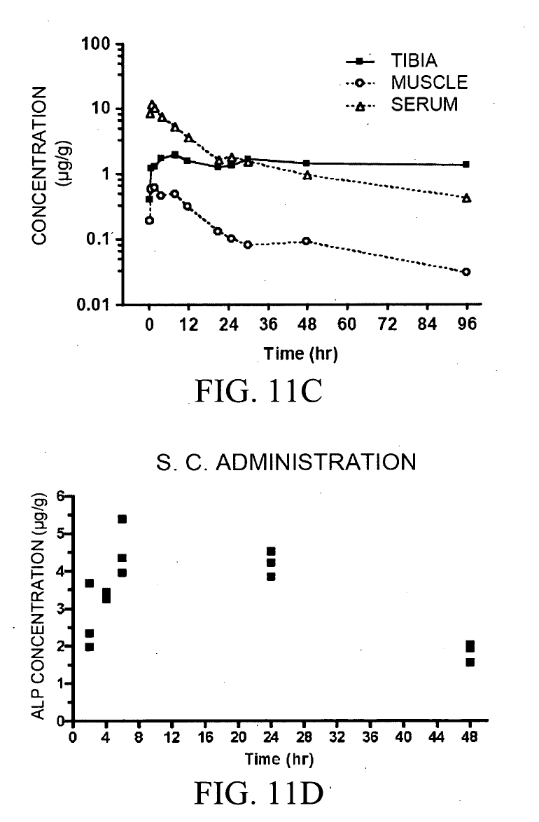

Production and characterization of sALP-FcD₁₀. To facilitate the expression and purification of recombinant TNALP, the hydrophobic C-terminal sequence that specifies GPI-anchor attachment was removed, thereby creating a soluble secreted enzyme, and the coding sequence of its ectodomain was extended with the Fc region of the human IgG (γ1 form). This allowed rapid purification of the recombinant enzyme using Protein A chromatography. Furthermore, to target the recombinant TNALP to bone tissue, a deca-aspartate (D₁₀) sequence was fused to the C-terminal of each Fc region. The fraction of sALP-FcD₁₀ protein purified on Protein-A Sepharose was analyzed on SDS-PAGE under reducing conditions where it migrated as a broad band with an apparent molecular mass of 90,000. Peptide N-Glycosidase F (PNGAse F) digestion reduced the apparent molecular mass to ˜80,000, which closely approximates the calculated mass of 80,500 Da for the non-glycosylated sALP-FcD₁₀ monomer. Using SDS-PAGE under non-reducing conditions, the apparent molecular mass of sALP-FcD₁₀ was ˜200,000 (FIG. 11A), consistent with a dimmer, as in native, unaltered, TNALP. This dimeric form of TNALP can result from two disulfide bridges in the hinge domain of two monomeric Fc regions. The molecular mass of sALP-FcD₁₀ under native conditions was approximately 370 Kd, indicating a tetrameric form for the native sALP-FcD₁₀ recombinant enzyme produced in CHO cells (FIG. 11B). The affinity of the purified sALP-FcD₁₀ protein for hydroxyapatite mineral was contrasted to that of soluble TNALP derived from bovine kidney. It was observed that sALP-FcD₁₀ binds 32-fold more efficiently to reconstituted hydroxyapatite than does bovine kidney TNALP. Furthermore, most of the recombinant sALP-FcD₁₀ protein introduced in the assay we could be account for by summing up the enzymatic activity recovered in both the bound and non-bound fractions. This indicated that binding of sALP-FcD₁₀ to mineral does not significantly alter its enzymatic activity.

Pharmacokinetic properties of sALP-FcD₁₀. Next, the pharmacokinetics (PK) and tissue distribution of sALP-FcD₁₀ was determined in adult and newborn mice comparing different routes of administration. First a single i.v. bolus of 5 mg/kg sALP-FcD₁₀ was injected into adult WT mice. The circulating half-life was 34 h, with prolonged retention of the [¹²⁵I]-labeled sALP-FcD₁₀ in bone, with as much as 1 μg/g of bone (wet) weight (Table 4 and FIG. 11C). Skeletal levels of the bone-targeted test material seemed quite stable, because no significant decrease in radiolabeled sALP-FcD₁₀ was observed during the experiment. Conversely, no sustained accumulation of sALP-FcD₁₀ was observed in muscle, as the amount of radiolabeled enzyme in this tissue decreased in parallel with sALP-FcD₁₀ enzymatic activity in blood (FIG. 11C). Because Akp2^{-/-} mice die between days 12-16 and i.v. injection was not feasible in such small animals, PK analysis of sALP-FcD₁₀ was planned using i.p. and s.c. administration in newborn WT mice. However, i.p. injection proved unreliable due to the high intraabdominal pressure in these young animals that led to unpredictable losses through the injection site. Instead, s.c. injections proved reproducible (FIG. 11D), and were followed by detection of sALP-FcD₁₀ catalytic activity in trabecular bone (FIG. 11E). PK data were then used to predict circulating levels of sALP-FcD₁₀ achieved after repeated daily s.c. injections. Circulating sALP-FcD₁₀ would reach steady state serum concentrations (C) oscillating between Cₘᵢₙ and Cₘₐₓ values of 26.4 and 36.6 μg/ml, respectively, and be achieved after 5 to 6 daily doses of 10 mg/kg. Prediction validity was tested using 5 daily s.c. injections of 10 mg/kg of sALP-FcD₁₀. Circulating ALP activity measured 24 h after the last injection (Cₘᵢₙ) was in good agreement with predicted concentrations. In WT mice, serum TNALP levels measured in the same conditions were found to be 0.58 μg/ml. Thus, it was calculated that the s.c. injection regimen would achieve steady state circulating concentrations of sALP-FcD₁₀ approximately 50 times higher than normal WT TNALP concentrations. For comparison, in the unsuccessful clinical attempts using ERT by injections of Paget's bone disease plasma, purified liver ALP, or purified placental ALP, only as much as an 8-fold elevation in serum ALP activity had been achieved.

TABLE 4
Pharmacokinetic parameters of sALP-FcD10
in newborn and adult WT mice.

| | |
|---|---|
| Newborn WT | Adult WT |

| Parameter | S.C. | I.P. | I.P. | S.C. |
|---|---|---|---|---|
| T_{1/2} (h) | 30.9 | 19.3 | 20.0 | 20.5 |
| Tₘₐₓ (h) | 6 | 6 | 4 | NA |
| Cₘₐₓ (mg/L) | 4.6 | 2.7 | 10.0 | NA |
| AUC_{inf} (mg/L/h) | 257 | 92 | 325 | 362 |
| AUC residual (%) | 35 | 20 | 20 | 18 |
| BIOAVAILABILITY (%) | 43 | 15 | 89 | |

T_{1/2} (h): Elimination half-life in hours; Cₘₐₓ: Maximal concentration; AUC: area under the curve; AUC_{inf}: area under concentration-versus-time curve to infinite; Bioavailability was expressed as percentage of AUCinf in adult WT mice after intravenous injection.

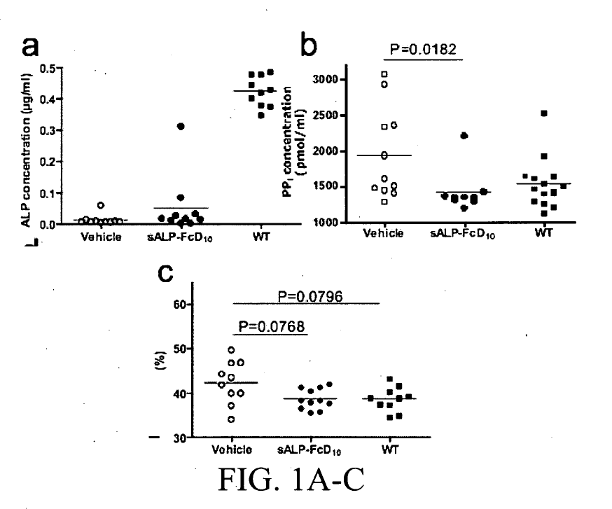

Short-term, low dose (1 mg/kg/d) treatment with sALP-FcD₁₀. The first disease efficacy study using ERT involved daily s.c. injections of sALP-FcD₁₀ for 15 days in newborn Akp2^{-/-} mice using 1 mg/kg per dose. Akp2^{-/-} mice received vehicle (n=13) (untreated) or sALP-FcD₁₀ (n=12) (treated). Healthy controls consisted of 15 WT newborn mice that were not injected. At this dose, ALP activity in plasma at day 16 in the treated Akp2^{-/-} mice was barely above the detection level (FIG. 1A). Despite the low plasma values for sALP-FcD₁₀, serum PPᵢ levels remained normal (FIG. 1B). In this short-term experiment, however, μCT analysis showed no prevention of skeletal disease in calvariae from treated Akp2^{-/-} mice at 16 days-of-life. The proximal tibial growth plates (physes) showed excessive widening of the hypertrophic zone in both sALP-FcD₁₀ and vehicle injected Akp2^{-/-} animals (FIG. 1C) consistent with early rickets. However, physeal morphology seemed less disturbed in the animals treated with sALP-FcD₁₀ for 15 days as demonstrated by Goldner's trichrome staining of representative growth plates of WT, vehicle, and treated Akp2^{-/-} mice.

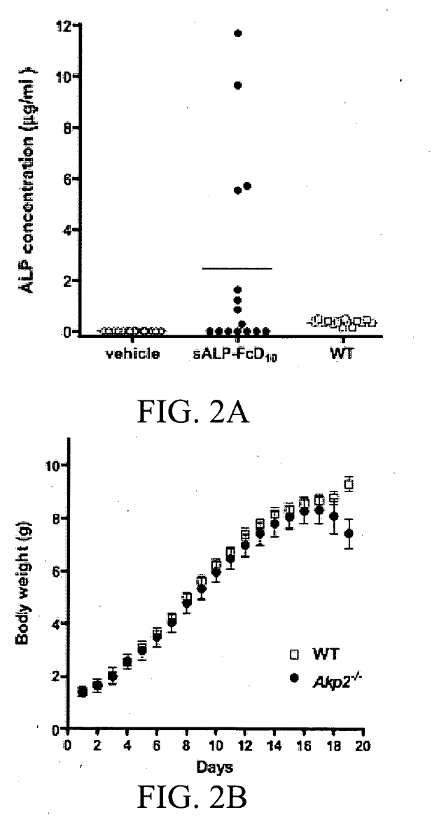

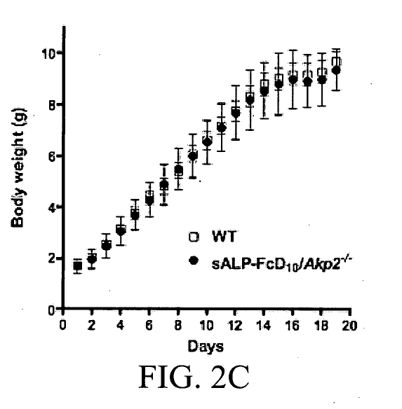

Short-term, intermediate dose (2 mg/kg/d) treatment. Next, daily s.c. injections of sALP-FcD₁₀ were given for 19 days using 2 mg/kg doses. Elevated sALP-FcD₁₀ concentrations were detected in serum from ˜50% of the treated Akp2^{-/-} mice (0.8-10.0 μg/ml in 6 of 13 mice), whereas the remaining treated mice had low, but detectable, sALP-FcD₁₀ levels (FIG. 2A). General appearance, body weight/tail length, and behavior indicated that most treated Akp2^{-/-} mice maintained their growth rate and well-being (FIG. 2C). At this dose, activity of sALP-FcD₁₀ was detected in trabecular bone by histochemical staining for ALP activity in the long bones of sALP-FcD₁₀-treated Akp2^{-/-} mice, i.e., proximal tibia of a sALP-FcD₁₀-treated mouse (2 mg/kg×24 hr) compared to the proximal tibia of age-matched untreated Akp2^{-/-} mouse.

ERT benefit was now also evident by μCT. Bone mineral density (BMD) of the spine was higher in the treated (238±37 mg/cc) versus untreated (191±13 mg/cc) Akp2^{-/-} mice (p=0.027). In the femoral cortical bone, thickness and area tended to be greater in the treated versus untreated mice: 0.11±0.16 vs 0.09±0.04 mm (p=0.064), and 0.39±0.05 vs 0.32±0.01 mm2 (p=0.054), respectively. Histomorphometry showed no differences in the bone volume fraction (BVF) or trabecular number, but there was greater trabecular thickness. Thus, greater BMD with treatment was due to thicker trabeculae. Also, sALP-FcD₁₀ preserved BMD and BVF of the proximal trabeculae in the femur, and preserved BMD as well as width and thickness of frontal and parietal calvarial bones.

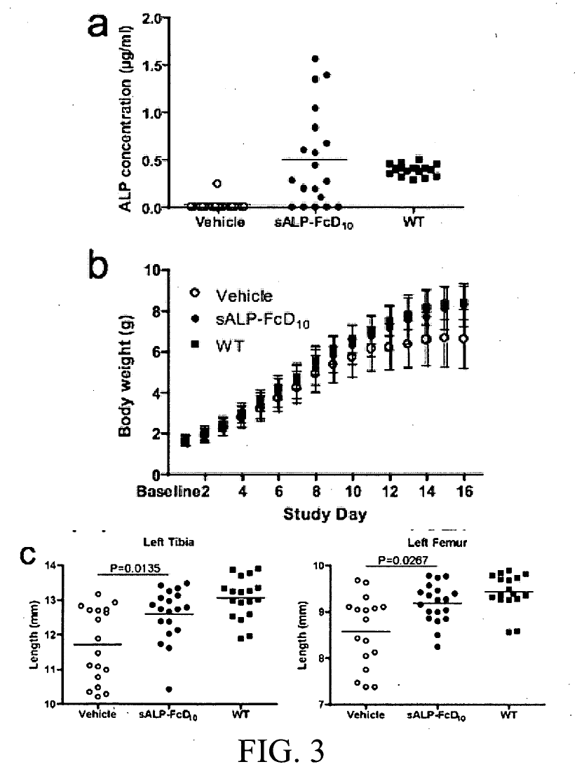

Short-term, high dose (8.2 mg/kg/day) treatment. Next, 15 days of daily s.c. injections were evaluated using the highest dose of sALP-FcD₁₀ (8.2 mg/kg). Akp2^{-/-} mice were given vehicle (n=18) or treated with sALP-FcD₁₀ (n=19). Additionally, there was one non-treated WT mouse per litter (n=18). In all but 5 treated Akp2^{-/-} mice, detectable, but highly variable, levels of sALP-FcD₁₀ were found in the plasma (FIG. 3A). Circulating TNALP concentrations in WT mice are given for comparison. sALP-FcD₁₀-treated animals had greater body weight than vehicle-treated mice and were undistinguishable from WT mice (FIG. 3B) and had plasma PPᵢ concentrations in the normal range when the experiment ended.

At completion (day 16), tibia and femur lengths provided additional measures of skeletal benefit for the ERT mice: for tibias, treated 12.6±0.7 mm versus vehicle 11.7±1.1 mm (p=0.014) (FIG. 3C); for femurs, treated (9.2±0.4 mm) versus vehicle (8.6±0.8 mm) (p=0.027). Using “blinded” evaluations of Faxitron x-ray images of the feet and rib cages, two degrees of severity of mineralization defects appeared distinguishable in the Akp2^{-/-} mice (see Table 5). Severely affected mice (Severe) had absence of digital bones (phalanges) and secondary ossification centers. Moderately affected (Moderate) mice had abnormal secondary ossification centers, but all digital bones were present. WT mice (Healthy) had all bony structures present with normal architecture. Radiographic images of the hind limbs were similarly classified as abnormal if evidence of acute or chronic fractures was present, or healthy in the absence of any abnormal findings.

TABLE 5
Faxitron image distribution table

| | | Vehicle | sALP-FcD₁₀ | WT |
|---|---|---|---|---|
| Feet | Healthy | 3 | 2 | 16 |
| | Moderate | 10 | 17 | 2 |
| | Severe | 5 | 0 | 0 |
| Ribs | Healthy | 1 | 2 | 17 |
| | Moderate | 11 | 17 | 1 |
| | Severe | 6 | 0 | 0 |
| Legs | Healthy | 9 | 16 | 18 |
| | Abnormal | 9 | 0 | 0 |

ERT minimized hypomineralization defects in the feet documented by the number of Akp2^{-/-} mice with severe defects, consisting of 5 in the untreated group yet none in the ERT group (Table 5). Chi-Square was significant (p≦0.05), indicating ERT decreased the severity of the acquired bone defects. Because severely affected infantile HPP patients often die from undermineralized and fractured ribs incapable of supporting respiration, the thoraces were also closely examined. ERT also reduced the incidence of severely dysmorphic rib cages (Table 5). Chi-Square analysis was significant at p≦0.025. Similarly, the hind limbs appeared healthy in all treated animals (Table 5). Chi-Square analysis was significant at p≦0.025.

The extent of dental abnormalities was also examined in vehicle and sALP-FcD₁₀-treated mice compared to WT controls. In the Akp2^{-/-} mice, the incisor root analogue dentin and the molar root dentin were particularly sensitive to the lack of TNALP, and only partially mineralize. Extensive regions of unmineralized crown analogue dentin and unmineralized root analogue dentin were present. Likewise, the surrounding alveolar bone also showed regions of unmineralized bone matrix. sALP-FcD₁₀ treatment preserved mineralization at the histological level in both alveolar mandibular bone and in teeth (incisor and molar). sALP-FcD₁₀ treatment of Akp2^{-/-} mice enabled complete mineralization of all incisor tooth tissues, all molar dentin, and surrounding alveolar bone such that no mineralization differences were seen between the incisor teeth or molar teeth and bone of the treated mice compared to WT mice.

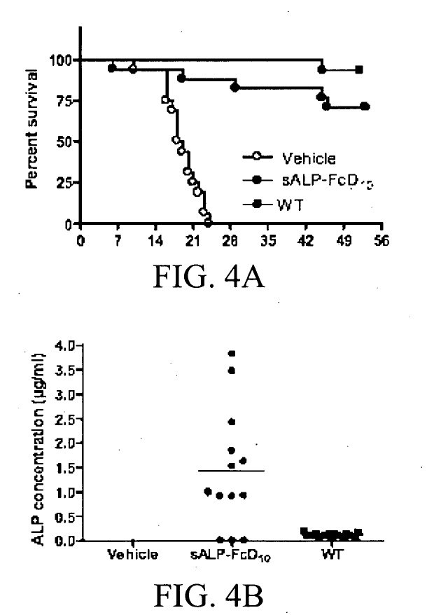

Long-term, high dose treatment. Finally, to assess the effects of ERT on long-term survival and skeletal mineralization, either sALP-FcD₁₀ (8.2 mg/kg) or vehicle was given s.c. daily to Akp2^{-/-} mice for 52 days. Untreated mice had a median survival of 18.5 days, whereas survival in sALP-FcD₁₀-treated mice was dramatically maintained while preserving normal activity and a healthy appearance (FIG. 4A). This preservation of apparent well being with EzRT was accompanied by normal plasma pyridoxal (PL) concentrations (2.9±1.1 μM in Akp2−/−-treated versus 3.0±1.4 μM in WT mice) and unremarkable calcium concentrations (1.07±0.28 mM in Akp2−/−-treated versus 1.10±0.24 mM in WT mice).

Plasma ALP activity was measured in treated and untreated Akp2^{-/-} mice at the study conclusion (FIG. 4B). Most concentrations were between 1 and 4 μg/ml of sALP-FcD₁₀. While radiographs of the hind limb of 18-day-old untreated Akp2^{-/-} mice showed disappearance of secondary ossification centers, a hallmark of human and murine HPP (21, 35), these defects were absent in sALP-FcD₁₀-treated mice at 46 or 52 days.

These findings demonstrate that sustained delivery of bone-targeted TNALP can prevent the major sequelae of infantile hypophosphatasia in Akp2^{-/-} mice. These observations represent the first successful use of ERT for a heritable primary disease of the skeleton, and are a foundation for therapeutic trials for hypophosphatasia patients.

Materials and Methods

Bioengineering and expression of recombinant sALP-FcD₁₀: The sALP-FcD₁₀ protein contains recombinant human soluble TNALP (sALP), the constant region of human IgG1 Fc domain (Fc), and a deca-aspartate motif (D₁₀). The cDNA encoding the fusion protein was inserted into the pIRES vector (Clontech, San Diego, Calif.) in the first multiple cloning site located upstream of the IRES using NheI and BamHI endonuclease restriction sites. The dihydrofolate reductase (DHFR) gene was inserted into the second multiple cloning site located downstream of the IRES using SmaI and XbaI endonuclease restriction sites. The resulting vector was transfected into Chinese Hamster Ovary (CHO-DG44) cells lacking both DHFR gene alleles using the Lipofectamine transfection kit (Invitrogen, San Diego, Calif.). Two days after transfection, media was changed and the cells were maintained in a nucleotide-free medium (IMDM supplemented with 5% dialyzed FBS) for 15 days to isolate stable transfectants for plaque cloning. Cells from three clones growing in the nucleotide-free medium were pooled and further cultivated in media (IMDM+5% dialyzed FBS) containing increasing concentrations of methotrexate (MTX). Cultures resistant to 50 nM MTX were further expanded in Cellstacks (Corning) containing IMDM medium supplemented with 5% FBS. Upon reaching confluency, the cell layer was rinsed with Phosphate Buffered Saline (PBS), and the cells were incubated for three additional days with IMDM containing 3.5 mM sodium butyrate to increase protein expression. At the end of the culture, the concentration of sALP-FcD₁₀ in the spent medium was 3.5 mg/l as assessed by TNALP enzymatic activity. Culture supernatant was then concentrated and dialyzed against PBS using tangential flow filtration and loaded on to Protein A-Sepharose columns (Hi-Trap 5 ml, GE Health Care) equilibrated with PBS. Bound proteins were eluted with 100 mM citrate pH 4.0 buffer. Collected fractions were immediately adjusted to pH 7.5 with 1 M Tris pH 9.0. Fractions containing most of the eluted material were dialyzed against 150 mM NaCl, 25 mM sodium PO₄ pH 7.4 buffer containing 0.1 mM MgCl₂, 20 μM ZnCl₂, and filtered through a 0.22 μm (Millipore, Millex-GP) membrane under sterile conditions. The overall yield of the purification procedure was 50%, with purity surpassing 95% as assessed by Sypro ruby stained SDS-PAGE. Purified sALP-FcD₁₀ preparations were stored at 4° C., and remained stable for several months.

Labeling of sALP-FcD₁₀: An aliquot containing 4 mg of sALP-FcD₁₀ was iodinated with IODO-BEADS (Pierce) according to the manufacturer's instructions. The final iodination mix contained 2 IODO-BEADS in a total volume of 2.5 ml of iodination buffer (150 mM NaCl, 25 mM Na phosphate, pH 7.4). Reaction was initiated by the addition of 1 mCi Na[¹²⁵I] and left to proceed at room temperature for 5 min before quenching with 25 μl 1.85×10⁻³ M NaI and desalting on a PD-10 column (Pharmacia). Total specific radioactivity of the labeled enzyme was approximately 50,000 dpm/μg. The specific activity of the enzyme after labeling was at least 95% that of the unlabeled enzyme.

Binding of sALP-FcD₁₀ to hydroxyapatite: sALP-FcD₁₀ and bovine kidney TNALP were compared in a reconstituted mineral-binding assay. For this experiment, hydroxyapatite ceramic beads were first solubilized in 1 M HCl and the mineral was precipitated by bringing back the solution to pH to 7.4 with 10 N NaOH. Binding to this reconstituted mineral was studied by incubating aliquots of the mineral suspension containing 750 μg of mineral with 5 μg of protein in 100 μl of 150 mM NaCl, 80 mM sodium phosphate pH 7.4, buffer. The samples were kept at 21±2° C. for 30 minutes on a rotating wheel. Mineral was spun down by low speed centrifugation and total enzymatic activity, recovered in both the mineral pellet and the supernatant, was measured. Total activity was the sum of the enzymatic activity recovered in the free and bound fractions, and was found to be 84% and 96% of enzymatic activity introduced in each set of assays for the bovine and sALP-FcD₁₀ forms of enzyme, respectively. Results are the average of two bindings. Hence, the sALP-FcD₁₀ bound to mineral with far greater affinity.

Mouse model of Infantile HPP: The Akp2^{-/-} mice, created by insertion of the Neo cassette into exon 6 of the mouse TNALP gene (Akp2) via homologous recombination, functionally inactivate the Akp2 gene resulting in no detectable TNALP mRNA or protein. Phenotypically, Akp2^{-/-} knockout mice closely mimic infantile HPP. Like HPP patients, Akp2^{-/-} mice have global deficiency of TNALP activity, endogenous accumulation of the ALP substrates PPi, PLP, and PEA, and postnatally manifest an acquired defect in mineralization of skeletal matrix leading to rickets or osteomalacia. They have stunted growth and develop radiographically and histologically apparent rickets together with epileptic seizures and apnea, and die between postnatal days 10-12. Hypercalcemia, which occurs in some severely affected HPP patients, was documented in some studies. This can be a result of failure of mineral uptake by the skeleton together with skeletal demineralization. Pyridoxine supplementation briefly suppresses the seizures in these mice, and extends their lifespan, but only until postnatal days 18-22. Therefore, all animals (breeders, nursing mothers and their pups, and weanlings) were given free access to modified laboratory rodent diet 5001 containing increased levels (325 ppm) of pyridoxine. To identify Akp2^{-/-} homozygotes at day 0 (date of birth), 0.5 μl of whole blood was used obtained at the time of toe clipping and measured serum ALP activity in a total reaction volume of 25 μl, velocity of 30 min. at OD405, with 10 mM p-nitrophenyl phosphate (pNPP). The genotype of the animals was confirmed by PCR and/or Southern blotting using tail DNA obtained at the time of tissue collection.

ALP assay: Non-fasting blood was collected by cardiac puncture into lithium heparin tubes (VWR, #CBD365958), put on wet ice for a maximum of 20 minutes, and then centrifuged at 2,500×g for 10 min at room temperature. At least 15 μl of plasma was transferred into 0.5 ml tubes (Sarstedt, #72.699), frozen in liquid N₂, and kept at −80° C. until assayed for ALP activity and PPi concentrations. Any remaining plasma was pooled with the 15 μl aliquot, frozen in liquid N₂, and kept at −80° C. Levels of sALP-FcD₁₀ in plasma were quantified using a colorimetric assay for ALP activity where absorbance of released p-nitrophenol is proportional to the reaction products. The reaction occurred in 100 μl of ALP buffer (20 mM Bis Tris Propane (HCl) pH 9, 50 mM NaCl, 0.5 mM MgCl₂, and 50 μM ZnCl₂) containing 10 μl of diluted plasma and 1 mM pNPP. The latter compound was added last to initiate the reaction. Absorbance was recorded at 405 nm every 45 seconds over 20 minutes using a spectrophotometric plate reader. sALP-FcD₁₀ catalytic activity, expressed as an initial rate, was assessed by fitting the steepest slope for 8 sequential values. Standards were prepared with varying concentrations of sALP-FcD₁₀ and ALP activity was determined as above. The standard curve was generated by plotting Log of the initial rate as a function of the Log of the standard concentrations. sALP-FcD₁₀ concentration in the different plasma samples was read from the standard curve using their respective ALP absorbance. Activity measures were transformed into concentrations of sALP-FcD₁₀ by using a calibration curve obtained by plotting the activity of known concentrations of purified recombinant enzyme.

PPi assay: Circulating levels of PPi were measured using plasma and differential adsorption on activated charcoal of UDP-D-[6-³H]glucose (Amersham Pharmacia) with the reaction product of 6-phospho[6-³H]gluconate, as previously described.

Vitamin B6 assays: Pyridoxal 5′-phosphate (PLP) and pyridoxal (PL) concentrations in plasma were measured by HPLC as described.

Plasma calcium: Plasma total calcium was measured using the ortho-cresolphtalein complexone method.

Skeletal and dental tissue preparation and morphological analysis: After anesthesia with Avertin and blood collection using exsanguination, soft tissue was dissected away and bones were fixed in 4% paraformaldehyde in PBS for 3 days and then washed in a series of sucrose (10, 15, 20%)/PBS mixtures containing 1 mM MgCl₂ and 1 mM CaCl₂ at 4° C. Bones embedded in optimal cutting temperature (OCT) compound were sectioned using a Leica CM1800 cryostat. Sections (˜9 mm) were vacuum dried for 1 hr, immediately washed in PBS, and then transferred to freshly prepared staining mixture of Naphtol AS-MX phosphate disodium salt and Fast Violet B salt (Sigma, St. Louis, Mo.) as described. Methyl green (0.0001%) served as counter stain.

Proximal tibiae were separated using a slow-speed saw. The specimens were dehydrated through a series of ascending ethanol solutions, cleared with xylene, infiltrated with methylmethacrylate, and embedded in methylmethacrylate/catalyst. Frontal sections, through the middle of the tibia, were obtained using a rotary microtome (Model RM2165, Leica Microsystems Inc., Bannockburn, Ill.). One 4 μm section was stained with Goldner's trichrome stain.

Mandibles from 16-day-old mice were immersion-fixed overnight in sodium cacodylatebuffered aldehyde solution and cut into segments containing the first molar, the underlying incisor, and the surrounding alveolar bone. Samples were dehydrated through a graded ethanol series and infiltrated with either acrylic (LR White) or epoxy (Epon 812) resin, followed by polymerization of the tissue-containing resin blocks at 55° C. for 2 days. Thin sections (1 μm) were cut on an ultramicrotome using a diamond knife, and glass slide-mounted sections were stained for mineral using 1% silver nitrate (von Kossa staining, black) and counterstained with 1% toluidine blue. Frontal sections through the mandibles (at the same level of the most mesial root of the first molar) provided longitudinally sectioned molar and cross-sectioned incisor for comparative histological analyses.

X-ray analysis: Radiographic images were obtained with a Faxitron MX-20 DC4 (Faxitron X-ray Corporation, Wheeling, Ill.), using an energy of 26 kV and an exposure time of 10 seconds.

μCT Analysis: Formalin-fixed lumbar vertebrae, femora, and calvaria were analyzed for bone architecture using the MS-8 system (GE Healthcare, London, ON) and isotropic voxel resolution of 18 μm. In each scan, a calibration phantom including air, water, and a mineral standard material (SB3, Gammex RMI) enabled calibration and conversion of X-ray attenuation such that mineral density was proportional to grayscale values in Hounsfield Units. Digital reconstruction of ray projection to CT volume data was accomplished with a modified Parker algorithm. After reconstruction, images were “thresholded” automatically to distinguish bone voxels using a built-in algorithm of the GE-supplied MicroView software. Bone mineral density (BMD; mg/cc), trabecular thickness (Tb.Th.; mm), and the number of trabeculae (Th.N.; mm⁻³) were measured in the trabecular bone region of the centrum (body) of the L2 vertebra. The region of interest (ROI) was defined as an elliptical cylinder with dimensions 0.45 mm×1.0 mm×0.9 mm. Care was taken to exclude cortical bone from these measurements. The trabecular bone volume fraction (BVF) was calculated as the number of bone voxels divided by the total number of voxels (BV/TV) within the ROI. BMD was also measured in the parietal region of the calvaria with the ROI defined as a cube that enclosed a 3 mm wide segment of the parietal bone. Cortical bone thickness and area were measured in the femur with the ROI defined as a 1.0 mm long segment at mid-diaphysis.

Pharmacokinetic analysis: The WinNonlin™ 5.2 software package (Pharsight Corporation, Mountain View, Calif.) was used to predict the circulating blood levels of sALP-FcD₁₀ after repeated injections.

Statistical analysis: Non-parametric analyses were preferred for all parameters because of the small sample sizes. The Log-Rank test was used to compare survival curves. Chi-square was performed to test the distribution of radiographic severity between treatment with sALP-FcD₁₀ and vehicle. The Kruskal-Wallis Test was used to compare changes in body weights between the 3 groups of mice at each day. The Wilcoxon Two-sample Rank Sum Test or the Mann Whitney Rank Sum Test were performed to compare two sets of treatments.

### 2. Example 2

### Upregulation of TNAP Activity Increases Bone Mineral Density in Mice

As seen above, the rickets and osteomalacia characteristic in tissue-nonspecific alkaline phosphatase (TNAP)-deficient mice (Akp2^{-/-} mice) results from highly increased levels of the calcification inhibitor PPᵢ, a natural substrate of TNAP. These studies indicated the possibility of manipulating PPᵢ concentrations as a means of affecting calcification. Thus, transgenic mice over-expressing TNAP might be able to achieve tissular expression of TNAP sufficiently high to be able to lower circulating PPᵢ concentrations to enhance bone mineral density (BMD) in these animals. Transgenic mice were generated by expressing human TNAP cDNA under control of the Apolipoprotein E promoter, which drives expression of TNAP primarily in the post-natal liver. The expression levels of TNAP were examined in tissues from mice carrying one copy or two copies of the ApoE-Tnap transgene and also from [Akp2^{-/-}; ApoE-Tnap] mice, and the ability of their primary osteoblasts to calcify in culture examined. Staining indicates expression of mouse TNAP (Akp2) in wild-type samples, and expression of human transgene (ApoE-Tnap) in the transgenic samples (11-day-old). See Table 6 for results.

TABLE 6
Alkaline phosphatase staining

| | | Wild-type | Akp2^{-/-}; ApoT(+) |
|---|---|---|---|
| Head | Calvarial bone | +++ | + |
| | Cerebrum | − | + |
| | Midbrain, -pons | − | +++ |
| | Cerebellum | − | +++ |
| | Medulla/pons | − | +/− |
| | Spinal cord | +/− | + |
| | Choroids plexus | +++ | − |
| Upper body | Fat | +++ capillaries | − |
| | Thymus | − | ++ unknown cells |
| | Ganglions | + | − |
| | Muscle | +++ capillaries | ++ unknown cells |
| | Lymph node | − | ++ outer layer |
| | Hair follicle | + | − |
| | Lung | − | + |
| | Heart | − | − |
| Internal | Liver | − | +++ |
| organs | Instestines | +++ | +++ |
| | Spleen | + | + unknown cells |
| | Large intestine | + | + |
| Lower body | Kidney | +++ | +++ |
| | Adrenal gland | − | +++ |
| | Testis | + peritublar | − |
| Hind limbs | Epiphyseal | +++ | + |
| | Trabecular | +++ | + |
| | Diaphyseal | +++ | + |
| | Hypertrophic | +++ | − |
| | chondrocytes | | |

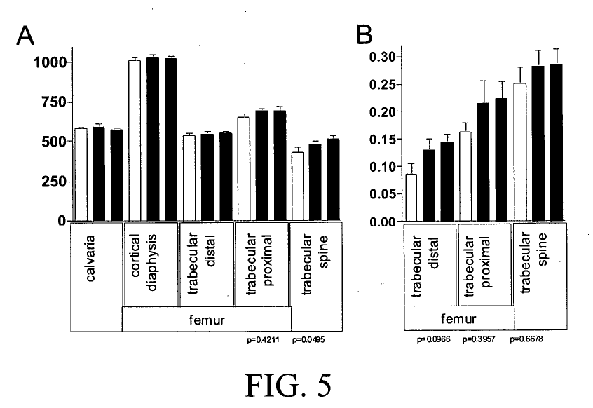

MicroCT analysis was used to measure BMD in long bones, vertebrae and calvaria (FIG. 5). TNAP expression in ApoE-Tnap mice was major in the liver and kidney, with lower but yet detectable levels in bone, brain and lung. Serum AP concentrations were 10 to 50-fold higher than age-matched sibling control wild-type (WT) mice. Serum levels of PPᵢ were reduced in the transgenic animals. Furthermore, μCT analysis of femur, vertebrae and calvaria revealed higher BMD in cancellous bone of ApoE-Tnap⁺ and ApoE-Tnap^{+/+} mice compared to WT mice.

Thus, increases in tissular and circulating levels of TNAP lead to higher BMD by reducing the effective levels of the calcification inhibitors PPᵢ and OPN. These data provide a mechanistic interpretation for the correlation between AP and BMD that has been observed in humans and mice. Furthermore, these studies support that administering recombinant TNAP can serve as a therapeutic approach for osteoporosis.

### 3. Example 3

### Identification of TNAP Activators

The majority of mechanistic studies on alkaline phosphatases have been performed on E. coli alkaline phosphatase. This information is directly applicable to the mammalian alkaline phosphatases due to high degree of sequence and structure homology. All alkaline phosphatases exist as homodimers, and oligomerization is required for their catalytic activity. The alkaline phosphatases catalyze hydrolysis of phosphate monoesters and this proceeds through a phosphoserine covalent intermediate. The detailed mechanism of a general alkaline phosphatase reaction is outlined below.

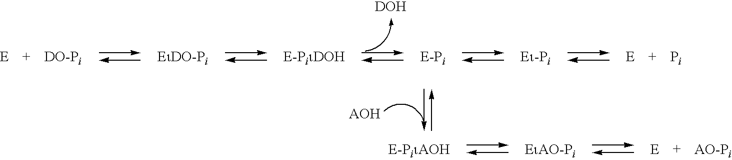

The above schematic shows the catalytic mechanism of alkaline phosphatase reaction (Millán, 2006). The initial alkaline phosphatase (E) catalyzed reaction consists of a substrate (DO-Pi) binding step, phosphate-moiety transfer to Ser-93 (in the TNAP sequence of its active site) and product alcohol (DOH) release. In the second part of the reaction, phosphate is released through hydrolysis of the covalent intermediate (E-Pᵢ) and non-covalent complex (E.Pᵢ) of inorganic phosphate in the active site. In the presence of alcohol molecules (AOH), phosphate is released via a transphosphorylation reaction.

Inorganic pyrophosphate (PPᵢ) and pyridoxal-5′-phosphate (PLP), a form of vitamin B6, are the endogenous substrates for TNAP. As with other alkaline phosphatases, the hydrolysis of the phosphoserine intermediate is the rate limiting step of the TNAP overall reaction and consequently its acceleration would lead to an increase in the TNAP turnover rate. This acceleration lies at the heart of the molecular mechanism of alkaline phosphatases, known as the flip-flop mechanism (Lazdunski et al., 1971). According to this mechanism, two subunits within a dimer act in an interdependent fashion with catalysis in one subunit promoting the catalysis in the second subunit. Interestingly, binding of a competitive inhibitor to one of the subunits was shown to accelerate the rate of phosphate release from the second subunit (Lazdunski et al., 1971). This indicates that activity of alkaline phosphatases not only can be inhibited but also activated through small molecule interactions within the active site.

The activity of active site variants D101S and D153G of the E. coli enzyme (Holtz and Kantrowitz, 1999), and E108A (Kozlenkov et al., 2004) and A160T (Di Mauro et al., 2002) of human TNAP were reported to be 35-, 5-, 2- and 2-fold higher than that of the corresponding wild-type enzymes, respectively. This shows that certain patterns of interference with the hydrogen-bonding network within the active site of an alkaline phosphatase would result in enzyme activation. In the presence of certain alcohol molecules, such as diethanolamine (DEA) or Tris, the rate-limiting step of phosphoserine hydrolysis is bypassed with a faster transphosphorylation step resulting in significant acceleration of turnover rate as will be illustrated below (FIG. 10). Inorganic phosphate exhibits product inhibition of the TNAP reaction. Therefore, the in vivo reaction of TNAP is negatively regulated by its product concentration. Spatial or electrostatic hindrance that could result from small molecule binding in the vicinity of the active site can lead to relief of product inhibition and an increase in the overall turnover rate of pyrophosphate hydrolysis. In the search for inhibitors of TNAP, the LoPAC¹²⁸⁰, Spectrum and Chembridge DIVERSet collections (53,280 compounds total) were screened using a calorimetric assay using p-nitrophenyl phosphate as the substrate. A number of positive hits were identified and confirmed (Narisawa et al, submitted). This screening was performed in the presence of saturating concentrations of DEA to provide maximal activity of the enzyme.

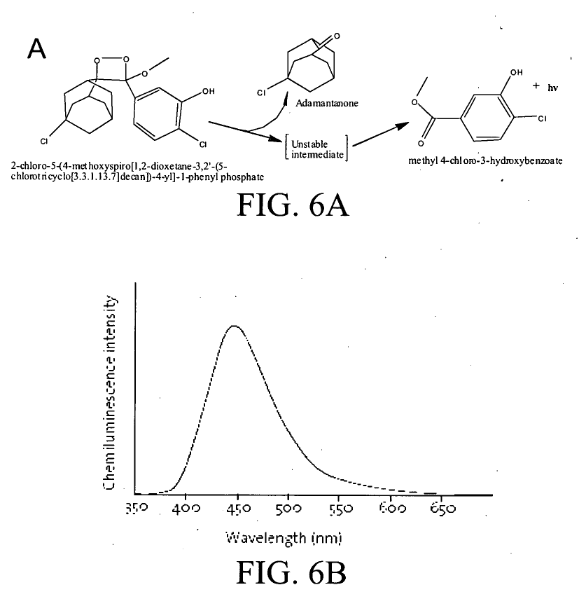

Moving to the next stage of screening, one concern was that the phosphoacceptor binding site could not be targeted. To address this issue, a TNAP assay was developed. This assay is based on the dephosphorylation of a CDP-star® alkaline phosphatase substrate (New England Biolabs, Inc.) designed to detect alkaline phosphatase in blotting techniques (FIG. 6). As with many chemiluminescent reactions, the dioxetane-based reaction represents a sequence of several steps. Dioxetane-phosphate is dephosphorylated by an alkaline phosphatase leading to the generation of an unstable product that decomposes to a stable product with concomitant light production. Once the steady-state of the overall reaction is achieved (in the current reaction it happens within the first 5 min), the luminescence signal output is stable over several hours. As an added bonus, the light intensity of the chemiluminescent reaction is directly proportional to the rate of the TNAP reaction; therefore, the activity of the enzyme can be reliably measured in real-time.

This reaction is four orders of magnitude more sensitive than the previously utilized colorimetric assay, a quality that allowed a decrease the concentration of TNAP, but more importantly the ability to screen in the presence of a 10-fold lower concentration of DEA. The luminescence signal was linear over a four-orders-of-magnitude range of TNAP concentrations. The cost of screening was only marginally increased (ca. 1¢/well), a circumstance that was fully outweighed by both the reduction in the number of steps involved in the assay and the associated increase in screening throughput. The full MLSMR collection (at the time 65K compounds) was screened vs TNAP in 384-well format and the screening data were deposited into PubChem (AID 518). This resulted in the identification of several structural classes of inhibitors, with the majority of compounds demonstrating apparent competition with DEA. The compounds can be further characterized using a panel of homologous human alkaline phosphatases in the presence of their artificial and natural substrates to further define the specificity of the compounds.

In addition to the extended linearity range, screening with the novel luminescent assay led to the identification of several compounds that activate the CDP star-based TNAP reaction (Table 7). In this table, the column corresponding to TNAP (luminescence) was calculated as (100−X)/100, where X is % inhibition. In some aspects, TNAP activation fact refers to the fold increase in TNAP activity. There are two tiers of data confirmation embedded in the data. First, the activation effect for replicate wells is in very good correspondence. Second, some compounds have similar structural characteristics, for example compounds 6, 20, and 28 or compounds 14, 24, 27. Taken together with the fact that the colorimetric assay is performed in the presence of a high concentration of DEA this can indicate that most of the compounds bind in the vicinity of phosphoacceptor binding site. The absence of hydroxyl groups in most of these compounds indicates that they should not act through a transphosphorylation reaction.

TABLE 7
Compounds from the MLSMR displaying apparent activation of TNAP. Compounds from
the MLSMR displaying apparent activation of TNAP.

| | | | | | | TNAP |
|---|---|---|---|---|---|---|
| | | Compound | PubChem | | | Activation |
| # | Structure | ID | SID | MolWt | ClogP | Factor^{†} |
| 1 | 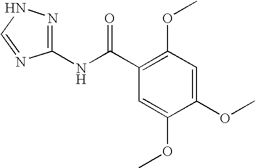 | MLS-0063500 | 7969975 | 316.34 | 0.271 | 15.4 |
| 2 | 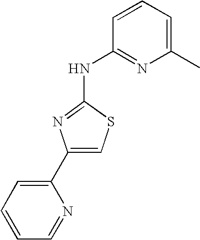 | MLS-0031960 | 846873 | 477.48 | 1.826 | 6.1 |
| 3 | 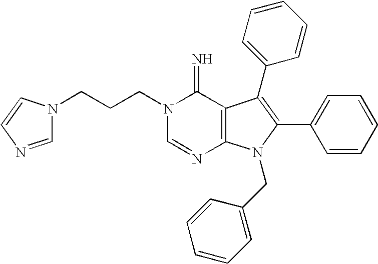 | MLS-0039243 | 858577 | 318.37 | 3.059 | 4.3 |
| 4 | 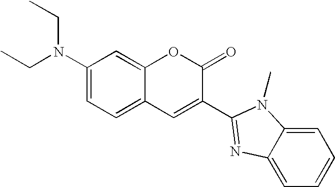 | MLS-0048724 | 4243880 | 446.54 | 5.213 | 2.8 |
| 5 | 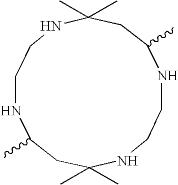 | MLS-0036044 | 7972491 | 350.09 | 4.697 | 2.3 |
| 6 | 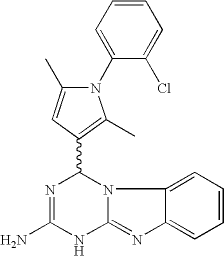 | MLS-0003213 | 855623 | 416.13 | 5.645 | 2.2 |
| 7 | 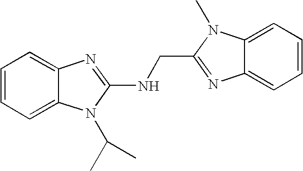 | MLS-0022403 | 864475 | 281.35 | 4.03 | 2.0 |
| 8 | 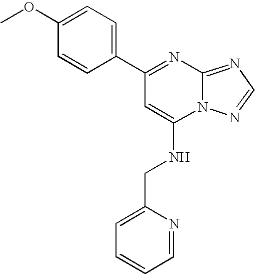 | MLS-0057682 | 853131 | 332.36 | 3.167 | 2.0 |
| 9 | 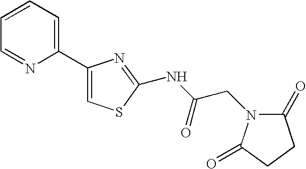 | MLS-0036195 | 7977308 | 284.48 | 0.455 | 2.0 |
| 10 | 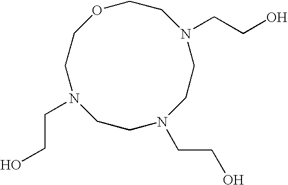 | MLS-0037367 | 3711730 | 331.41 | 3.856 | 1.9 |
| 11 | 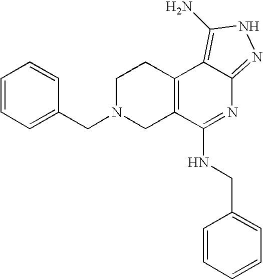 | MLS-0056994 | 3713120 | 390.87 | 4.697 | 1.9 |
| 12 | 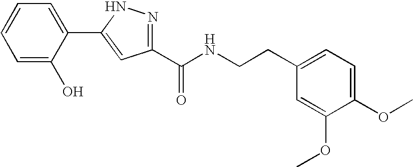 | MLS-0046246 | 4259391 | 319.4 | 3.653 | 1.9 |
| 13 | 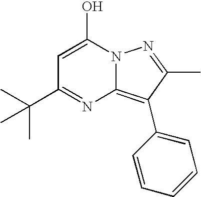 | MLS-0046514 | 864391 | 314.41 | 3.64 | 1.8 |
| 14 | 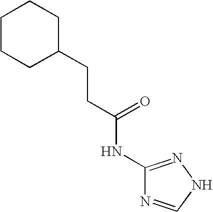 | MLS-0012821 | 7973907 | 364.42 | 3.588 | 1.8 |
| 15 | 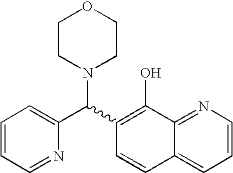 | MLS-0037240 | 4254500 | 268.34 | 2.635 | 1.7 |
| 16 | 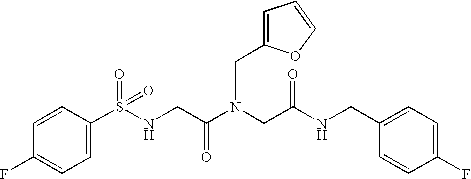 | MLS-0052784 | 4247536 | 216.24 | 1.386 | 1.7 |
| 17 | 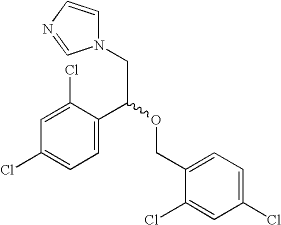 | MLS-0043212 | 7978390 | 347.41 | 4.366 | 1.7 |
| 18 | 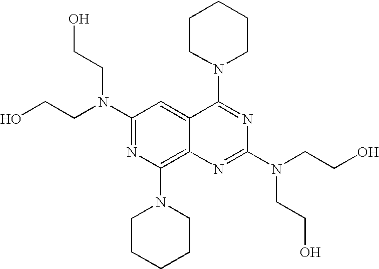 | MLS-0046482 | 3712467 | 411.48 | 4.992 | 1.6 |
| 19 | 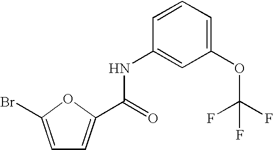 | MLS-0031679 | 3715018 | 321.37 | 2.319 | 1.6 |
| 20 | 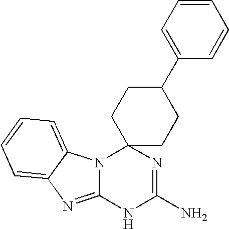 | MLS-0026826 | 4260631 | 222.29 | 2.059 | 1.6 |
| 21 | 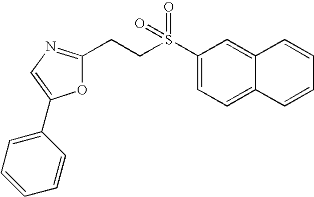 | MLS-0047819 | 4243914 | 474.64 | 4.868 | 1.6 |
| 22 | 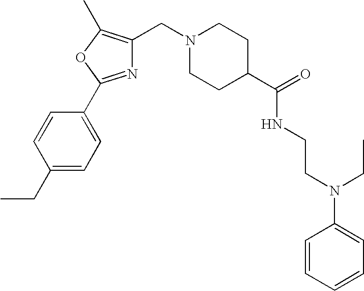 | MLS-0004128 | 4261371 | 278.26 | 0.846 | 1.6 |
| 23 | 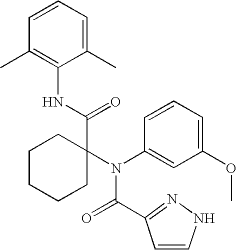 | MLS-0017060 | 865642 | 367.4 | 3.35 | 1.6 |
| 24 | 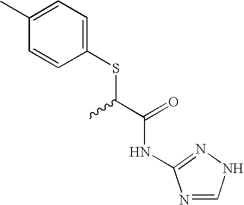 | MLS-0001325 | 856075 | 384.48 | 3.224 | 1.6 |
| 25 | 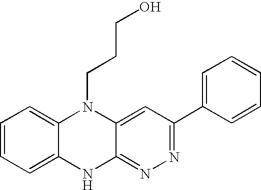 | MLS-0044127 | 4264118 | 262.33 | 2.259 | 1.5 |
| 26 | 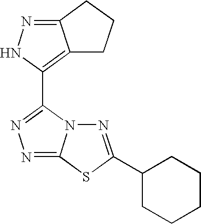 | MLS-0041244 | 846813 | 305.41 | −1.333 | 1.5 |
| 27 | 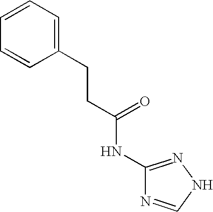 | MLS-0002068 | 860230 | 484.59 | 5.15 | 1.5 |
| 28 | 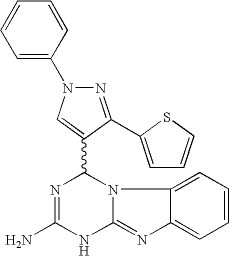 | MLS-0002876 | 855977 | 504.63 | 0.623 | 1.5 |

^{†}Activation Factor was calculated as (100-X)/100, where X is % inhibition.

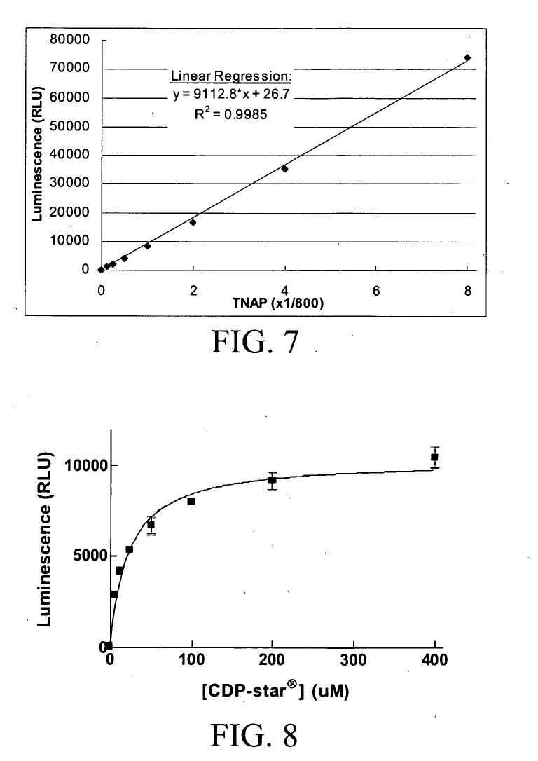

The luminescent assay was further optimized to ensure its maximum sensitivity to compounds activating TNAP. In this newly optimized assay, DEA buffer was replaced with CAPS that does not contain any alcohol phosphoacceptor. This assay can provide a more accurate measure of phosphatase activity, as opposed to transphosphorylation, activity that might be more relevant to in vivo conditions. The previously utilized assay was performed in the presence DEA at a concentration of CDP-star equal to its Kₘ value. However, the appropriate concentration of the components was needed for the new buffer. TNAP activity vs. its concentration was tested as a function of TNAP concentrations (FIG. 7).

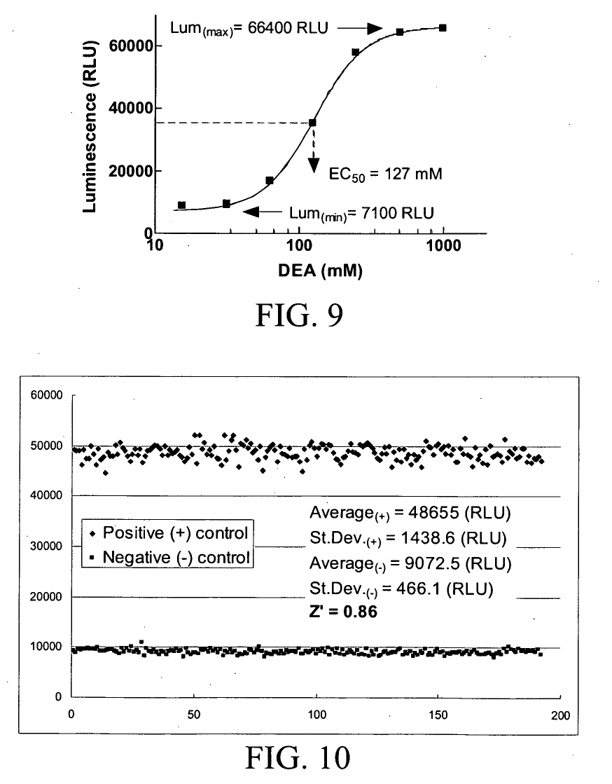

It was observed that TNAP activity was linearly dependent over an extended range of TNAP concentrations. A 1/800 concentration of TNAP was used for further work; this concentration is 1315-fold above the limit of detection of the assay. To ensure that substrate concentration is correctly adjusted in the new assay, TNAP activity was tested in the presence of varied CDP-star® concentrations (FIG. 8). It was decided to fix the concentration of CDP-star® at 25 uM (˜Kₘ) to provide enough sensitivity even for compounds competitive with the CDP-star® substrate. In the next experiment, the activation of TNAP was tested with DEA (FIG. 9).

It was observed that half-maximal activation corresponds to 127 mM DEA. Maximal activation resulted in 9.4-fold higher activity than in the absence of DEA. 600 mM DEA (pH 9.8) was chosen as the positive control for TNAP activation screening. It was previously shown that DMSO at a concentration of 2% did not have any effect on the catalytic properties of TNAP or the CDP-star-based luminescent reaction. The performance of the assay in 384-well plates was tested in the presence and absence of DEA (FIG. 10). The TNAP activation assay demonstrated good statistics (Z′=0.86) and stability over time. Both TNAP and CDP-star working solutions are stable at room temperature for several days without any loss of signal.

Since the size of MLSMR compound collection continues to grow, it would clearly be beneficial to test the expanded compound collection with an assay that was specifically optimized to enhance the possibility of identifying TNAP activators. Thus, MLSCN compounds can be screened using this newly optimized assay in search of compounds that are potent activators of TNAP. In the proposed screen, 600 mM DEA (pH 9.8) in 2% DMSO can be utilized as a positive control.

### G. References

Ali, S. Y., Sajdera, S. W. and Anderson, H. C. (1970) Isolation and characterization of calcifying matrix vesicles from epiphyseal cartilage. Proc. Natl. Acad. Sci. USA 67:1513-1520.
Anderson, H. C., Garimella, R. and Tague, S. E. (2005) The role of matrix vesicles in growth plate development and biomineralization. Frontiers in Bioscience. 10: 822-837.
Anderson, H. C., Hsu, H. H., Morris, D. C., Fedde, K. N. and Whyte, P. W. (1997) Matrix vesicle in osteomalacic hypophosphatasia bone contain apatite-like mineral crystals. Am. J. Path. 151: 1555-1561.
Anderson, H. C., Sipe, J. E., Hessle, L., Dhamayamraju, R., Atti, E., Camacho, N. P. and Millán, J. L. (2004) Impaired calcification around matrix vesicles of growth plate and bone in alkaline phosphatase-deficient mice. Am. J. Pathol. 164: 841-847.
Balsan, S., M. Garabedian, M. Larchet, A. M. Gorski, G. Coumot C. Tau, A. Bourdeau, C. Silve, and C Ricour. (1986) Long-term nocturnal calcium infusions can cure rickets and promote normal mineralization in heriditary resistance to 1, 25-Dihydroxyvitamin D. J Clin Invest 77: 1661-1667.
Bernard, G. W. (1978) Ultrastructural localization of alkaline phosphatase in initial membranous osteogenesis. Clin. Orthop. 135: 218-225.
Bollen, M., Gijsbers, R., Ceulemans, H., Stalmans, W. and Stefan, C. (2000) Nucleotide pyrophophatases-phosphodiesterases on the move. Crit. Rev. Biochem. Mol. Biol. 35: 393-432.
Boskey, A., Spevak, L., Paschalis, E., Doty, S. and McKee, M. (2002) Osteopontin deficiency increases mineral content and mineral crystallinity in mouse bone. Calcif. Tissue Int. 71: 145-154.
Bucay N. Sarosi I. Dunstan C R. Morony S. Tarpley J. Capparelli C. Scully S. Tan H L. Xu W. Lacey D L. Boyle W J. Simonet W S. (1998) osteoprotegerin-deficient mice develop early onset osteoporosis and arterial calcification. Genes & Development. 12:1260-1268.
Denhardt D T. Noda M. O'Regan A W. Pavlin D. Berman J S. (2001) Osteopontin as a means to cope with environmental insults: regulation of inflammation, tissue remodeling, and cell survival. J. Clin. Invest. 107:1055-1061.
Di Mauro, S., Manes, T., Hessle, H., Kozlenkov, A., Pizauro, J. M., Hoylaerts, M. F. and Millán, J. L. (2002) Kinetic characterization of hypophosphatasia mutations with physiological substrates. J. Bone Min. Res. 17: 1383-1391.
Dunstan C R. Boyce R. Boyce B F. Garrett I R. Izbicka E. Burgess W H. Mundy G R. (1999) Systemic administration of acidic fibroblast growth factor (FGF-1) prevents bone loss and increases new bone formation in ovariectomized rats. J. Bone Miner. Res. 14:953-959.
Erlebacher, A. and R. Derynck (1996) Increased expression of TGF-b2 in osteoblasts results in an osteoporosis-like phenotype. J. Cell Biol. 132: 195-210.
Fallon, M. D., Whyte, M. P. and Teitelbaum, S. L. (1980) Stereospecific inhibition of alkaline phosphatase by L-tetramisole prevents in vitro cartilage calcification. Lab. Invest. 43: 489-494.
Fedde, K. N., L. Blair, J. Silverstein, S. P. Coburn, L. M. Ryan, R. S. Weinstein, K. Waymire, S. Narisawa, J. L. Millán, G. R. MacGregor, and M. P. Whyte. (1999) Alkaline phosphatase knockout mice recapitulate the metabolic and skeletal defects of infantile hypophosphatasia. J. Bone Min. Res. 14: 2015-2026.
Fleisch, H., Reska, A., Rodan, G. and Rogers, M. (2002) Bisphosphonates-Mechanisms of action. In Principles of Bone Biology, Second Edition (Eds. Bilezikian, Raisz and Rodan), Academic Press, San Diego, Calif. Pages 1361-1385.
Franzén, A. and Heinegård, D. (1985) Isolation and characterization of two sialoproteins present in only bone calcified tissue. Biochem. J. 232: 715-724.
Fukushi, M., Amizuka, N., Hoshi, K., Ozawa, H., Kumagai, H., Omura, S., Misumi, Y., Ikehara, Y., and Oda, K. (1998) Intracellular retention and degradation of tissue-nonspecific alkaline phosphatase with a Gly317-->Asp substitution associated with lethal hypophosphatasia. Biochem. Biophys. Res. Comm. 246: 613-618.
Garimella R, Bi X, Camacho N, Sipe J, Anderson H C (2004): Primary culture of rat growth plate chondrocytes: an in vitro model of growth plate histotype, matrix vesicle biogenesis and mineralization. Bone 34: 961-970.
Giachelli, C. M. 2003. Vascular calcification: in vitro evidence for the role of inorganic phosphate. J Am Soc Nephrol 14: S300-S304.
Grobben, B., Claes, P., Roymans, D., Esmans, E. L., Van Onckelen, H., Slegers, H. (2000) Ecto-nucleotide pyrophosphatase modulates the purinoceptor-mediated signal transduction and is inhibited by purinoceptor antagonists. Brit J. Pharm. 130:139-145.
Hakim, F. T., Cranley, R., Brown, K. S., Eanes, E. D., Hame, L. and Oppenheim, J. J. (1984) Hereditary joint disorder in progressive ankylosis (ank/ank) mice. I. Association of calcium hydroxyapatite deposition with inflammatory arthropathy. Arthritis Rheum. 27: 1411-1420.
Harmey, D., Hessle, L., Narisawa, S., Johnson, K., Terkeltaub, R. and Millán, J. L. (2004) Concerted regulation of inorganic pyrophosphate and osteopontin by Akp2, Enpp1 and Ank. An integrated model of the pathogenesis of mineralization disorders. Am. J. Pathol. 164: 1199-1209.
Harmey, D., Kristen A. Johnson, K. A., Hoylaerts, M. F., Masaki Noda, M., Camacho, N., Terkeltaub, R and Millán, J. L. Elevated osteopontin levels contribute to the hypophosphatasia phenotype in Akp2−/− mice. J. Bone Min. Res. In Press (2006).
Hashimoto, S., Ochs, R. L., Rosen, F., Quach, J., McCabe, G., Solan, J., Seegmiller, J. E., Terkeltaub, R. and Lotz, M. (1998) Chondrocyte-derived apoptotic bodies and calcification of articular cartilage. Proc. Natl. Acad. Sci. USA 95: 3094-3099.
Heaney, R. P. (2002) Calcium. In Principles of Bone Biology, Second Edition (Eds. Bilezikian, Raisz and Rodan), Academic Press, San Diego, Calif. Pages 1325-1337.
Henthom, P. S., M. Raducha, K. N. Fedde, M. A. Lafferty, and M. P. Whyte. (1992) Different missense mutations at the tissue-nonspecific alkaline phosphatase gene locus in autosomal recessively inherited forms of mild and severe hypophosphatasia. Proc. Natl. Acad. Sci. USA 89: 9924-8.

Hessle, L., Johnsson, K. A., Anderson, H. C., Narisawa, S., Sali, A., Goding, J. W., Terkeltaub, R. and Millán, J. L. (2002) Tissue-nonspecific alkaline phosphatase and plasma cell membrane glycoprotein-1 are central antagonistic regulators of bone mineralization. Proc. Natl. Acad. Sci. USA 99: 9445-9449.
Holtz, K. M. and Kantrowitz, E. R. (1999) The mechanism of the alkaline phosphatase reaction: insights from NMR, crystallography and site-specific mutagenesis. FEBS Lett. 462: 7-11.
Hosoda N. Hoshino S I. Kanda Y. Katada T. (1999) Inhibition of phosphodiesterase/pyrophosphatase activity of PC-1 by its association with glycosaminoglycans. Eur. J. Biochem. 265:763-770.
Ho, A. M., M. D. Johnson, and D. M. Kingsley. (2000) Role of the mouse ank gene in control of tissue calcification and arthritis. Science 289: 265-70.
Hodsman, A. B., Hanley, D. A., Watson, P. H. and Fraher, L. J. (2002) Parathyroid Hormone. In Principles of Bone Biology, Second Edition (Eds. Bilezikian, Raisz and Rodan), Academic Press, San Diego, Calif. Pages 1305-1324.
Huang, R., Rosenbach, M., Vaughn, R., Provvedini, D., Rebbe, N., Hickman, S., Goding, J. and Terkeltaub, R. (1994) Expression of the murine plasma cell nucleotide pyrophosphohydrolase PC-1 is shared by human liver, bone, and cartilage cells. Regulation of PC-1 expression in osteosarcoma cells by transforming growth factor-beta. J. Clin. Invest. 94: 560-567.
Hunter, G. K., Kyle, C. L. and Goldberg, H. A. (1994) Modulation of crystal formation by bone phosphoproteins; structural specificity of the osteopontin-mediated inhibition of hydroxyapatite formation. Biochem. J. 300: 723-728.
Ihara, H., Denhardt, D. T., Furuya, K., Yamashita, T., Muguruma, Y., Tsuji, K., Hruska, K. A., Higashio, K., Enomoto, S., Nifuju, A., Rittling, S. R. and Noda, M. (2001) Parathyroid hormone-induced bone resorption does not occur in the absence of osteopontin. J. Biol. Chem. 276: 13065-13071.
Johnson, K., Moffa, A., Chen, Y., Pritzker, K., Goding, J. and Terkeltaub, R. (1999) Matrix vesicle plasma membrane glycoprotein-1 regulates mineralization by murine osteoblastic MC3T3 cells. J. Bone Min. Res 14: 883-892.
Johnson, K. A., Hessle, L., Wennberg, C., Mauro, S., Narisawa, S., Goding, J., Sano, K., Millán, J. L. and Terkeltaub, R. (2000) Tissue-nonspecific alkaline phosphatase (TNAP) and plasma cell membrane glycoprotein-1 (PC-1) act as selective and mutual antagonists of mineralizing activity by murine osteoblasts. Am. J. Phys. Regulatory and Integrative Physiology 279: R1365-1377.
Johnson, K., Hashimoto, S., Lotz, M., Pritzker, K., Goding, J. and Terkeltaub, R. (2001) Focal expression of plasma cell membrane glycoprotein-1 (PC-1) is both a marker and potential direct promotor of calcification in knee meniscal chondrocalcinosis. Arthr. Rheum. 44, 1071-1081
Johnson, K., Goding, J., Van Etten, D., Sali, A., Hu, S-I, Farley, D., Krug, H., Hessle, L., Millán, J. L., and Terkeltaub, R. (2003) Linked deficiencies in extracellular inorganic pyrophosphate (PPi) and osteopontin expression mediate pathologic ossification in PC-1 null mice. J. Bone Min. Res. 18: 994-1004.
Johnson K. Polewski M. van Etten D. Terkeltaub R. (2005) Chondrogenesis mediated by PPi depletion promotes spontaneous aortic calcification in NPP1−/− mice. Arteriosclerosis, Thrombosis & Vascular Biology. 25:686-691.
Jones, A. C., Chucjk, A. J., Arie, E. A., Green, D. J. and Doherty, M. (1992) Diseases associated with calcium pyrophosphate deposition disease. Semin. Arthritis Rheum. 22:188-202.
Keykhosravani M. Doherty-Kirby A. Zhang C. Brewer D. Goldberg H A. Hunter G K. Lajoie G. (2003) Comprehensive identification of post-translational modifications of rat bone osteopontin by mass spectrometry. Biochemistry. 44: 6990-7003.
Kimmel, D. B. (2002) Animal models of osteoporosis research. In Principles of Bone Biology, Second Edition (Eds. Bilezikian, Raisz and Rodan), Academic Press, San Diego, Calif. Pages 1635-1655.
Kozlenkov, A., Manes, T., Hoylaerts, M. F. and Millán, J. L. (2002) Function assignment to conserved residues in mammalian alkaline phosphatases. J. Biol. Chem. 277:22992-22999.
Kozlenkov, A., Hoylaerts, M. F., Ny, T., Le Du, M-H. And Millán, J. L. (2004) Residues determining the binding specificity of uncompetitive inhibitors to tissue-nonspecific alkaline phosphatase. J. Bone Min Res. 19: 1862-1872.
Lai C. F., Chaudhary L., Fausto A., Halstead L. R., Ory D. S., Avioli L. V. and Cheng S. L. (2001) Erk is essential for growth, differentiation, integrin expression, and cell function in human osteoblastic cells. J Biol. Chem. 276:14443-50.
Lai C. F. and Cheng S. L. (2005) Alphavbeta integrins play an essential role in BMP-2 induction of osteoblast differentiation. J Bone Miner Res. 20:330-40.
Lazdunski, M., Petitclerc, C., Chappelet, D. and Lazdunski, C. (1971) Flip-Flop mechanism in Enzymology. A model: the alkaline phosphatase of Escherichia Coli. Eur. J. Biochem. 20, 124-139.
Le Du, M. H., T. Stigbrand, M. J. Taussig, A. Menez and E. A. Stura. (2001) Crystal structure of alkaline phosphatase from human placenta at 1.8 A resolution. Implication for a substrate specificity. J. Biol. Chem. 276: 9158-9165.
Le Du, M-H and Millán, J. L. (2002) Structural evidence of functional divergence in human alkaline phosphatases. J. Biol. Chem. 277: 49808-49814.
Lewis, D. B., H. D. Liggitt, E. L. Effmann, S. T. Motley, S. L. Teitelbaum, K. J. Jepsen, S. A. Goldstein, J. Bonadio, J. Carpenter, and R. M. Perlmutter (1993) Osteoporosis induced in mice by overproduction of interleukin 4. Proc. Natl. Acad. Sci. 90: 11618-11622.
Llinas, P., Stura, E. A., Ménez, A., Kiss, Z., Stigbrand, T., Millán, J. L. and Le Du, M-H. (2005) Structural studies of human placental alkaline phosphatase in complex with functional ligands. J. Mol. Biol. 350, 441-451.
Liaw, L., Birk, D. E., Ballas, C. B., Whitsitt, J. S., Davidson, J. M. and Hogan, B. L. (1998) Altered wound healing in mice lacking a functional osteopontin gene (spp1). J. Clin. Invest. 101:1468-1478.
Majeska, R. J. and Wuthier, R. E. (1975) Studies on matrix vesicles isolated from chick epiphyseal cartilage. Association of pyrophosphatase and ATPase activities with alkaline phosphatase. Biochim. Biophys. Acta 391: 51-60.
Masuda, I., Iyama, K., Halligan, B. D., Barbieri, J. T., Haas, A. L., McCarty, D. J. and Ryan, L. M. (2001) Variations in site and levels of expression of chondrocyte nucleotide pyrophosphohydrolase with aging. J. Bone Min. Res. 16: 868-875.
Meyer, J. L. (1984) Studies on matrix vesicles isolated from chick epiphyseal cartilage. Association of pyrophosphatase and ATPase activities with alkaline phosphatase. Arch. Biochem. Biophys. 231: 1-8.
McHugh K. P., Hodivala-Dilke K., Zheng M. H., Namba N., Lam J., Novack D., Feng X., Ross F. P., Hynes R. O., and Teitelbaum S. L. (2000) Mice lacking beta3 integrins are osteosclerotic because of dysfunctional osteoclasts. J. Clin. Invest. 105: 433-440.
Millán, J. L. Mammalian alkaline phosphatases. From biology to applications in medicine and biotechnology. Wiley-VCH Verlag GmbH & Co, Weinheim, Germany (2006). pgs. 1-322.
Mizuno A. Amizuka N. Irie K. Murakami A. Fujise N. Kanno T. Sato Y. Nakagawa N. Yasuda H. Mochizuki S. Gomibuchi T. Yano K. Shima N. Washida N. Tsuda E. Morinaga T. Higashio K. Ozawa H. (1998) Severe osteoporosis in mice lacking osteoclastogenesis inhibitory factor/osteoprotegerin. Biochem. Biophys. Res. Comm. 247:610-615.
Morris, D. C., Masuhara, K., Takaoka, K., Ono, K. and Anderson, H. C. (1992) Immunolocalization of alkaline phosphatase in osteoblasts and matrix vesicles of human fetal bone. Bone Min. 19: 287-298.
Moss, D. W., Eaton, R. H., Smith, J. K. and Whitby, L. G. (1967) Association of inorganic pyrophosphatase activity with human alkaline phosphatase preparations. Biochem. J. 102: 53-57.
Murshed, M., Harmey, D., Millán, J. L., McKee, M. D. and Karsenty, G. (2005) Broadly expressed genes accounts for the special restriction of ECM mineralization to bone. Genes Dev. 19: 1093-1104.
Narisawa, S., Fröhlander, N. and Millán, J. L. (1997) Inactivation of two mouse alkaline phosphatase genes and establishment of a model of infantile hypophosphatasia. Dev. Dyn. 208: 432-446.
Nürnberg, P., Theile, H., Chandler, D., Höhne, W., Cunningham, M. L., and Ritter H, (2001) Heterozygous mutations in ANKH, the human ortholog of the mouse progressive ankylosis gene, result in craniometaphyseal dysplasia. Nat. Genet. 28: 37-41.
Okawa, A., I. Nakamura, S. Goto, H. Moriya, Y. Nakamura, and S. Ikegawa. (1998) Mutation in Npps in a mouse model of ossification of the posterior longitudinal ligament of the spine. Nat. Genet. 19: 271-3.
Oldberg, A., Franzén, A., Heinegård, D. (1986) Cloning and sequence analysis of rat bone sialoprotein (osteopontin) cDNA reveals an arg-gly-asp cell-binding sequence. Proc. Natl. Acad. Sci. USA 83: 8819-8823.
Reeve J. (2002) Recombinant human parathyroid hormone. BMJ. 324:435-436.
Rezende, A., Pizauro, J., Ciancaglini, P. and Leone, F. (1994) Phosphodiesterase activity is a novel property of alkaline phosphatase from osseous plate. Biochem. J. 301: 517-522.

Rittling, S. R., Matsumoto, H. N., McKee, M. D., Nanci, A., An, X. R. and Novick, K. E. (1998) Mice lacking osteopontin show normal development and bone structure bur display altered osteoclast formation in vitro. J. Bone Min. Res. 13: 1101-1111.
Roberts, S., Narisawa, S., Harmey, D., Millán, J. L. and Farquharson, C. (2007) Functional involvement of PHOSPHO1 in matrix vesicle-mediated skeletal calcification. J. Bone Min. Res. In press.
Robison, R. (1923) The possible significance of hexosephosphoric esters in ossification. Biochem. J. 17: 286-293.
Rodan, G. A. Raisz, L. G., and Bilezikian, J. P. (2002) Pathophysiology of osteoporosis. In Principles of Bone Biology, Second Edition (Eds. Bilezikian, Raisz and Rodan), Academic Press, San Diego, Calif. Pages 1275-1289.
Ruf N. Uhlenberg B. Terkeltaub R. Nurnberg P. Rutsch F. (2005) The mutational spectrum of ENPP1 as arising after the analysis of 23 unrelated patients with generalized arterial calcification of infancy (GACI). Human Mutation. 25:98.
Rutsch, F. Ruf, N. Vaingankar, S. Toliat, M. R. Suk, A. Hohne, W., Schauer, G., Lehmann, M. Roscioli, T. Schnabel, D., Epplen, J. T., Knisely, A., Superti-Furga, A., McGill, J., Filippone, M., Sinaiko, A. R., Vallance, H., Hinrichs, B., Smith, W., Ferre, M., Terkeltaub, R., Nurnberg, P. (2003) Mutations in ENPP1 are associated with ‘idiopathic’ infantile arterial calcification. Nature Genetics 34:379-381.
Ruoslahti E. and Pirschbacher M. P. (1987) New perspectives in cell adhesion: RGD and integrins. Science. 238:491-497.
Sali, A., J. M. Favaloro, R. Terkeltaub, and J. W. Goding. (1999) Germline deletion of the nucleoside triphosphate pyrophosphohydrolase (NTPPPH) plasma cell membrane glycoprotein (PC-1) produces abnormal calcification of periarticular tissues. Maastricht, Shaker Publishing B V.
Sampson, H. W. and Davis, J. S. (1988) Histopathology of the intervertebral disc of progressive ankylosis mice. Spine 13: 650-654.
Sampson, H. W. and Davis, J. S. (1988) Histopathology of the intervertebral disc of progressive ankylosis mice. Spine 13: 650-654.
Shibata, H., Fukushi, M., Igarashi, A., Misumi, Y., Ikehara, Y., Ohashi, Y., and Oda, K. (1988) Defective intracellular transport of tissue-nonspecific alkaline phosphatase with an Ala162-->Thr mutation associated with lethal hypophosphatasia. J. Biochem. 123: 968-977.
Sheppard, D. (2000). In vivo functions of integrins: lessons from null mutations in mice. Matrix Biol 19: 203-209.
Sherman S. (2001) Preventing and treating osteoporosis: strategies at the millennium. Annals of the New York Academy of Sciences. 949:188-197.
Shimogori, H., and Yamashita, H. (2001) Rapid correction of vestibular imbalance by intraccochlear administration of ATP in a guinea pig model of unilateral peripheral vestibular disorder. Neuroscience Letters. 315: 69-72.
Sodek J, Zhu B, Huynh M H, Brown T J, Ringuette M (2002) Novel functions of the matricellular proteins osteopontin and osteonectin/SPARC. Connective Tissue Res. 2 43:308-319.
Steitz S A. Speer M Y. McKee M D. Liaw L. Almeida M. Yang H. Giachelli C M. (2002) Osteopontin inhibits mineral deposition and promotes regression of ectopic calcification. Am. J. Pathol. 161:2035-2046.
Sweet, H. O. and Green, M. C. (1981) Progressive ankylosis, a new skeletal mutation in the mouse. J. Hered. 72: 87-93.
Takahashi, T., T. Wada, M. Mori, Y. Kokai, and S. Ishii (1996) Overexpression of the granulocyte colony stimulating factor gene leads to osteoporosis in mice. Lab. Invest. 74: 827-834.
Terkeltaub R, Rosenbach M, Fong F, Goding J (1994) Causal link between nucleotide pyrophosphohydrolase overactivity and increased intracellular inorganic pyrophosphate generation demonstrated by transfection of cultured fibroblasts and osteoblasts with plasma cell membrane glycoprotein-1. Arthr. Reumat. 37: 934-941.
Terkeltaub, R. A. (2001) Inorganic pyrophosphate generation and disposition in pathophysiology. Am. J. Physiol. Cell Physiol. 281: C1-C11.
Wada, T., McKee, M. D., Steitz, S. and Giachelli, C. M. (1999) Calcification of vascular smooth muscle cell cultures; inhibition of osteopontin. Circ. Res. 84: 166-178.
Wada T. Nakashima T. Hiroshi N. Penninger J M. RANKL-RANK signaling in osteoclastogenesis and bone disease. Trends in Molecular Medicine. 12:17-25.
Wang P S. Solomon D H. Mogun H. Avorn J. (2000) HMG-CoA reductase inhibitors and the risk of hip fractures in elderly patients. [see comment]. JAMA. 283(24):3211-3216.
Wang, W., Xu, J., Du, B. and Kirsch, T. (2005) Role of the Progressive Ankylosis Gene (ank) in Cartilage Mineralization. Mol. Cell. Biol. 25: 312-323.
Weber, G. F. and Cantor, H. (1996) The immunology of Eta-1/osteopontin. Cytokine Growth Factor Rev 7: 241-248.
Weninger, M., Stinson, R. A., Plenk, H., Jr., Bock, P. & Pollak, A. Biochemical and morphological effects of human hepatic alkaline phosphatase in a neonate with hypophosphatasia. Acta Paediatr Scand Suppl 360, 154-60 (1989).
Wennberg, C., Hessle, L., Lundberg, P., Mauro, S., Narisawa, S., Lerner, U. H., and Millán, J. L. (2000) Functional characterization of osteoblasts and osteoclasts from alkaline phosphatase knockout mice. J. Bone Min. Res. 15: 1879-1888.
Whyte, M. P. (1994) Hypophosphatasia and the role of alkaline phosphatase in skeletal mineralization. Endocr. Rev. 15: 439-461.
Whyte, M. P. Hypophosphatasia. In: C. R. Scriver, A. L. Beaudet, W. S. Sly, D. Valle (eds) The metabolic and molecular bases of inherited disease, 7th ed. McGraw-Hill New York, pp 4095-4112, 1995.
Whyte, M. P. et al. Enzyme replacement therapy for infantile hypophosphatasia attempted by intravenous infusions of alkaline phosphatase-rich Paget plasma: results in three additional patients. J Pediatr 105, 926-33 (1984).
Whyte, M. P., Valdes, R., Jr., Ryan, L. M. & McAlister, W. H. Infantile hypophosphatasia: enzyme replacement therapy by intravenous infusion of alkaline phosphatase-rich plasma from patients with Paget bone disease. J Pediatr 101, 379-86 (1982).
Whyte, M. P. et al. Failure of hyperphosphatasemia by intravenous infusion of purified placental alkaline phosphatase (ALP) to correct severe hypophosphatasia: evidence against a role for circulating ALP in skeletal mineralization. J. Bone Min Res 7 (Suppl 1), S155 (1992).
Whyte, M. P. et al. Marrow cell transplantation for infantile hypophosphatasia. J Bone Miner Res 18, 624-36 (2003).
Whyte, M. P., Landt, M., Ryan, L. M., Mulivor, R. A., Henthorn, P. S., Fedde, K. N., Mahuren, J. D., and Coburn, S. P. (1995). Alkaline phosphatase: placental and tissue-nonspecific isoenzymes hydrolyze phosphoethanolamine, inorganic pyrophosphate, and pyridoxal 5′-phosphate. Substrate accumulation in carriers of hypophosphatasia corrects during pregnancy. J. Clin. Invest. 95, 1440-1445.
Yoshitake, H., Rittling, S. R., Denhardt, D. T. and Noda, M. (1999) Osteopontin-deficient mice are resistant to ovariectomy-induced bone resorption. Proc. Natl. Acad. Sci. USA 96: 8156-8160.
Zhao H., Ross F. P., and Teitelbaum S. L. (2005) Unoccupied alpha(v) beta3 integrin regulates osteoclast apoptosis by transmitting a positive death signal. Mol Endocrinol. 19:771-80.
Zurutuza, L., Muller, F., Gibrat, J. F., Taillandier, A., Simon-Bouy, B., Serre, J. L. and Mornet, E. (1999) Correlations of genotype and phenotype in hypophosphatasia. Hum. Mol. Genet. 8: 1039-1046.

1. A method of promoting bone mineral deposition in a subject, comprising administering to the subject a tissue-nonspecific alkaline phosphatase (TNAP) activator.

2. The method of claim 1, wherein the subject is in need of increased bone mineral density (BMD).

3. The method of claim 2, wherein the subject has been diagnosed with hypophosphatasia.

4. The method of claim 2, wherein the subject has been diagnosed with osteoporosis.

5. The method of claim 2, wherein the subject has been diagnosed with calcium pyrophosphate deposition disease (CPPD/chodrocalcinosis).

6. (canceled)

7. (canceled)

8. (canceled)

9. The method of claim 1, further comprising administering to the subject a TNAP peptide.

10. A method of enhancing the pyrophosphatase activity of tissue-nonspecific alkaline phosphatase (TNAP), comprising contacting the TNAP with a TNAP activator.

11. The method of claim 1, wherein the TNAP activator is a small molecule.

12. The method of claim 1, wherein the TNAP activator facilitates the release of inorganic pyrophosphate (PPᵢ) from the active site, thereby increasing the effective rate of PPᵢ hydrolysis.

13. The method of claim 1, wherein the TNAP activator is a compound having the formula: 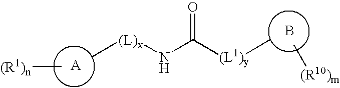
wherein A is a 5-member heterocyclic or heteroaryl ring that can optionally have from 1 to 4 hydrogen atoms substituted by an organic radical, R¹;
B represents a phenyl, cyclopentyl, cyclohexyl, or a 5-member heterocyclic ring can optionally have from 1 to 5 hydrogen atoms substituted by an organic radical, R¹⁰;
L and L¹ are each independently a linking unit having in the chain from 1 to 6 carbon atoms or from 1 to 5 carbon atoms together with from 1 to 4 heteroatoms chosen from nitrogen, oxygen, or sulfur;
the index m is from 1 to 5;
the index n from 1 to 4;
the index x is 0 or 1; and
the index y is 0 or 1.

14. The method of claim 13, wherein A is a 5-member heteroaryl ring chosen from:

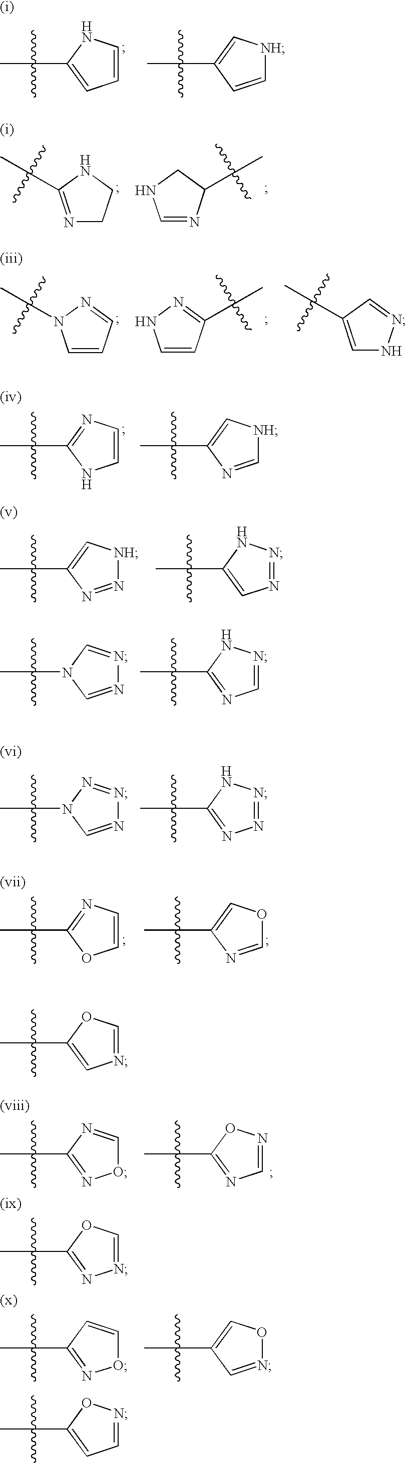

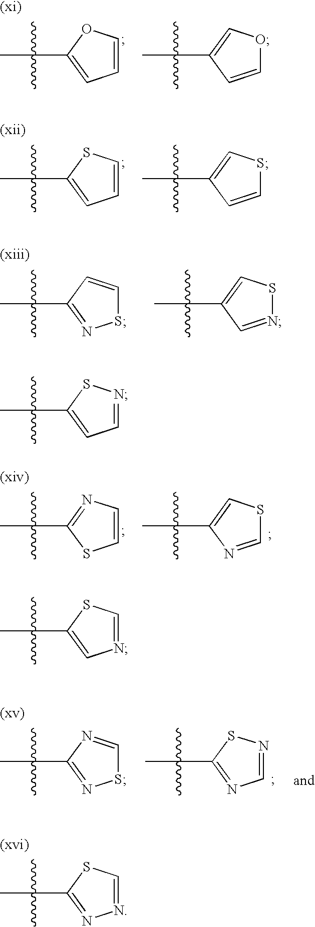

15. The method of claim 13, wherein A is a 5-member heteroaryl ring chosen from:

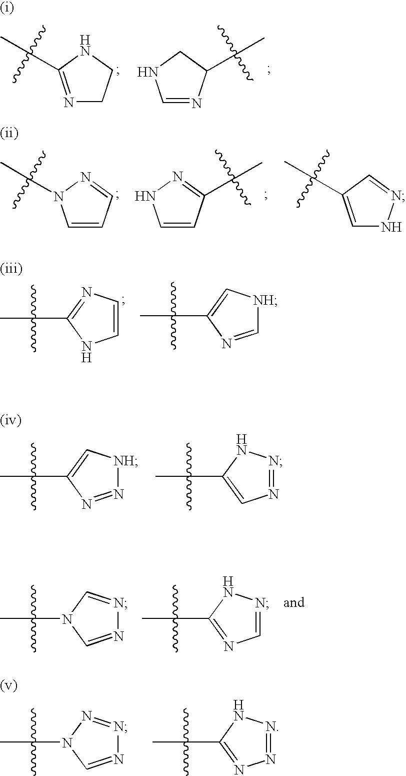

16. The method of claim 13, wherein A is 1,2,4-triazoles having the formula:

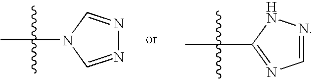

17. The method of claim 13, wherein A is unsubstituted 1,2,4-triazol-3-yl.

18. The method of claim 13, wherein each R¹ is independently:
(i) linear, branched, or cyclic alkyl, alkenyl, and alkynyl; for example, methyl, ethyl, n-propyl, iso-propyl, cyclopropyl, propylen-2-yl, propargyl, n-butyl, iso-butyl, sec-butyl, tert-butyl, cyclobutyl, n-pentyl, cyclopentyl, n-hexyl, and cyclohexyl;
(ii) substituted or unsubstituted aryl;
(iii) substituted or unsubstituted heterocyclic;
(iv) substituted or unsubstituted heteroaryl;
(V) —(CR^{3a}R^{3b})_{q}OR²;
(vi) —(CR^{3a}R^{3b})_{q}C(O)R²;
(vii) —(CR^{3a}R^{3b})_{q}C(O)OR²;
(viii) —(CR^{3a}R^{3b})_{q}C(O)N(R²)₂;
(ix) —(CR^{3a}R^{3b})_{q}OC(O)N(R²)₂;
(x) —(CR^{3a}R^{3b})_{q}N(R²)₂;
(xi) halogen;
(xii) —CHₘXₙ;
(xiii) —(CR^{3a}R^{3b})_{q}CN;
(xiv) —(CR^{3a}R^{3b})_{q}NO₂;
(xv) —(CR^{3a}R^{3b})_{q}SO₂R²; and
(xvi) —(CR^{3a}R^{3b})_{q}SO₃R²;
wherein each R² is independently hydrogen, substituted or unsubstituted C₁-C₄ linear, branched, or cyclic alkyl; or two R² units can be taken together to form a ring comprising 3-7 atoms; R^{3a} and R^{3b} are each independently hydrogen or C₁-C₄ linear or branched alkyl; the index q is from 0 to 4.

19. The method of claim 18, wherein each R¹ is independently chosen from C₁-C₄alkyl, alkenyl, or alkynyl.

20. The method of claim 19, wherein each R¹ is independently chosen from methyl, ethyl, n-propyl, iso-propyl, cyclopropyl, propylen-2-yl, propargyl, n-butyl, iso-butyl, sec-butyl, tert-butyl, or cyclobutyl.

21. The method of claim 13, wherein R¹ is chosen from 2-fluorophenyl, 2-chlorophenyl, 2-methylphenyl, 2-methoxy-phenyl, 3-fluorophenyl, 3-chlorophenyl, 3-methylphenyl, 3-methoxyphenyl, 4-fluorophenyl, 4-chlorophenyl, 4-methylphenyl, and 4-methoxyphenyl.

22. The method of claim 13, wherein R¹ is C₁-C₁₂ linear, branched, or cyclic alkyl, alkenyl; phenyl; C₁-C₉heterocyclic; or C₁-C₉heteroaryl further substituted by one or more organic radicals independently chosen from
(i) linear, branched, or cyclic alkyl, alkenyl, and alkynyl; for example, methyl (C₁), ethyl (C₂), n-propyl (C₃), iso-propyl (C₃), cyclopropyl (C₃), propylen-2-yl (C₃), propargyl (C₃), n-butyl (C₄), iso-butyl (C₄), sec-butyl (C₄), tert-butyl (C₄), cyclobutyl (C₄), n-pentyl (C₅), cyclopentyl (C₅), n-hexyl (C₆), and cyclohexyl (C₆);
(ii) —(CR^{5a}R^{5b})_{q}OR⁴; for example, —OH, —CH₂OH, —OCH₃, —CH₂OCH₃, —OCH₂CH₃, —CH₂OCH₂CH₃, —OCH₂CH₂CH₃, and —CH₂OCH₂CH₂CH₃;
(iii) —(CR^{5a}R^{5b})_{q}C(O)R⁴; for example, —COCH₃, —CH₂COCH₃, —OCH₂CH₃, —CH₂COCH₂CH₃, —COCH₂CH₂CH₃, and —CH₂COCH₂CH₂CH₃;
(iv) —(CR^{5a}R^{5b})_{q}C(O)OR⁴; for example, —CO₂CH₃, —CH₂CO₂CH₃, —CO₂CH₂CH₃, —CH₂CO₂CH₂CH₃, —CO₂CH₂CH₂CH₃, and —CH₂CO₂CH₂CH₂CH₃;
(v) —(CR^{5a}R^{5b})_{q}C(O)N(R⁴)₂; for example, —CONH₂, —CH₂CONH₂, —CONHCH₃, —CH₂CONHCH₃, —CON(CH₃)₂, and —CH₂CON(CH₃)₂;
(vi) —(CR^{5a}R^{5b})_{q}OC(O)N(R⁴)₂; for example, —OC(O)NH₂, —CH₂OC(O)NH₂, —OC(O)NHCH₃, —CH₂OC(O)NHCH₃, —OC(O)N(CH₃)₂, and —CH₂OC(O)N(CH₃)₂;
(vii) —(CR^{5a}R^{5b})_{q}N(R⁴)₂; for example, —NH₂, —CH₂NH₂, —NHCH₃, —N(CH₃)₂, —NH(CH₂CH₃), —CH₂NHCH₃, —CH₂N(CH₃)₂, and —CH₂NH(CH₂CH₃);
(viii) halogen: —F, —Cl, —Br, and —I;
(ix) —CHₘXₙ; wherein X is halogen, m is from 0 to 2, m+n=3; for example, —CH₂F, —CHF₂, —CF₃, —CCl₃, or —CBr₃;
(x) —(CR^{5a}R^{5b})_{q}CN; for example; —CN, —CH₂CN, and —CH₂CH₂CN;
(xi) —(CR^{5a}R^{5b})_{q}NO₂; for example; —NO₂, —CH₂NO₂, and —CH₂CH₂NO₂;
(xii) —(CR^{5a}R^{5b})_{q}SO₂R⁴; for example, —SO₂H, —CH₂SO₂H, —SO₂CH₃, —CH₂SO₂CH₃, —SO₂C₆H₅, and —CH₂SO₂C₆H₅; and
(xiii) —(CR^{5a}R^{5b})_{q}SO₃R⁴; for example, —SO₃H, —CH₂SO₃H, —SO₃CH₃, —CH₂SO₃CH₃, —SO₃C₆H₅, and —CH₂SO₃C₆H₅;
wherein each R⁴ is independently hydrogen, substituted or unsubstituted C₁-C₄ linear, branched, or cyclic alkyl; or two R⁴ units can be taken together to form a ring comprising 3-7 atoms; R^{5a} and R^{5b} are each independently hydrogen or C₁-C₄ linear or branched alkyl; the index p is from 0 to 4.

23. The method of claim 13, wherein the A ring is a 1,2,4-triazole ring substituted by at least one organic radical chosen from 2-fluorophenyl, 2-chlorophenyl, 2-methylphenyl, 2-methoxy-phenyl, 3-fluorophenyl, 3-chlorophenyl, 3-methylphenyl, 3-methoxyphenyl, 4-fluorophenyl, 4-chlorophenyl, 4-methylphenyl, and 4-methoxyphenyl.

24. The method according to claim 13, wherein B is phenyl or substituted phenyl.

25. The method according to claim 13, wherein B is substituted by from 1 to 5 organic radicals, R¹⁰, each of which are independently chosen from:
(i) linear, branched, or cyclic alkyl, alkenyl, and alkynyl;
(ii) substituted or unsubstituted aryl;
(iii) substituted or unsubstituted heterocyclic;
(iv) substituted or unsubstituted heteroaryl;
(v) —(CR^{12a}R^{12b})_{q}OR¹¹;
(vi) —(CR^{12a}R^{12b})_{q}C(O)R¹¹;
(vii) —(CR^{12a}R^{12b})_{q}C(O)OR¹¹;
(viii) —(CR^{12a}R^{12b})_{q}C(O)N(R¹¹)₂;
(ix) —(CR^{12a}R^{12b})_{q}OC(O)N(R¹¹)₂;
(x) —(CR^{12a}R^{12b})_{q}N(R¹¹)₂;
(xi) halogen;
(xii) —CHₘXₙ; wherein X is halogen, m is from 0 to 2, m+n=3;
(xiii) —(CR^{12a}R^{12b})_{q}CN;
(xiv) —(CR^{12a}R^{12b})_{q}NO₂;
(xv) —(CR^{12a}R^{12b})_{q}SO₂R¹¹; and
(xvi) —(CR^{12a}R^{12b})_{q}SO₃R¹¹;
wherein each R¹¹ is independently hydrogen, substituted or unsubstituted C₁-C₄ linear, branched, or cyclic alkyl; or two R¹¹ units can be taken together to form a ring comprising 3-7 atoms; R^{12a} and R^{12b} are each independently hydrogen or C₁-C₄ linear or branched alkyl; the index q is from 0 to 4.

26. The method according to claim 25, wherein R¹⁰ is further substituted by one or more organic radicals independently chosen from:
(i) linear, branched, or cyclic alkyl, alkenyl, and alkynyl;
(ii) —(CR^{14a}R^{14b})_{q}OR¹³;
(iii) —(CR^{14a}R^{14b})_{q}C(O)R¹³;
(iv) —(CR^{14a}R^{14b})_{q}C(O)OR
(v) —(CR^{14a}R^{14b})_{q}C(O)N(R¹³)₂;
(vi) —(CR^{14a}R^{14b})_{q}OC(O)N(R¹³)₂;
(vii) —(CR^{14a}R^{14b})_{q}N(R¹³)₂;
(viii) halogen;
(ix) —CHₘXₙ; wherein X is halogen, m is from 0 to 2, m+n=3;
(x) —(CR^{14a}R^{14b})_{q}CN;
(xi) —(CR^{14a}R^{14b})_{q}NO₂;
(xii) —(CR^{14a}R^{14b})_{q}SO₂R¹³; and
(xiii) —(CR^{14a}R^{14b})_{q}SO₃R¹³;
wherein each R¹³ is independently hydrogen, substituted or unsubstituted C₁-C₄ linear, branched, or cyclic alkyl; or two R¹³ units can be taken together to form a ring comprising 3-7 atoms; R^{14a} and R^{14b} are each independently hydrogen or C₁-C₄ linear or branched alkyl; the index p is from 0 to 4.

27. The method according to claim 13, wherein B is a phenyl ring substituted with from 1 to 5 organic radicals chosen from:
(i) methyl, ethyl, n-propyl, iso-propyl, cyclopropyl, or tert-butyl;
(ii) —OH, —CH₂OH, —OCH₃, —CH₂OCH₃, or —OCH₂CH₃;
(iii) —COCH₃;
(iv) —CO₂CH₃, —CH₂CO₂CH₃, or —CO₂CH₂CH₃;
(v) —CONH₂, —CONHCH₃, or —CON(CH₃)₂;
(vi) —NH₂, —NHCH₃, or —N(CH₃)₂;
(vii) —F, —Cl, —Br, and —I;
(viii) —CF₃;
(ix) —CN;
(x) —NO₂; and
(xi) —SO₂CH₃ or —SO₂C₆H₅.

28. The method according to claim 13, wherein B is a substituted phenyl ring chosen from 2-fluorophenyl, 3-fluorophenyl, 4-fluorophenyl, 2,3-difluoro-phenyl, 2,4-difluorophenyl, 2,5-difluorophenyl, 2,6-difluorophenyl, 3,4-difluorophenyl, 3,5-difluorophenyl, 2,3,4-trifluorophenyl, 2,3,5-trifluorophenyl, 2,3,6-trifluorophenyl, 2,4,6-trifluorophenyl, 2,3,4,5-tetrafluorophenyl, 2,3,4,6-tetrafluorophenyl, 2,3,4,5,6-pentafluorophenyl, 2-chlorophenyl, 3-chlorophenyl, 4-chlorophenyl, 2,3-dichloro-phenyl, 2,4-dichlorophenyl, 2,5-dichlorophenyl, 2,6-dichlorophenyl, 3,4-dichlorophenyl, 3,5-dichlorophenyl, 2,3,4-trichloro-phenyl, 2,3,5-trichlorophenyl, 2,3,6-trichlorophenyl, 2,4,6-trichlorophenyl, 2,3,4,5-tetrachlorophenyl, 2,3,4,6-tetrachlorophenyl, 2,3,4,5,6-pentachloro-phenyl, 2-bromophenyl, 3-bromophenyl, 4-bromophenyl, 2,3-dibromophenyl, 2,4-dibromophenyl, 2,5-dibromophenyl, 2,6-dibromophenyl, 3,4-dibromo-phenyl, 3,5-dibromophenyl, 2,3,4-tribromophenyl, 2,3,5-tribromophenyl, 2,3,6-tribromophenyl, 2,4,6-tribromophenyl, 2,3,4,5-tetrabromophenyl, 2,3,4,6-tetrabromophenyl, 2,3,4,5,6-pentabromophenyl, 2-hydroxyphenyl, 3-hydroxy-phenyl, 4-hydroxyphenyl, 2,3-dihydroxyphenyl, 2,4-dihydroxyphenyl, 2,5-dihydroxyphenyl, 2,6-dihydroxyphenyl, 3,4-dihydroxyphenyl, 3,5-dihydroxy-phenyl, 2,3,4-trihydroxyphenyl, 2,3,5-trihydroxyphenyl, 2,3,6-trihydroxy-phenyl, 2,4,6-trihydroxyphenyl, 2,3,4,5-tetrahydroxyphenyl, 2,3,4,6-tetra-hydroxyphenyl, 2,3,4,5,6-pentahydroxyphenyl, 2-methoxyphenyl, 3-methoxy-phenyl, 4-methoxyphenyl, 2,3-dimethoxyphenyl, 2,4-dimethoxyphenyl, 2,5-dimethoxyphenyl, 2,6-dimethoxyphenyl, 3,4-dimethoxyphenyl, 3,5-dimethoxy-phenyl, 2,3,4-trimethoxyphenyl, 2,3,5-trimethoxyphenyl, 2,3,6-trimethoxy-phenyl, 2,4,6-trimethoxyphenyl, 2,3,4,5-tetramethoxyphenyl, 2,3,4,6-tetra-methoxyphenyl, 2,3,4,5,6-pentamethoxyphenyl, 2-ethoxyphenyl, 3-ethoxy-phenyl, 4-ethoxyphenyl, 2,3-diethoxyphenyl, 2,4-diethoxyphenyl, 2,5-diethoxyphenyl, 2,6-diethoxyphenyl, 3,4-diethoxyphenyl, 3,5-diethoxyphenyl, 2,3,4-triethoxyphenyl, 2,3,5-triethoxyphenyl, 2,3,6-triethoxyphenyl, 2,4,6-triethoxyphenyl, 2,3,4,5-tetraethoxy-phenyl, 2,3,4,6-tetraethoxyphenyl, 2,3,4,5,6-pentaethoxyphenyl, 2-methylphenyl, 3-methylphenyl, 4-methylphenyl, 2,3-dimethylphenyl, 2,4-dimethylphenyl, 2,5-dimethyl-phenyl, 2,6-dimethyl-phenyl, 3,4-dimethylphenyl, 3,5-dimethylphenyl, 2,3,4-trimethyl-phenyl, 2,3,5-trimethylphenyl, 2,3,6-trimethylphenyl, 2,4,6-trimethylphenyl, 2,3,4,5-tetra-methylphenyl, 2,3,4,6-tetramethylphenyl, 2,3,4,5,6-pentamethylphenyl, 2-ethylphenyl, 3-ethylphenyl, 4-ethylphenyl, 2,3-diethylphenyl, 2,4-diethyl-phenyl, 2,5-diethylphenyl, 2,6-diethylphenyl, 3,4-diethylphenyl, 3,5-diethyl-lphenyl, 2,3,4-triethyl-phenyl, 2,3,5-triethylphenyl, 2,3,6-triethylphenyl, 2,4,6-triethylphenyl, 2,3,4,5-tetraethylphenyl, 2,3,4,6-tetraethylphenyl, 2,3,4,5,6-pentaethylphenyl, 2-(trifluoro-methyl)phenyl, 3-(trifluoromethyl)phenyl, 4-(trifluoromethyl)phenyl, 2,3-di(trifluoro-methyl)phenyl, 2,4-di(trifluoromethyl)-phenyl, 2,5-di(trifluoromethyl)phenyl, 2,6-di(trifluoromethyl)phenyl, 3,4-di(trifluoromethyl)phenyl, 3,5-di(trifluoromethyl)phenyl, 2,3,4-tri(trifluoro-methyl)phenyl, 2,3,5-tri(trifluoromethyl)phenyl, 2,3,6-tri(trifluoromethyl)-phenyl, 2,4,6-tri(trifluoromethyl)phenyl, 2,3,4,5-tetra(trifluoro-methyl)phenyl, 2,3,4,6-tetra(trifluoro-methyl)phenyl, 2,3,4,5,6-penta(trifluoro-methyl)phenyl, 2-nitrophenyl, 3-nitrophenyl, 4-nitrophenyl, 2,3-dinitrophenyl, 2,4-dinitro-phenyl, 2,5-dinitrophenyl, 2,6-dinitrophenyl, 3,4-dinitrophenyl, 3,5-dinitro-phenyl, 2,3,4-trinitrophenyl, 2,3,5-trinitrophenyl, 2,3,6-trinitrophenyl, 2,4,6-trinitrophenyl, 2,3,4,5-tetranitrophenyl, 2,3,4,6-tetranitrophenyl, and 2,3,4,5,6-pentanitrophenyl.

29. The method of claim 13, wherein B is 2,4,5-trimethoxyphenyl.

30. The method of claim 13, wherein B is a substituted or unsubstituted heterocyclic ring chosen from:

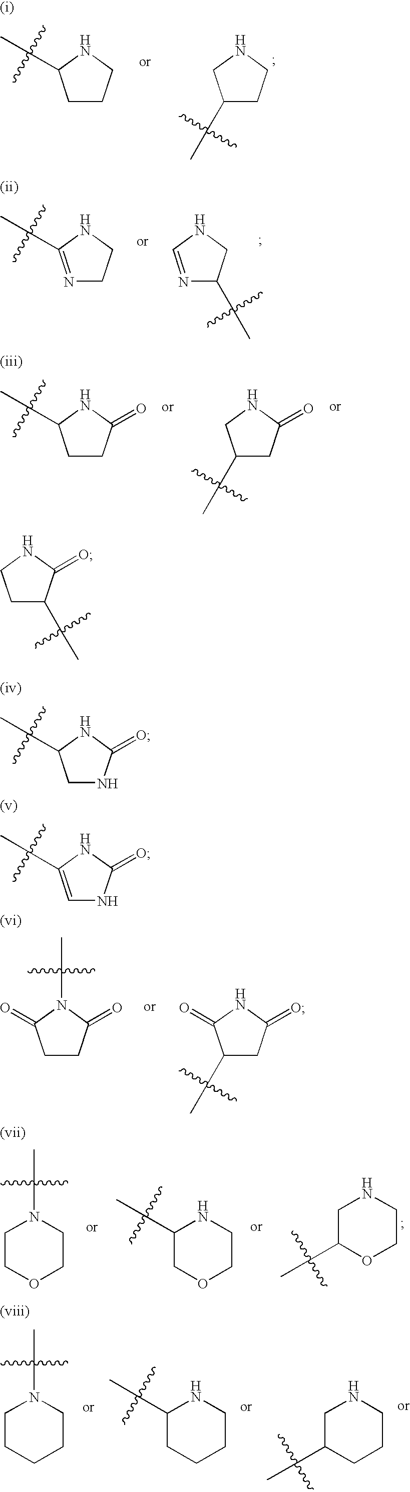

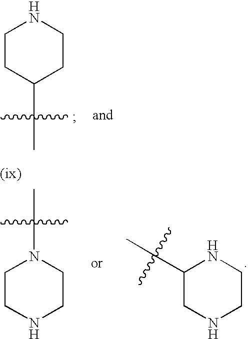

31. The method according to claim 13, wherein B is a substituted or unsubstituted cyclohexyl ring.

32. The method according to claim 31, wherein B is a cyclohexyl ring.

33. The method according to claim 13, wherein L is an alkylene units having the formula:
—[C(R^{6a}R^{6b})]_{w}—
wherein R^{6a} and R^{6b} are each independently chosen from hydrogen or methyl, and the index w is from 1 to 6.

34. The method according to claim 33, wherein L is chosen from:
(i) —CH₂CH₂—;
(ii) —CH₂CH₂CH₂—;
(iii) —CH₂CH₂CH₂CH₂—;
(iv) —CH₂CH(CH₃)CH₂—;
(v) —CH₂CH(CH₃)CH₂CH₂—;
(vi) —CH₂CH₂CH(CH₃)CH₂—; and
(vii) —CH₂CH₂CH₂CH₂CH₂CH₂—.

35. The method according to claim 13, wherein L comprises from 1 to 5 carbon atoms and one or more heteroatoms chosen from nitrogen, oxygen, or sulfur.

36. The method according to claim 35, wherein L is chosen from:
(i) —NHCH₂CH₂—;
(ii) —NHC(O)CH₂CH₂—;
(iii) —CH₂C(O)NHCH₂—;
(iv) —CH(CH₃)C(O)NHCH₂—;
(v) —CH₂C(O)NHCH(CH₃)—;
(vi) —CH(CH₃)C(O)NHCH(CH₃)—;
(vii) —CH₂OCH₂CH₂—; and
(viii) —CH₂SCH₂CH₂—.

37. The method according to claim 13, wherein L¹ is an alkylene units having the formula:
—[C(R^{15a}R^{15b})]_{z}—
wherein R^{15a} and R^{15b} are each independently chosen from hydrogen or methyl, and the index z is from 1 to 6.

38. The method according to claim 37, wherein L¹ is chosen from:
(i) —CH₂CH₂—;
(ii) —CH₂CH₂CH₂—;
(iii) —CH₂CH₂CH₂CH₂—;
(iv) —CH₂CH(CH₃)CH₂—;
(v) —CH₂CH(CH₃)CH₂CH₂—;
(vi) —CH₂CH₂CH(CH₃)CH₂—; and
(vii) —CH₂CH₂CH₂CH₂CH₂CH₂—.

39. The method according to claim 13, wherein L¹ comprises from 1 to 5 carbon atoms and one or more heteroatoms chosen from nitrogen, oxygen, or sulfur.

40. The method according to claim 39, wherein L¹ is chosen from:
(i) —CH₂S—;
(ii) —CH(CH₃)S—;
(iii) —CH₂SCH₂CH₂—;
(iv) —CH(CH₃)SCH₂CH₂—;
(v) —CH₂O—;
(vi) —CH(CH₃)O—;
(vii) —CH₂OCH₂CH₂—;
(viii) —CH(CH₃)OCH₂CH₂—; and
(ix)-CH₂CH₂OCH₂CH₂O—.

41. The method according to claim 13, wherein the activator is chosen from:
2,4,5-trimethoxy-N-(1H-1,2,4-triazol-3-yl)benzamide;
2-(2,5-dioxopyrrolidin-1-yl)-N-[4-(pyridine-2-yl)thiazol-2-yl]acetamide;
3-cyclohexyl-N-(1H-1,2,4-triazol-3-yl)propanamido;
2-(phenylthio)-N-(1H-1,2,4-triazol-3-yl)acetamide; and
3-phenyl-N-(1H-1,2,4-triazol-3-yl)propanamido.

42. The method of claim 1, wherein the TNAP activator is a compound having the formula: 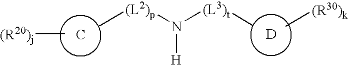
wherein C is a 5-member heterocyclic or heteroaryl ring that can optionally have from 1 to 4 hydrogen atoms substituted by an organic radical, R²⁰;
D represents a phenyl, cyclopentyl, cyclohexyl, or a 5-member heterocyclic ring can optionally have from 1 to 5 hydrogen atoms substituted by an organic radical, R³⁰;
L and L³ are each independently a linking unit having in the chain from 1 to 6 carbon atoms or from 1 to 5 carbon atoms together with from 1 to 4 heteroatoms chosen from nitrogen, oxygen, or sulfur;
the index k is from 1 to 5;
the index j from 1 to 4;
the index p is 0 or 1; and
the index t is 0 or 1.

43. The method according to claim 42, having the formula:

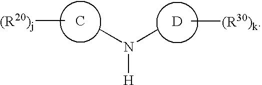

44. The method according to either claim 42, wherein C is a substituted or unsubstituted heteroaryl ring chosen from:

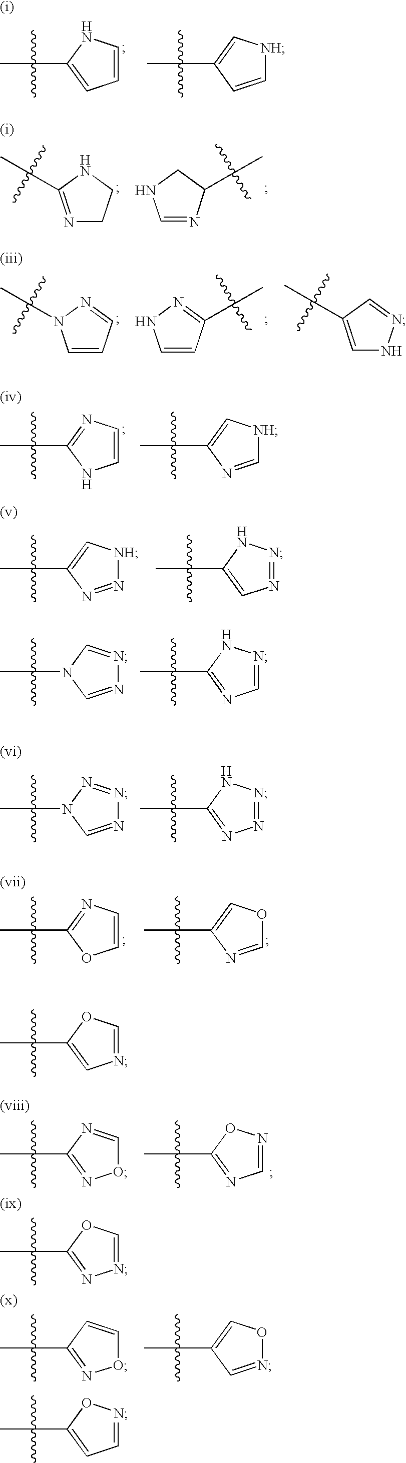

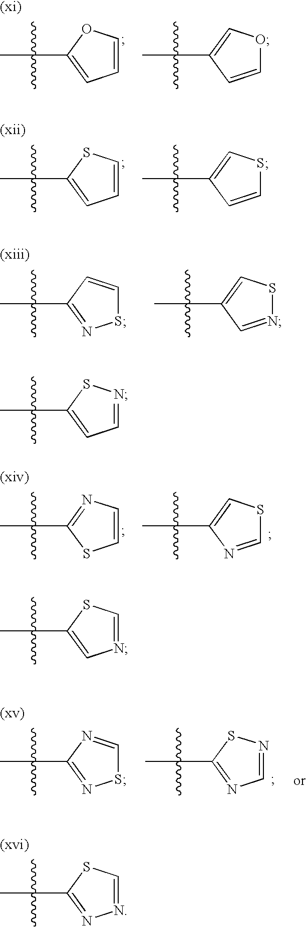

45. The method according to claim 44, wherein the heteroaryl ring can be substituted by from 1 to 4 R²⁰ organic radicals chosen from:
(i) linear, branched, or cyclic alkyl, alkenyl, and alkynyl;
(ii) substituted or unsubstituted aryl;
(iii) substituted or unsubstituted heterocyclic;
(iv) substituted or unsubstituted heteroaryl;
(v) —(CR^{23a}R^{23b})_{q}OR²²;
(vi) (CR^{23a}R^{23b})_{q}C(O)R²²;
(vii) —(CR^{23a}R^{23b})ₙC(O)OR²²;
(viii) —(CR^{23a}R^{23b})_{q}C(O)N(R²²);
(ix) (CR^{23a}R^{23b})_{q}OC(O)N(R²²)₂;
(x) —(CR^{23a}R^{3b})_{q}N(R²²)₂;
(xi) halogen;
(xii) —CHₘXₙ; wherein X is halogen, m is from 0 to 2, m+n=3;
(xiii) —(CR^{23a}R^{23b})_{q}CN;
(xiv) —(CR^{23a}R^{23b})_{q}NO₂;
(xv) —(CR^{23a}R^{23b})_{q}SO₂R²²; and
(xvi) —(CR^{23a}R^{23b})_{q}SO₃R²²;
wherein each R²² is independently hydrogen, substituted or unsubstituted C₁-C₄ linear, branched, or cyclic alkyl; or two R units can be taken together to form a ring comprising 3-7 atoms; R^{23a} and R^{23b} are each independently hydrogen or C₁-C₄ linear or branched alkyl; the index q is from 0 to 4.

46. The method of claim 45, wherein R²⁰ can be further substituted by one or more units chosen form:
(i) linear, branched, or cyclic alkyl, alkenyl, and alkynyl;
(ii) —(CR^{25a}R^{25b})_{q}OR²⁴;
(iii) —(CR^{25a}R^{25b})_{q}C(O)R²⁴;
(iv) —(CR^{25a}R^{25b})_{q}C(O)OR²⁴;
(v) —(CR^{25a}R^{25b})_{q}C(O)N(R²⁴⁾₂;
(vi) —(CR^{25a}R^{25b})_{q}OC(O)N(R²⁴)₂;
(vii) —(CR^{25a}R^{25b})_{q}N(R²⁴)₂;
(viii) halogen;
(ix) —CHₘXₙ; wherein X is halogen, m is from 0 to 2, m+n=3;
(x) —(CR^{25a}R^{25b})_{q}CN;
(xi) —(CR^{25a}R^{25b})_{q}NO₂;
(xii) —(CR^{25a}R^{25b})_{q}SO₂R²⁴; and
(xiii) —(CR^{25a}R^{25b})_{q}SO₃R²⁴;
wherein each R²⁴ is independently hydrogen, substituted or unsubstituted C₁-C₄ linear, branched, or cyclic alkyl; or two R²⁴ units can be taken together to form a ring comprising 3-7 atoms; R^{25a} and R^{25b} are each independently hydrogen or C₁-C₄ linear or branched alkyl; the index p is from 0 to 4.

47. The method of claim 42, wherein D is a substituted or unsubstituted 6 member heteroaryl ring chosen from:

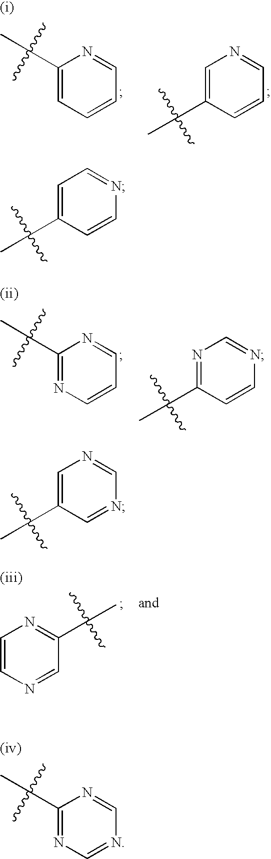

48. The method according to claim 42, wherein the compound has the formula: 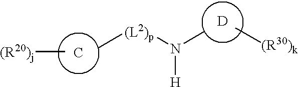
wherein C is substituted or unsubstituted phenyl or a substituted or unsubstituted heteroaryl ring having from 6 to 10 atoms and D is a substituted or unsubstituted heteroaryl ring having from 6 to 10 atoms.

49. The method according to claim 48, wherein the heteroaryl ring is chosen from:

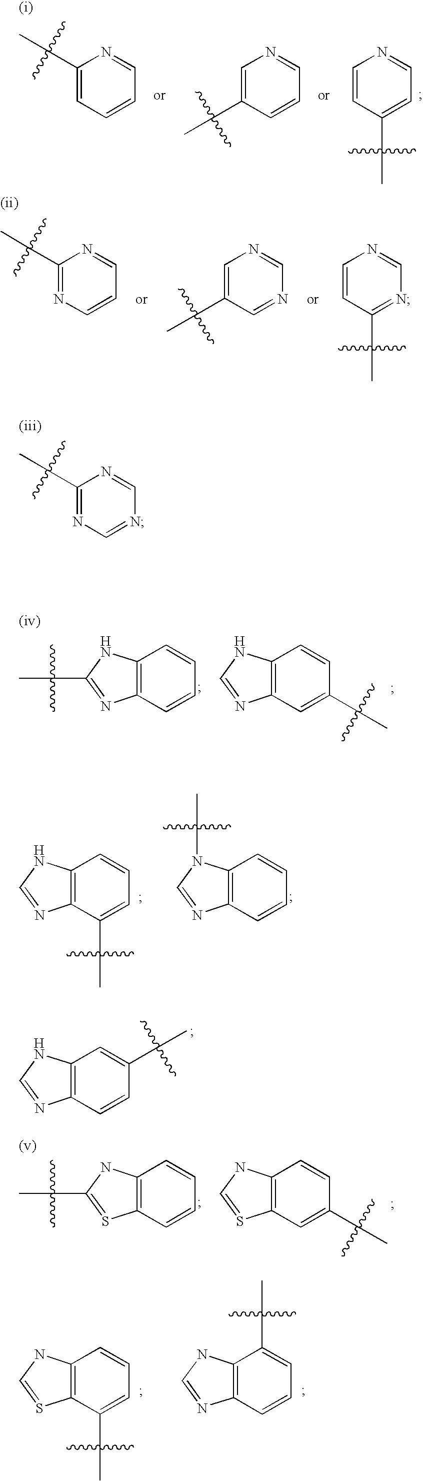

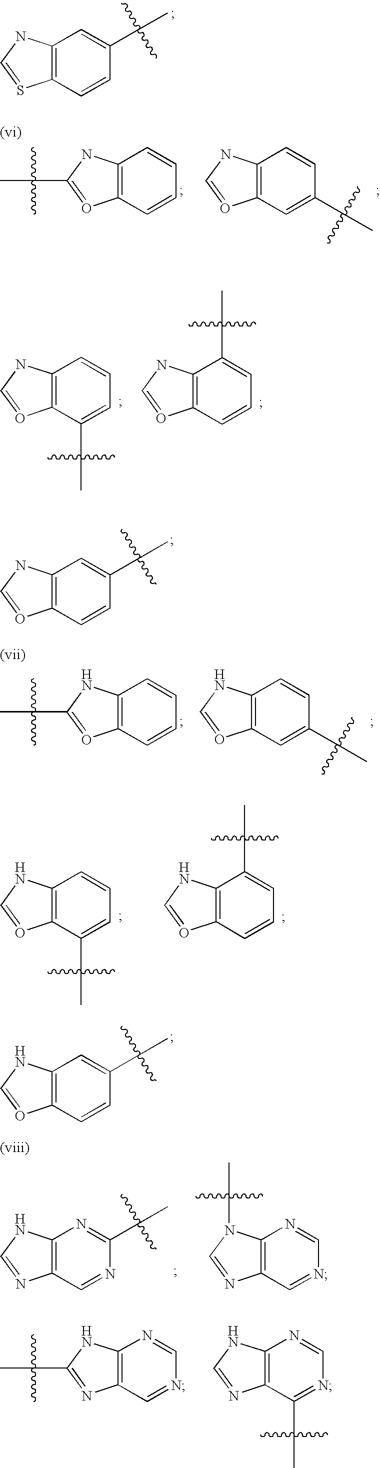

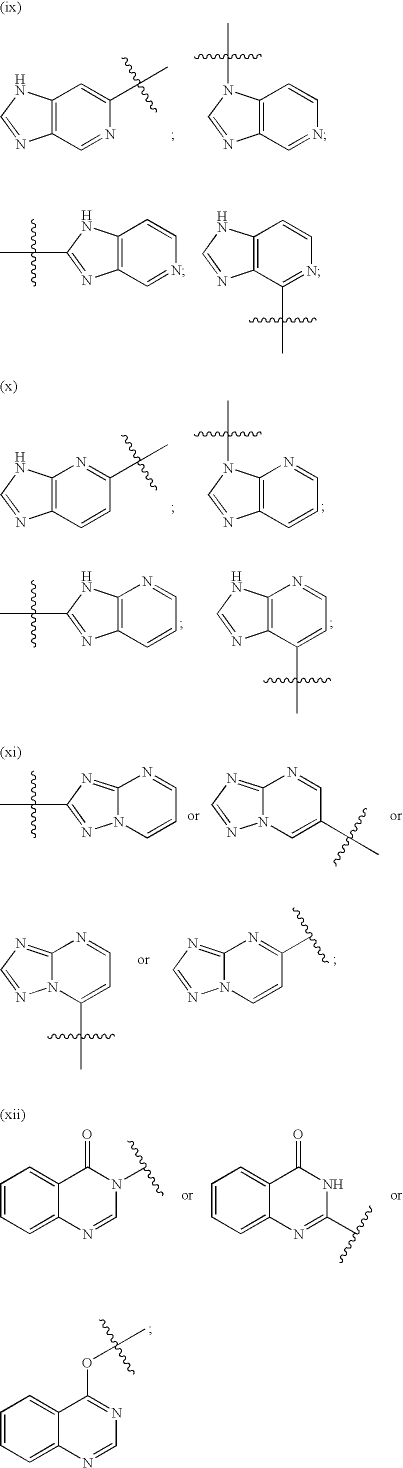

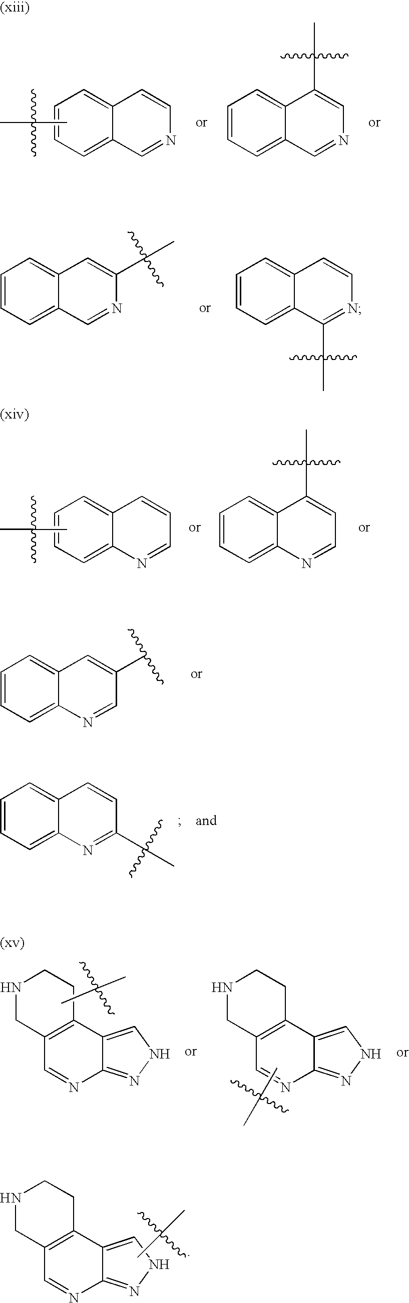

50. The method according to claim 42, wherein R³⁰ is an organic radical chosen from:
(i) linear, branched, or cyclic alkyl, alkenyl, and alkynyl;
(ii) substituted or unsubstituted aryl;
(iii) substituted or unsubstituted heterocyclic;
(iv) substituted or unsubstituted heteroaryl;
(v) —(CR^{33a}R^{33b})_{q}OR³²;
(vii) —(CR^{33a}R^{33b})_{q}C(O)R³²;
(viii) —(CR^{33a}R^{33b})_{q}C(O)OR³²;
(ix) —(CR^{33a}R^{33b})_{q}OC(O)N(R³²)₂;
(x) —(CR^{33a}R^{33b})_{q}N(R³²);
(xi) halogen;
(xii) —CHₘXₙ; wherein X is halogen, m is from 0 to 2, m+n=3;
(xiii) —(CR^{33a}R^{33b})_{q}CN;
(xiv) —(CR^{33a}R^{33b})_{q}NO₂;
(xv) —(CR^{33a}R^{33b}) SO₂R³²; and
(xvi) —(CR^{33a}R^{33b})_{q}SO₃R³²;
wherein each R³² is independently hydrogen, substituted or unsubstituted C₁-C₄ linear, branched, or cyclic alkyl; or two R³² units can be taken together to form a ring comprising 3-7 atoms; R^{33a} and R^{33b} are each independently hydrogen or C₁-C₄ linear or branched alkyl; the index q is from 0 to 4.

51. The method of claim 48, wherein R³⁰ can be substituted by one or more organic radicals independently chosen from:
(i) linear, branched, or cyclic alkyl, alkenyl, and alkynyl;
(ii) —(CR^{35a}R^{35b})_{q}OR³⁴;
(iii) —(CR^{35a}R^{35b})_{q}C(O)R³⁴;
(iv) —(CR^{35a}R^{35b})_{q}C(O)OR³⁴;
(v) —(CR^{35a}R^{35b})_{q}C(O)N(R³⁴)₂;
(vi) —(CR^{35a}R^{35b})_{q}OC(O)N(R³⁴)₂;
(vii) —(CR^{35a}R^{35b})_{q}N(R³⁴)₂;
(viii) halogen;
(ix) —CHₘXₙ; wherein X is halogen, m is from 0 to 2, m+n=3;
(x) —(CR^{35a}R^{35b})ₙCN;
(xi) —(CR^{35a}R^{35b})_{q}NO₂;
(xii) —(CR^{35a}R^{35b})_{q}SO₂R³⁴; and
(xiii) —(CR^{35a}R^{35b})_{q}SO₃R³⁴;
wherein each R³⁴ is independently hydrogen, substituted or unsubstituted C₁-C₄ linear, branched, or cyclic alkyl; or two R³⁴ units can be taken together to form a ring comprising 3-7 atoms; R^{35a} and R^{35b} are each independently hydrogen or C₁-C₄ linear or branched alkyl; the index p is from 0 to 4.

52. The method according to claim 42, wherein L² has the formula:
—[C(R2^{6a}R2^{6b})]ₛ-
wherein R^{26a} and R^{26b} are each independently chosen from hydrogen or methyl, and the index s is from 1 to 6.

53. The method according to claim 52, wherein L² is chosen from:
(i) —CH₂CH₂—;
(ii) —CH₂CH₂CH₂—;
(iii) —CH₂CH₂CH₂CH₂—;
(iv) —CH₂CH(CH₃)CH₂—;
(v) —CH₂CH(CH₃)CH₂CH₂—;
(vi) —CH₂CH₂CH(CH₃)CH₂—; and
(vii) —CH₂CH₂CH₂CH₂CH₂CH₂—.

54. The method according to claim 42, wherein L² comprises from 1 to 5 carbon atoms and one or more heteroatoms chosen from nitrogen, oxygen, or sulfur.

55. The method according to claim 54, wherein L² is chosen from:
(i) —NHCH₂CH₂—;
(ii) —NHC(O)CH₂CH₂—;
(iii) —CH₂C(O)NHCH₂—;
(iv) —CH(CH₃)C(O)NHCH₂—;
(v) —CH₂C(O)NHCH(CH₃)—;
(vi) —CH(CH₃)C(O)NHCH(CH₃)—;
(vii) —CH₂OCH₂CH₂—; and
(viii) —CH₂SCH₂CH₂—.

56. The method according to claim 42, wherein L³ has the formula:
—[C(R^{35a}R^{35b})]ᵣ—
wherein R^{35a} and R^{35b} are each independently chosen from hydrogen or methyl, and the index r is from 1 to 6.

57. The method according to claim 56, wherein L³ is chosen from:
(i) —CH₂CH₂—;
(ii) —CH₂CH₂CH₂—;
(iii) —CH₂CH₂CH₂CH₂—;
(iv) —CH₂CH(CH₃)CH₂—;
(v) —CH₂CH(CH₃)CH₂CH₂—;
(vi) —CH₂CH₂CH(CH₃)CH₂—; and
(vii) —CH₂CH₂CH₂CH₂CH₂CH₂—.

58. The method according to claim 42, wherein L³ comprises from 1 to 5 carbon atoms and one or more heteroatoms chosen from nitrogen, oxygen, or sulfur.

59. The method according to claim 58, wherein L³ is chosen from:
(i) —CH₂S—;
(ii) —CH(CH₃)S—;
(ii) —CH₂SCH₂CH₂—;
(iv) —CH(CH₃)SCH₂CH₂—;
(v) —CH₂O—;
(vi) —CH(CH₃)O—;
(vii) —CH₂OCH₂CH₂—;
(viii) —CH(CH₃)OCH₂CH₂—; and
(ix)-CH₂CH₂OCH₂CH₂O—.

60. The method according to claim 42, wherein the activator is chosen from:
N-(6-methylpyridin-2-yl)-4-(pyridine-2-yl)thiazol-2-amine;
1-isopropyl-N-[(1-methyl-1H-benzo[d]imidazol-2-yl)methyl]-1H-benzo[d]imidazol-2-amine;
5-(4-methoxyphenyl)-N-(pyridine-2-ylmethyl)-[1,2,4]triazole[1,5-a]pyrimidin-7-amine; and
N⁵,7-dibenzyl-6,7,8,9-tetrahydro-2H-pyrazolo[3,4-c][2,7]naphthyridine-1,5-diamine.

61. The method according to claim 1, wherein the TNAP activator is a substituted heteroaryl rings comprising from 5 to 11 atoms, wherein the heteroatom can be one or more nitrogen, oxygen, or sulfur atoms.

62. The method according to claim 61, wherein the heteroaryl rings can be substituted by one or more organic radicals independently chosen from:
(i) linear, branched, or cyclic alkyl, alkenyl, and alkynyl;
(ii) substituted or unsubstituted aryl attached to the heteroaryl ring by a polyalkylene tether having from 1 to 6 carbon atoms in the chain;
(iii) substituted or unsubstituted heterocyclic attached to the heteroaryl ring by a polyalkylene tether having from 1 to 6 carbon atoms in the chain;
(iv) substituted or unsubstituted heteroaryl attached to the heteroaryl ring by a polyalkylene tether having from 1 to 6 carbon atoms in the chain;
(v) —(CR^{43a}R^{43b})_{q}OR⁴²;
(vi) —(CR^{43a}R^{43b})_{q}C(O)OR⁴²;
(vii) —(CR^{43a}R^{43b})_{q}C(O)R⁴²;
(viii) —(CR^{43a}R^{43b})_{q}C(O)N(R⁴²)₂;
(ix) —(CR^{43a}R^{43b})_{q}OC(O)N(R⁴²)₂;
(X) —(CR^{43a}R^{43b})_{q}N(R⁴²)₂;
(xi) halogen;
(xii) —CHₘXₙ; wherein X is halogen, m is from 0 to 2, m+n=3;
(xiii) —(CR^{43a}R^{43b})_{q}CN;
(xiv) —(CR^{43a}R^{43b})_{q}NO₂;
(xv) —(CR^{43a}R^{43b})_{q}SO₂R⁴²; and
(v) —(CR^{43a}R^{43b})_{q}SO₃R⁴²;
wherein each R⁴² is independently hydrogen, substituted or unsubstituted C₁-C₄ linear, branched, or cyclic alkyl; or two R⁴² units can be taken together to form a ring comprising 3-7 atoms; R^{43a} and R^{43b} are each independently hydrogen or C₁-C₄ linear or branched alkyl; the index q is from 0 to 4.

63. The method according to claim 61, wherein the organic radicals that substitute for hydrogen on the heteroaryl ring can be further substituted by one or more organic radicals chosen from:
(i) linear, branched, or cyclic alkyl, alkenyl, and alkynyl;
(ii) —(CR^{45a}R^{45b})_{q}OR^{4r};
(iii) —(CR^{45a}R^{45b})_{q}C(O)R^{4r};
(iv) —(CR^{45a}R^{45b})_{q}C(O)OR^{4r};
(v) —(CR^{45a}R^{45b})_{q}C(O)N(R^{4r})₂;
(vi) —(CR^{45a}R^{45b})_{q}OC(O)N(R^{4r})₂;
(vii) —(CR^{45a}R^{45b})_{q}N(R^{4r})₂;
(viii) halogen;
(ix) —CHₘXₙ; wherein X is halogen, m is from 0 to 2, m+n=3;
(x) —(CR^{45a}R^{45b})_{q}CN;
(xi) —(CR^{45a}R^{45b})_{q}NO₂;
(xii) —(CR^{45a}R^{45b})_{q}SO₂R^{4r}; and
(xiii) —(CR^{45a}R^{45b})_{q}SO₃R^{4r};
wherein each R^{4r} is independently hydrogen, substituted or unsubstituted C₁-C₄ linear, branched, or cyclic alkyl; or two R^{4r} units can be taken together to form a ring comprising 3-7 atoms; R^{45a} and R^{45b} are each independently hydrogen or C₁-C₄ linear or branched alkyl; the index p is from 0 to 4.

64. The method according to claim 61, wherein the TNAP activator is chosen from:
(i) 3-[3-(1H-imidazol-1-yl)propyl]-7-benzyl-5,6-diphenyl-3H-pyrrolo[2,3-d]pyrimidin-4(7H)-imine: 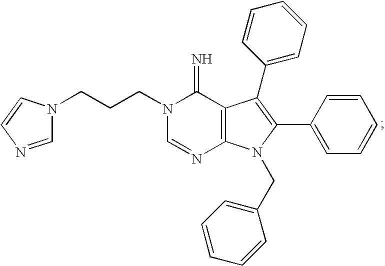
(ii) 7-(diethylamino)-3-(1-methyl-1H-benzo[d]imidazol-2-yl)-2H-chromen-2-one: 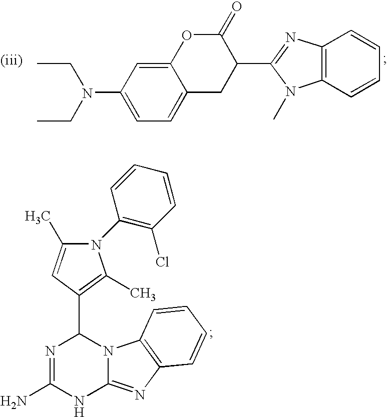
(iv) 5-tert-butyl-2-methyl-3-phenylpyrazolo[1,5-a]pyrimidin-7-ol: 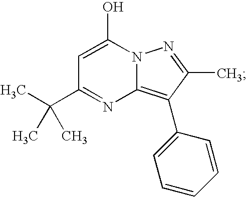
(v) 7-[morpholino(pyridine-2-yl)methyl]quinolin-8-ol: 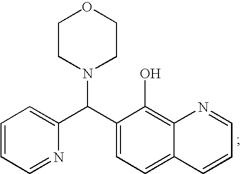
(vi) 2,2′,2″,2′″-[4,8-di(piperidin-1-yl)pyrimido[5,4-d]pyrimidine-2,6-diyl]bis(azanetriyl)tetraethanol; 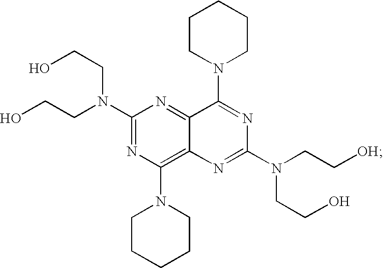
(vii) 3-(3-phenylpyridazino[3,4-b]quinoxalin-5(10H)-yl)propan-1-ol: 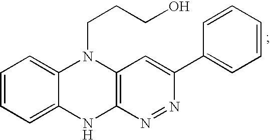
(viii) 6-cyclohexyl-3-(2,4,5,6-tetrahydrocyclopenta[c]pyrazol-3-yl)-[1,2,4]triazole[3,4-b][1,3,4]thiadiazole: 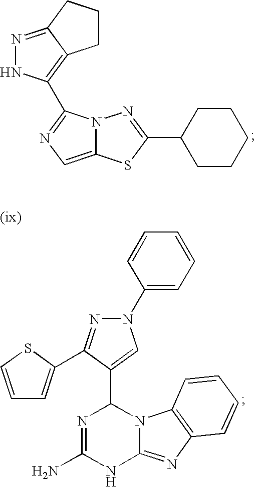
(x) 5,5,7,12,12,14-hexamethyl-1,4,8,11-tertraazacyclotetradecane: 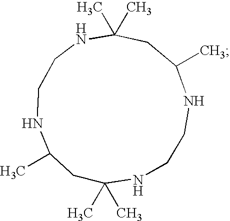
(xi) 2,2′,2″-(1-oxa-4,7,10-triazacyclododecane-4,7,10-triyl)ethanol 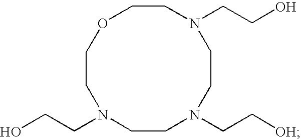
(xii) N-(3,4-dimethoxyphenethyl)-5-(2-hydroxyphenyl)-1H-pyrazole-3-carboxamide 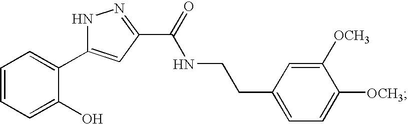
(xiii) N-[2-(4-fluorobenzylamino)-2-oxoethyl]-2-(4-fluorphenylsulfonamido)-N-(furan-2-ylmethyl)acetamide 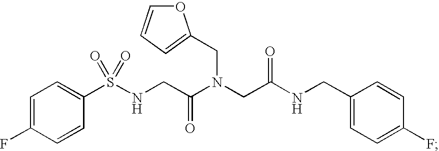
(xiv) 5-bromo-N-[3-(trifluoromethoxy)phenyl]furan-2-carboxamide 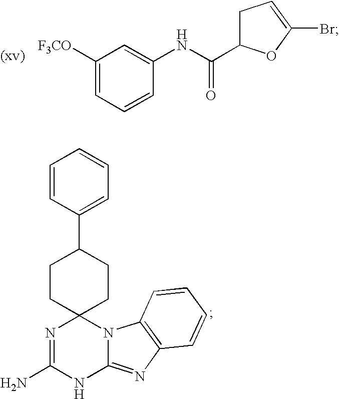
(xvi) 2-[2-(naphthalene-2-ylsulfonyl)ethyl]-5-phenyl-1,3,4-oxadiazole 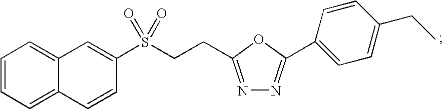
(xvii) N-{2-[ethyl(phenyl)amino]ethyl}-1-{[2-(4-ethylphenyl)-5-methyloxazol-4-yl]methyl}piperidine-4-carboxamide 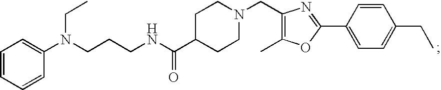
or
(xviii) N-[1-(2,6-dimethylphenylcarbamoyl)cyclohexyl]-N-(3-methoxyphenyl)-1H-pyrazole-3-carboxamide

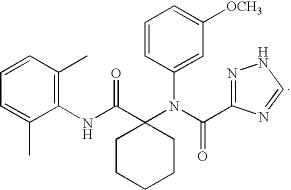

65. The method of claim 1, wherein the subject is suffering hypophosphatasia.

66. The method of claim 1, wherein the subject is suffering osteoporosis.

67. The method of claim 1, wherein the subject is suffering calcium pyrophosphate deposition disease (CPPD/chodrocalcinosis).
